(19) United States
(12) Patent Application Publication
(10) Pub. No.: US 2021/0077399 A1
(43) Pub. Date: Mar. 18, 2021

(54) MAGNETIC LIPOSOMES AND RELATED TREATMENT AND IMAGING METHODS

(71) Applicant: UNIVERSITY OF FLORIDA RESEARCH FOUNDATION, INCORPORATED, Gainesville, FL (US)

(72) Inventors: Adam J. Grippin, Gainesville, FL (US); Duane Mitchell, Gainesville, FL (US); Jon P. Dobson, Gainesville, FL (US); Elias Sayour, Gainesville, FL (US); Adam Monalve, Gainesville, FL (US)

(21) Appl. No.: 17/053,691

(22) PCT Filed: May 8, 2019
(86) PCT No.: PCT/US19/31385

§ 371 Date: Nov. 6, 2020

Related U.S. Application Data
(60) Provisional application No. 62/668,608, filed on May 8, 2018.

(51) Int. Cl. A61K 9/127; A61K 49/18; A61K 31/713

(52) U.S. Cl. CPC A61K 9/1272; A61K 31/713; A61K 9/1277; A61K 49/1812

(57) ABSTRACT

Provided herein is a liposome comprising ribonucleic acid (RNA) molecules, a lipid mixture comprising DOTAP and cholesterol, and iron oxide nanoparticles (IONPs). Also provided herein is a liposome comprising ribonucleic acid (RNA) molecules and a lipid mixture comprising DOTAP and cholesterol, wherein the DOTAP and cholesterol are present in the lipid mixture at a DOTAP:cholesterol ratio of about 3:1 by mass. Related cells comprising the liposome, populations of cells, and compositions are also provided. Methods of making a liposome and methods of using the liposome are further provided.

### CROSS-REFERENCE TO RELATED APPLICATIONS

This application claims priority to U.S. Provisional Patent Application No. 62/668,608, filed May 8, 2018, the contents of which are incorporated by reference in its entirety.

### GRANT FUNDING DISCLOSURE

This invention was made with government support under CA195563 awarded by National Institutes of Health. The government has certain rights in the invention.

### BACKGROUND OF THE INVENTION

Cancer vaccines are a promising approach to personalized cancer immunotherapy, but the lack of meaningful biomarkers of patient response to treatment limit their development. In a randomized, double blind, placebo-controlled trial, RNA-pulsed dendritic cells (DCs) were reported to prolong progression-free and overall survival in patients with glioblastoma (Mitchell et al, Nature 519: 366-369 (2015)). Furthermore, DC migration to lymph nodes assessed by SPECT/CT imaging was demonstrated to strongly correlate with clinical outcomes. While this finding may provide a novel imaging biomarker for response to DC vaccines, the complexity and regulatory requirements of nuclear medicine-based imaging of radiolabeled cells limits widespread utilization of this technique. Therefore, there is a need in the art for methods to track DC migration to lymph nodes without radioactivity and methods to enhance migration to improve vaccination response.

### SUMMARY

Presented herein for the first time are data which culminates in the development of bi-functional RNA-loaded nanoparticles (RNA-NPs) that enhance DC migration to lymph nodes and enable MRI-based tracking of DC migration in vivo. Accordingly, the present disclosure provides a liposome comprising ribonucleic acid (RNA) molecules and a lipid mixture comprising DOTAP and cholesterol. In various embodiments, the liposomes comprise iron oxide nanoparticles (IONPs). In various aspects, the DOTAP and cholesterol are present in the lipid mixture at a DOTAP:cholesterol ratio of about 3:1 by mass. The various advantages of the presently disclosed liposomes are further described in detail below.

The present disclosure also provides a cell comprising a liposome of the present disclosure, e.g., an immune cell (e.g., an antigen presenting cell, a dendritic cell) comprising a liposome of the present disclosure. In various aspects, the cell is transfected with the liposome. In various instances, the cells has taken up by endocytosis or pinocytosis the liposomes of the present disclosure. Cells comprising the liposome of the present disclosure are not limited to any particular mechanism by which the liposome is taken up by the cell. Also provided is a population of cells, wherein at least 50% of the population are cells comprising a liposome of the present disclosure, e.g., cells transfected with a liposome of the present disclosure.

The present disclosure further provides a composition comprising a liposome, a cell (e.g., a dendritic cell), or a population of cells, of the present disclosure, or any combination thereof, and a pharmaceutically acceptable carrier, excipient or diluent.

The present disclosure additionally provides a method of making a liposome. In exemplary embodiments, the method comprises (A) mixing DOTAP and cholesterol at a DOTAP:cholesterol ratio of about 3:1 by mass to form a lipid mixture, (B) drying the lipid mixture, (C) rehydrating the lipid mixture with a rehydration solution to form a rehydrated lipid mixture, (D) incubating the rehydrated lipid mixture at a temperature greater than about 40° C. and intermittently vortexing the rehydrated lipid mixture to form liposomes. A liposome made by the method of the present disclosure is also provided. A cell comprising (e.g., transfected with) the liposome made by the method of the present disclosure is further provided herein. Also provided is a population of cells, wherein at least 50% of the population are cells comprising (e.g., transfected with) the liposome made by the method of the present disclosure. The present disclosure provides a composition comprising the liposome made by the method of the present disclosure, a cell comprising the liposome, or a population of cells, as described herein, or any combination thereof, and a pharmaceutically acceptable carrier, excipient or diluent.

The present disclosure further provides a method of delivering RNA molecules to cells. In exemplary embodiments, the method comprises incubating the cells with the liposomes of the present disclosure. In various aspects, the cells are immune cells. In various instances, the immune cells are antigen-presenting cells, e.g., dendritic cells. In various aspects, the immune cells are located in a tumor microenvironment, e.g., the tumor environment of a brain tumor. The liposomes of the present disclosure in some aspects activates the immune cells. In various instances, the RNA molecules are antisense molecules, e.g., siRNA, which target a protein of an immune checkpoint pathway. For instance, the protein of the immune checkpoint pathway may be PDL1. Accordingly, in various aspects, the siRNA targeting PDL1 is delivered to immune cells of the microenvironment. Without being bound to any particular theory, the liposomes activate the immune cells of the microenvironment and also reduce expression of the protein of the immune checkpoint pathway to enhance an immune response against the tumor. In various aspects, the RNA molecules encode a protein, e.g., a tumor antigen. In various aspects, the RNA molecules are mRNA encoding tumor antigens.

Methods of treating a subject with a disease are provided by the present disclosure. In exemplary embodiments, the method comprises delivering RNA molecules to cells of the subject by a presently disclosed method of delivering RNA molecules to cells. In other exemplary embodiments, the method comprises administering a presently disclosed composition comprising a liposome, a cell, a population of cells, as described herein, or any combination thereof, in an amount effective to treat the disease in the subject.

The present disclosure further provides methods of enhancing in a subject an immune response against a tumor or cancer. In exemplary embodiments, the method comprises administering to the subject a liposome of the present disclosure in an amount effective to enhance the immune response in the subject. In exemplary aspects, the enhanced immune response is evident by increased activation of dendritic cells which is demonstrated by, e.g., enhanced expression of genes related to DC activation, enhanced expression of co-stimulatory molecules on the surface of DCs, increased production of anti-viral cytokines (e.g., IFNα), increased T cell stimulation (as shown by e.g., increased IFN-γ production by T-cells upon contact with the activated DCs), increased migration to lymph nodes, and enhanced inhibition of tumor growth. Accordingly, the present disclosure further provides methods of increasing activation of DCs or activating DCs. In exemplary embodiments, the method comprises administering to the subject a liposome of the present disclosure in an amount effective to increase activation of DCs or in an amount effective to activate DCs. The present disclosure also accordingly provides methods of increasing production of anti-viral cytokines (e.g., IFNα). In exemplary embodiments, the method comprises administering to the subject a liposome of the present disclosure in an amount effective to increase production of the anti-viral cytokine in the subject. Further provided are methods of increasing T cell stimulation in a subject. In exemplary embodiments, the method comprises administering to the subject a liposome of the present disclosure in an amount effective to increase T cell stimulation in the subject. In various aspects, the increase in T-cell stimulation is evident from an increase in T-cell production of IFN-γ. The present disclosure further provides methods of increasing T cell production of IFN-γ. In exemplary embodiments, the method comprises contacting T cells with a presently disclosed dendritic cell (DC), optionally, wherein the liposome comprise IONPs and the DC is transfected with the liposome in the presence of a magnetic field. A method of increasing dendritic cell (DC) migration to a lymph node in a subject is additionally provided herein. In exemplary embodiments, the method comprises administering to the subject a presently disclosed composition in an amount effective to increase DC migration to the lymph node. Methods of inhibiting tumor growth are furthermore provided herein. In exemplary embodiments, the method comprises administering to the subject a liposome of the present disclosure in an amount effective to inhibit tumor growth in the subject.

The present disclosure also provides a method of tracking dendritic cell (DC) migration to a lymph node in a subject. In exemplary embodiments, the method comprises (i) treating the subject in accordance with a presently disclosed method of treating, wherein the cells are DCs and the liposomes comprise IONPs, and (ii) performing magnetic resonance imaging (MRI) on one or more lymph nodes of the subject. In exemplary aspects, the method comprises determining the T2*-weighted MRI intensity of one or more lymph nodes, wherein lymph nodes exhibiting a reduction in T2*-weighted MRI intensity, relative to the T2*-weighted MRI intensity of a control, untreated lymph node, represent lymph nodes to which DCs migrated. In exemplary embodiments, the methods of tracking DC migration to a lymph node in a subject comprises incubating DCs obtained from a subject with the liposomes of the present disclosure and administering the DCs comprising the liposomes to the subject. The methods further comprise conducting MRI on one or more lymph nodes of the subject following administration of the DCs to the subject. In various aspects, the MRI is conducted about 2 days following administration. As further described herein, the methods in some aspects comprise measuring T2*-weighted MRI intensity of treated lymph nodes and comparing the T2*-weighted MRI intensity to the intensity before administration of the DCs to the subject. In various instances, a reduction in the T2*-weighted MRI intensity is associated with a positive outcome (e.g., a positive therapeutic response) of the DC administration. Accordingly, a method of determining a subject's therapeutic response to dendritic cell (DC) vaccination therapy in a subject is provided by the present disclosure. In exemplary embodiments, the method comprises (i) treating the subject in accordance with a presently disclosed method of treating, wherein the cells are DCs and the liposomes comprise IONPs, and (ii) tracking DC migration to a lymph node in accordance with a presently disclosed method of tracking DC migration, wherein, when T2*-weighted MRI intensity of treated lymph nodes is reduced, the DC vaccination therapy is determined to lead to a positive therapeutic response in the subject.

The present disclosure also provides methods of monitoring therapeutic response to dendritic cell (DC) vaccination therapy in a subject. In exemplary embodiments, the method comprises tracking DC migration to a lymph node in accordance with a presently disclosed method of tracking DC migration to a lymph node at a first time point and at a second time point, wherein, when T2*-weighted MRI intensity of treated lymph nodes is reduced at the second time point relative to the T2*-weighted MRI intensity of the treated lymph nodes at the first time point, the therapeutic response to DC vaccination therapy is effective.

The present disclosure provides a method of delivering RNA to cells in a microenvironment of a tumor, optionally, a brain tumor, comprising intravenously administering a presently disclosed composition, wherein the composition comprises the liposome.

### DETAILED DESCRIPTION

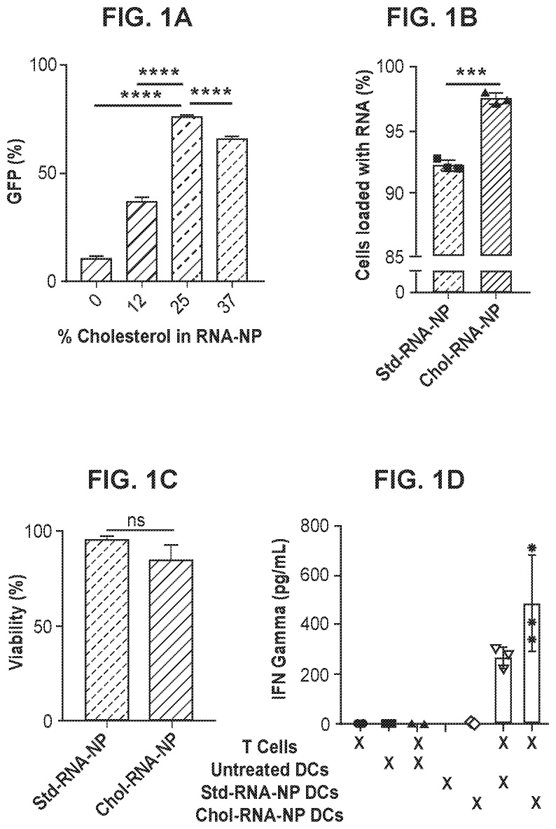

FIG. 1A is a graph of the percentage of DCs that are transfected with GFP RNA containing liposomes comprising different amounts of cholesterol.

FIG. 1B is a graph of the % of dendritic cells that were loaded with RNA delivered by liposomes comprising 0% cholesterol (Std-RNA-NP) or 25% cholesterol (Chol-RNA-NP).

FIG. 1C is a graph of the % of viable dendritic cells that were loaded with RNA delivered by liposomes comprising 0% cholesterol (Std-RNA-NP) or 25% cholesterol (Chol-RNA-NP).

FIG. 1D is a graph of the IFNγ produced by T-cells incubated with dendritic cells transfected with liposomes comprising 0% cholesterol and tumor-derived RNA (Std-RNA-NP DCs) or dendritic cells transfected with liposomes comprising 25% cholesterol and tumor-derived RNA liposomes (Chol-RNA-NP DCs). As scontrols, untransfected DCs (untreated DCs) and T cells were used in this experiment.

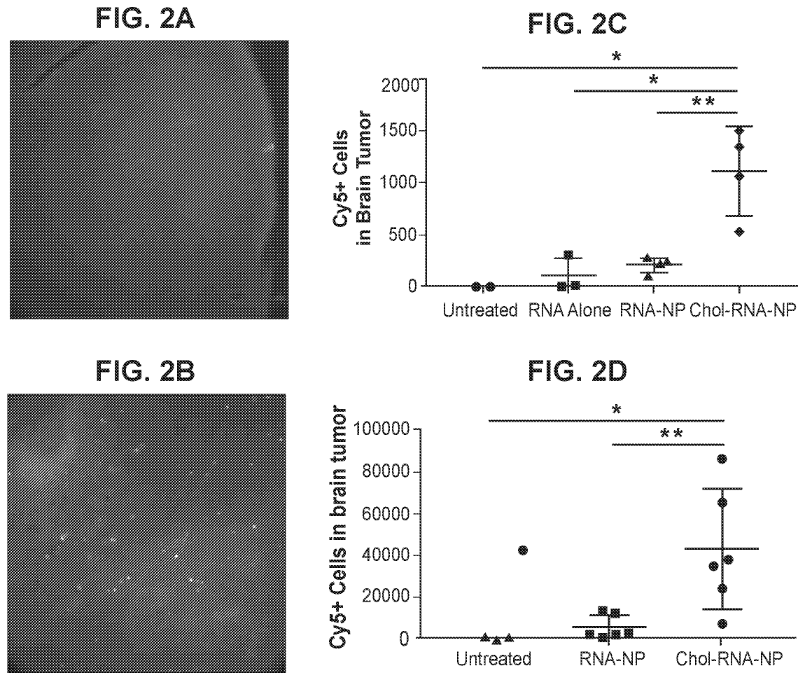

FIG. 2A is an immunofluorescence image of the cortex and FIG. 2B is an immunofluorescence image of the tumor.

FIG. 2C is a graph of the Cy5+ cells in the brain tumor of mice with KR158B-luciferase tumors and injected with liposomes comprising 25% cholesterol (Chol-RNA-NP), with 0% cholesterol (RNA-NP), RNA alone, or untreated.

FIG. 2D is a graph of the Cy5+ cells in the brain tumor of mice with GL261 tumors and injected with liposomes comprising 25% cholesterol (Chol-RNA-NP), with 0% cholesterol (RNA-NP), or untreated.

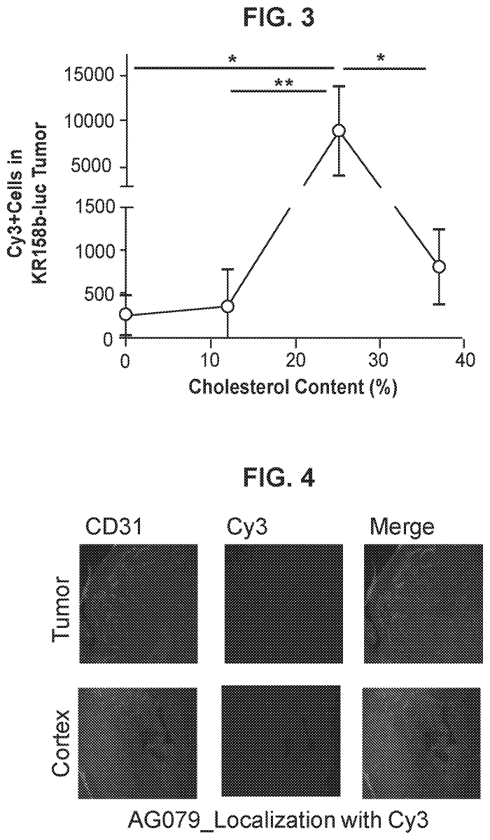

FIG. 3 is a graph of the Cy3+ cells in the tumors of mice with KR158B-luciferase tumors and injected with liposomes comprising 1%, 12.5%, 25% or 37% cholesterol, plotted as a function of cholesterol content.

FIG. 4 is a series of fluorescent microscope images of the tumor or cortex stained with CD31 fluorescent antibody (left column) or liposomes comprising Cy3-labeled RNA (middle column) or the merged images (right column).

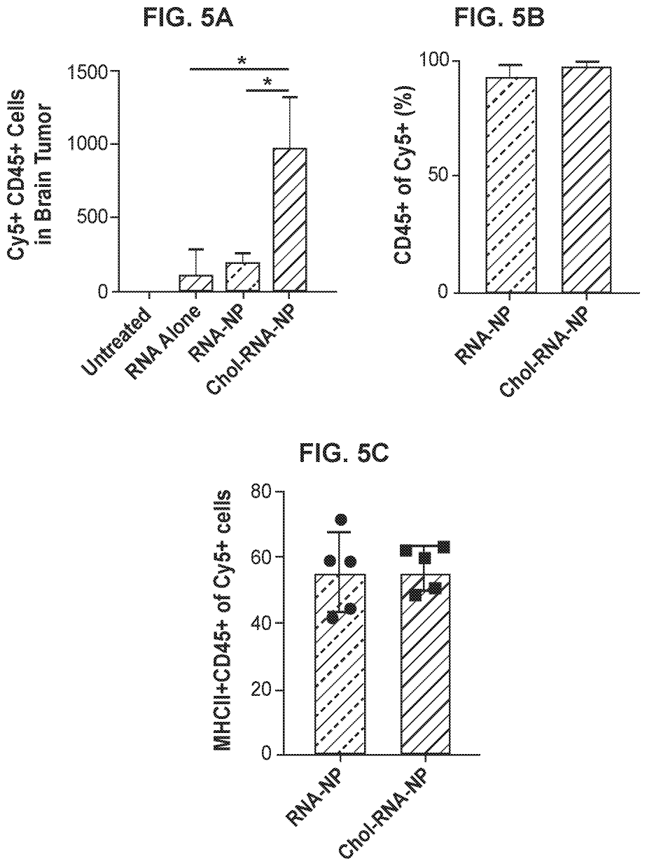

FIG. 5A is a graph of the Cy5+CD45+ cells in the brain tumor of mice injected with liposomes comprising 25% cholesterol (Chol-RNA-NP), with 0% cholesterol (RNA-NP), RNA alone, or untreated.

FIG. 5B is a graph of the % of CD45+ cells that are Cy5+ in the brain tumor of mice injected with liposomes comprising 25% cholesterol (Chol-RNA-NP) or with 0% cholesterol (RNA-NP).

FIG. 5C is a graph of the MHC Class II+ CD45+ cells that are Cy5+ in the brain tumor of mice injected with liposomes comprising 25% cholesterol (Chol-RNA-NP) or with 0% cholesterol (RNA-NP).

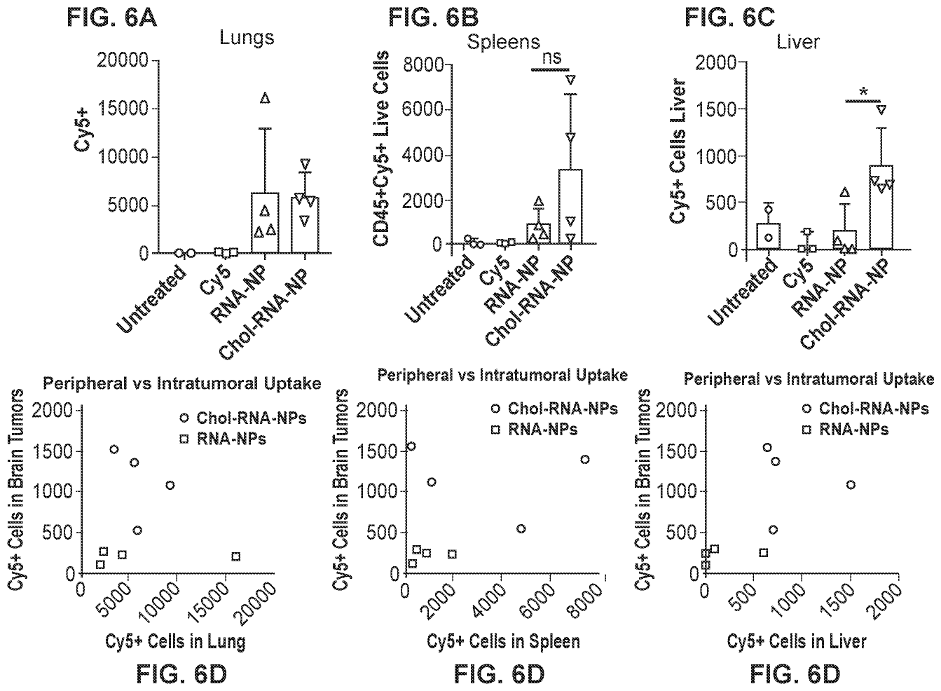

FIG. 6A is a graph of the Cy5+ cells in the lungs of mice injected with liposome comprising 25% cholesterol (Chol-RNA-NP), liposomes with 0% cholesterol (RNA-NP), untreated or RNA alone (Cy5).

FIG. 6B is a graph of the Cy5+ cells in the spleens of mice injected with liposome comprising 25% cholesterol (Chol-RNA-NP), liposomes with 0% cholesterol (RNA-NP), untreated or RNA alone (Cy5).

FIG. 6C is a graph of the Cy5+ cells in the liver of mice injected with liposome comprising 25% cholesterol (Chol-RNA-NP), liposomes with 0% cholesterol (RNA-NP), untreated or RNA alone (Cy5).

FIG. 6D is a graph of the Cy5+ cells in the brain tumors of mice injected with liposome comprising 25% cholesterol (Chol-RNA-NP-circles) or liposomes with 0% cholesterol (RNA-NP-squares), plotted as a function of Cy5+ cells in the lung.

FIG. 6E is a graph of the Cy5+ cells in the brain tumors of mice injected with liposome comprising 25% cholesterol (Chol-RNA-NP-circles) or liposomes with 0% cholesterol (RNA-NP-squares), plotted as a function of Cy5+ cells in the spleen.

FIG. 6F is a graph of the Cy5+ cells in the brain tumors of mice injected with liposome comprising 25% cholesterol (Chol-RNA-NP-circles) or liposomes with 0% cholesterol (RNA-NP-squares), plotted as a function of Cy5+ cells in the liver.

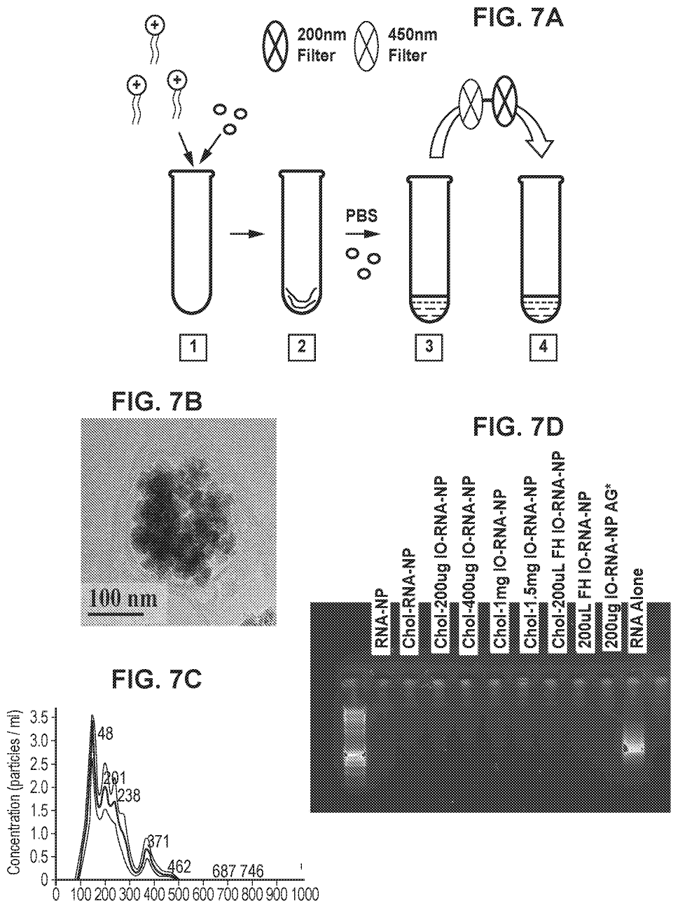

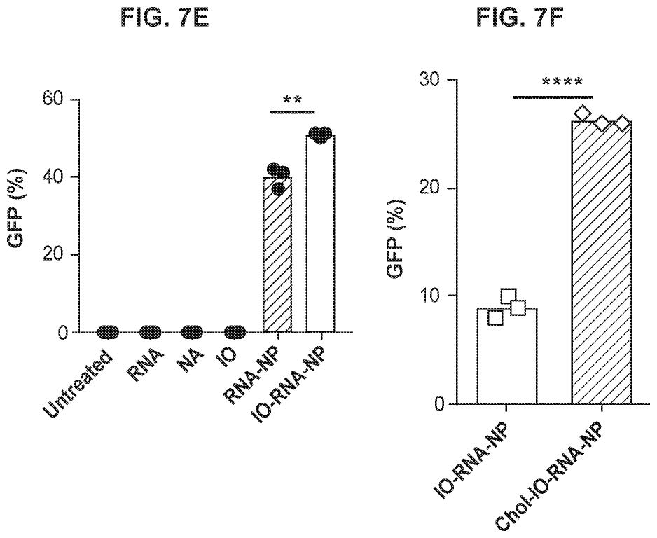

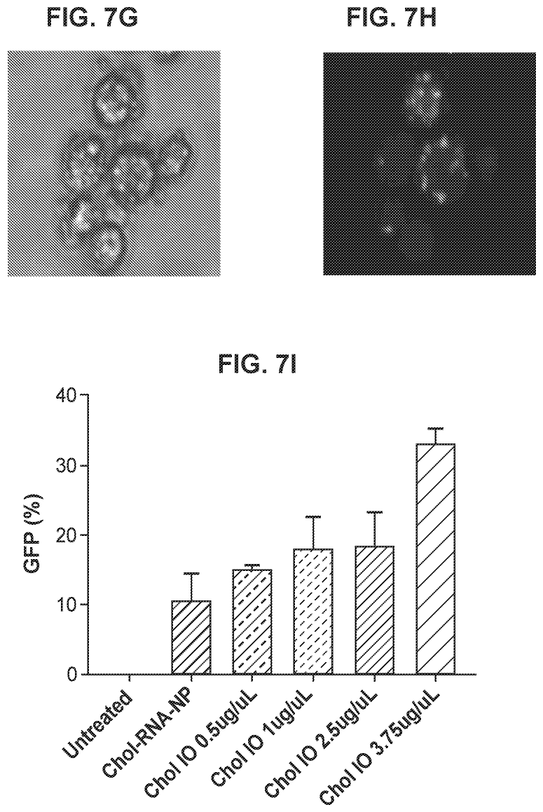

FIG. 7A is a schematic of the basic steps for making liposomes comprising iron oxide nanoparticles.

FIG. 7B is a Cryo-TEM image of iron oxide liposomes.

FIG. 7C is a graph of the concentration of particles plotted as a function of particle diameter.

FIG. 7D is an image of an agarose gel, the wells of which were loaded with IO-RNA-NPs containing various amounts of iron oxide. In this image, bright bands indicate the presence of unbound mRNA. The absence of a band in a column indicates that all RNA was bound by that particle formulation.

FIG. 7E is a graph of the % of cells transfected with liposomes comprising green fluorescent protein (GFP) RNA with iron oxide (IO-RNA-NP) or without iron oxide (RNA-NP) or with iron oxide particles alone or with nanoparticles alone or untreated.

FIG. 7F is a graph of the % of DCs transfected with liposomes comprising iron oxide and cholesterol (Chol-IO-RNA-NP) or with iron oxide but without cholesterol (IO-RNA-NP).

FIG. 7G is a bright field image of DCs transfected with Cy3-labelled IO-RNA-NPs.

FIG. 7H is a fluorescent image of DCs transfected with Cy3-labelled IO-RNA-NPs.

FIG. 7I is a graph of the percentage of DCs that are transfected with GFP RNA containing liposomes comprising cholesterol and differing amounts of carboxylated iron oxide nanoparticles.

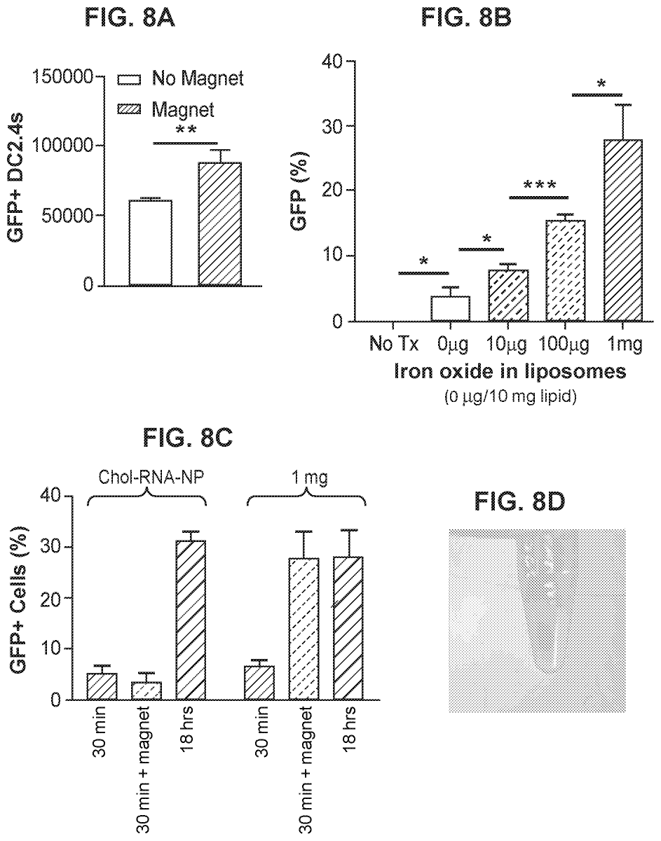

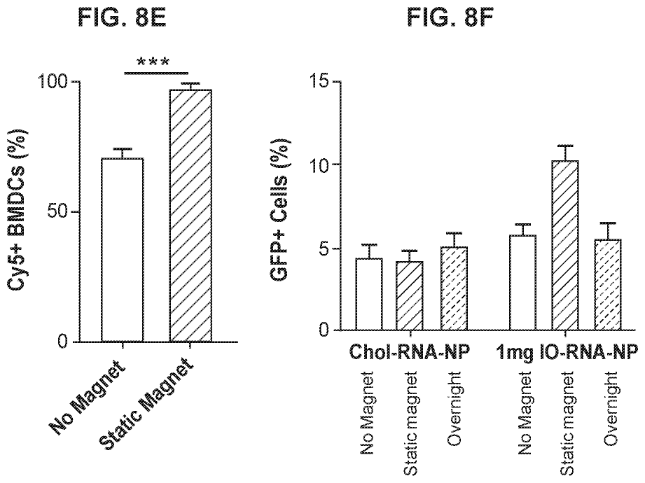

FIG. 8A is a graph of percentage of DCs that are transfected with GFP RNA containing iron oxide liposomes in the presence (magnet) or absence of a magnetic field (no magnet).

FIG. 8B is a graph of the percentage of DCs that are transfected with GFP RNA containing iron oxide liposomes having varying amounts of iron oxide in the presence of a magnetic field.

FIG. 8C is a graph of the percentage of DCs that are transfected with GFP RNA containing magnetic liposomes for 30 minutes in the presence (30 min+Magnet) or absence of a magnetic field (30 min) or overnight without a magnetic field (18 hours).

FIG. 8D is a photo of a tube comprising DCs placed on a magnetic separator for 30 min showing a visible mass of cells attracted to the side of the tube where the magnetic field is the greatest.

FIG. 8E is a graph of the % of Cy5+-positive bone marrow DCs incubated with magnetic liposomes in the presence (Static Magnet) or absence of a magnetic field (No Magnet).

FIG. 8F is a graph of the percentage of DCs that are transfected with GFP RNA containing JO liposomes in the presence (Static Magnet) or absence of a magnetic field (No Magnet) for 30 minutes or overnight (without a magnet; Overnight).

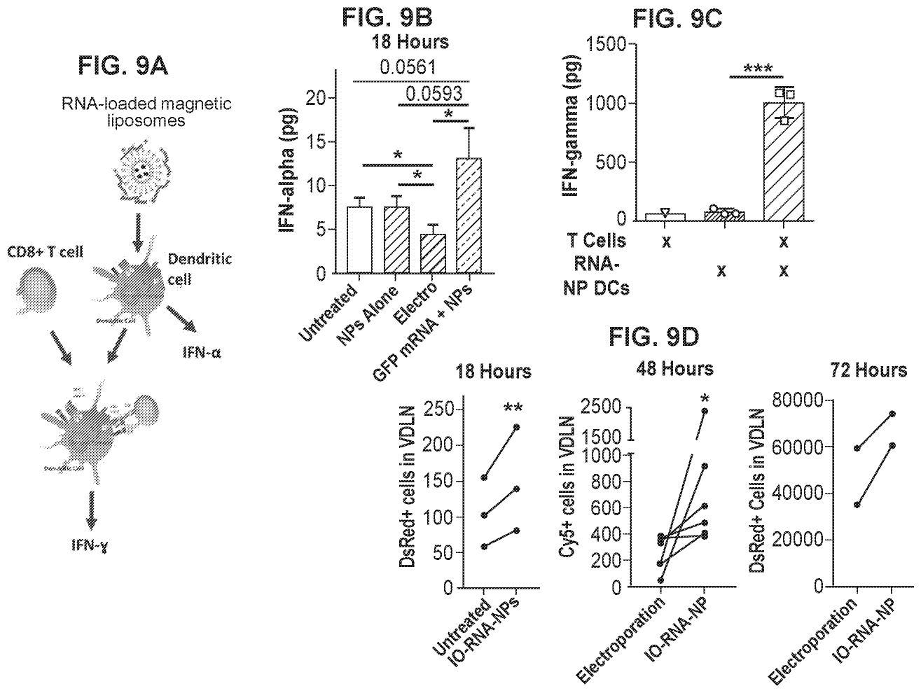

FIG. 9A is a diagram of pathways showing the effects of RNA-loaded magnetic liposomes on cytokine production.

FIG. 9B is a graph of the IFNα produced by DCs electroporated with GFP RNA (Electro), incubated with RNA- and IO-loaded liposomes (GFP mRNA+NPs), untreated or treated with just nanoparticles (NPs alone).

FIG. 9C is a graph of the IFNγ produced by T-cells stimulated with DCs incubated with RNA-loaded liposomes or unstimulated T cells.

FIG. 9D is a graph of the DsRed+ cells in lymph nodes of mice injected with DsRed DCs loaded with Cy3-labelled OVA RNA (18 hours) or Cy3-labeled GFP RNA via electroporation (left) or IO-RNA-NPs (right) after 18, 48 or 72 hours).

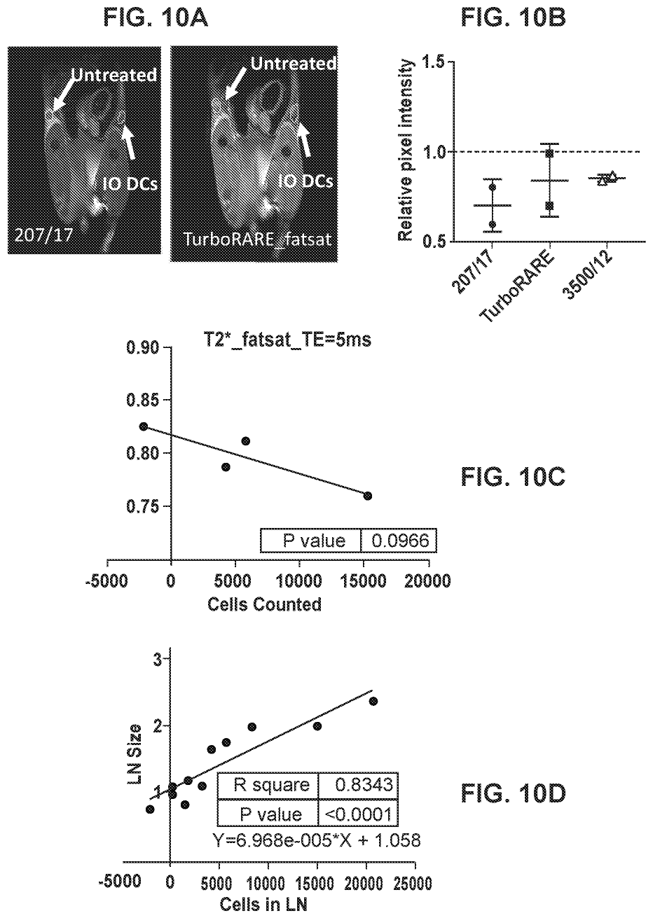

FIG. 10A are representative images of treated and untreated lymph nodes in T2* weighted images with TR/TE of 207/17 and T2_RARE weighted images for each set of imaging parameters.

FIG. 10B is a quantification of the data of FIG. 10A.

FIG. 10C is a graph of the number of cells in each lymph node plotted against relative change in intensity in T2*-weighted images with fat saturation.

FIG. 10D is a graph of the average relative change in lymph node size.

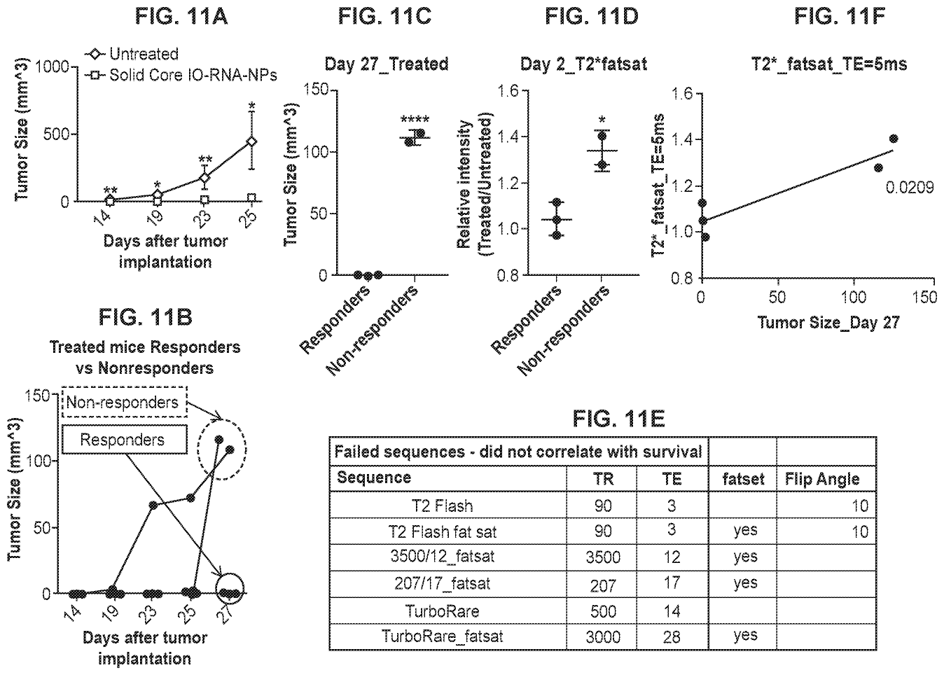

FIG. 11A is a graph of tumor size plotted as a function of days post-tumor implantation in untreated mice or mice treated with IO-RNA-NPs.

FIG. 11B is a graph of the tumor size plotted as a function of days post-tumor implantation in untreated mice or mice treated with IO-RNA-NPs broken into two groups: responders and non-responders.

FIG. 11C is a graph of the tumor size of responders and non-responders on Day 27 in mice treated with IO-RNA-NPs.

FIG. 11D is a graph of the T2* fatsat image intensity of responders and non-responders on Day 2 in mice treated with IO RNA NPs.

FIG. 11E is a table of the sequences that did not correlated with survival.

FIG. 11F is a graph of the Day 2 T2* fatsat image intensity plotted as a function of Day 27 tumor volumes.

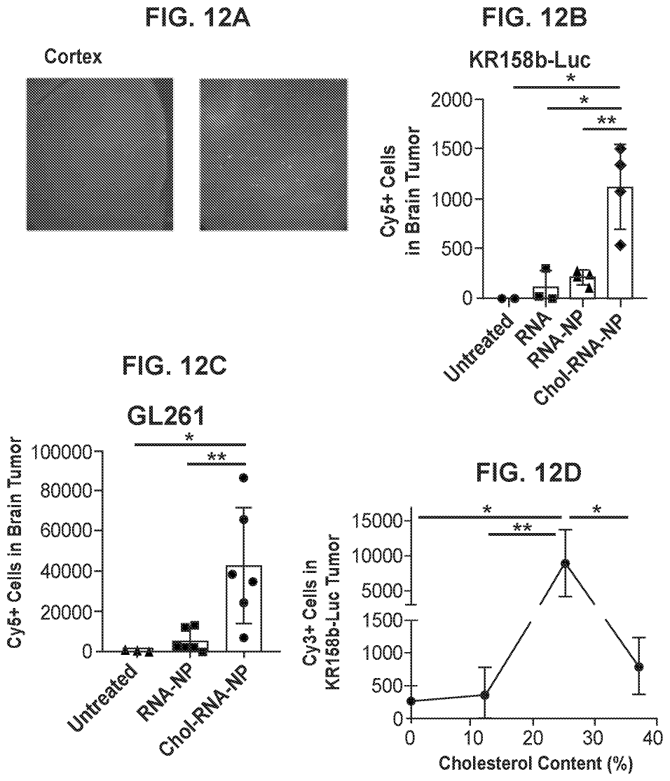

FIGS. 12A-12D show that Chol-RNA-NPs deliver mRNA to brain tumors. FIG. 12A is a representative immunofluorescence microscopy image of KR158b-luciferase tumors 24 hours after injection with Cy3-labelled Chol-RNA-NPs. FIGS. 12B-12C are graphs of flow cytometry-based quantification of Cy5-labelled RNA in intracranial KR158b (FIG. 12B) or GL261 (FIG. 12C) tumors 24 hours after injection of Cy5-labelled liposomes. FIG. 12D is a graph of the RNA delivery to brain tumors after vaccination with RNA-NPs with varying amounts of cholesterol.

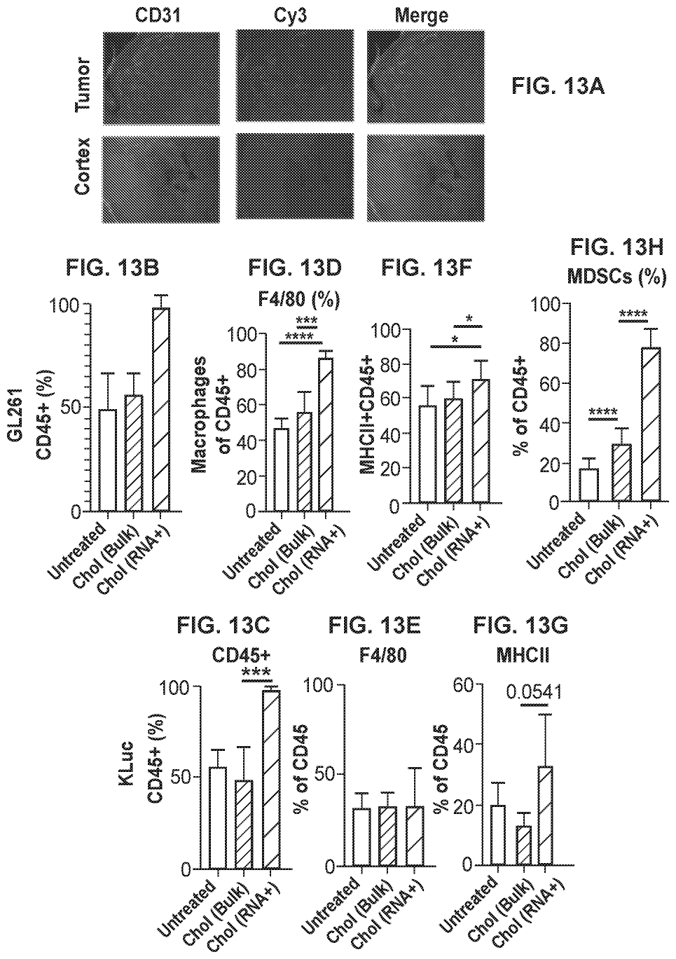

FIGS. 13A-13G show that Chol-RNA-NPs transfect perivascular TAMs. FIG. 13A are representative immunofluorescent images of KR15b tumors and cortex from mice treated with Cy3-labelled Chol-RNA-NPs. FIGS. 13B-13H are graphs of the flow cytometry of tumors with or without vaccination with fluorescently-labelled Chol-RNA-NPs. Data is displayed as percent of total cells that are CD45+(FIG. 13B, 13C), percentage of CD45+ cells that are macrophages (FIGS. 13D-13E), percentage of CD45+ cells that are antigen presenting cells (FIG. 13F-G) and percentage of CD45+ cells that are CD11b+Ly6G/6C+(FIG. 13H) for untreated tumors, treated tumors (Chol (Bulk)), and the RNA+ cells within treated tumors (Chol (RNA+)) for mice with intracranial GL261 (13B, 13D, 13F, 13H) or KR158b-Luciferase (13C, 13E, 13G).

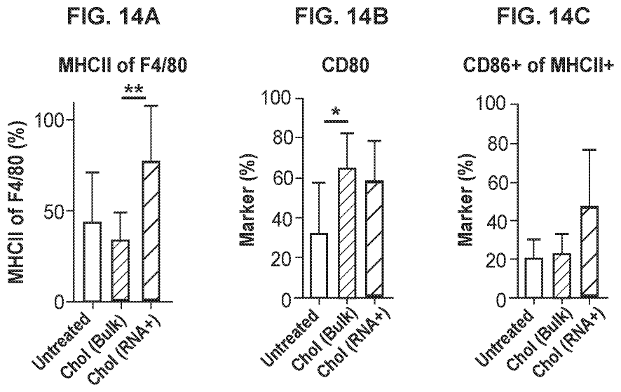

FIGS. 14A-14C show that Chol-RNA-NPs activate CD45+ cells in brain tumors. FIGS. 14A-14D are graphs of Flow cytometry for activation markers on antigen presenting cells in intracranial KR158b tumors 24 hours after vaccination. FIG. 14A, MHCII expression on F4/80+CD45+, FIG. 14B, CD80 expression on MHCII+ CD45+ cells, FIG. 14C, CD86 expression on MHCII+ CD45+ cells.

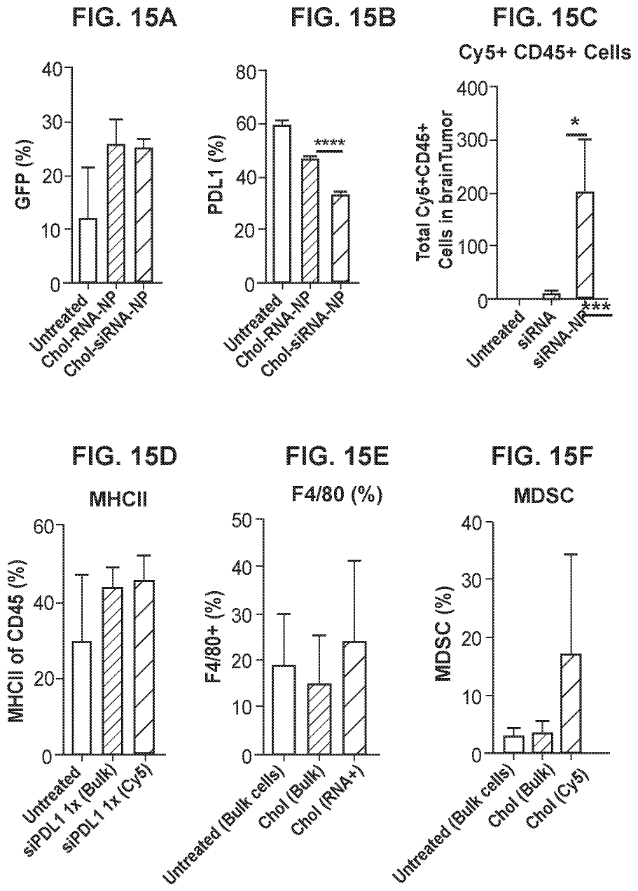

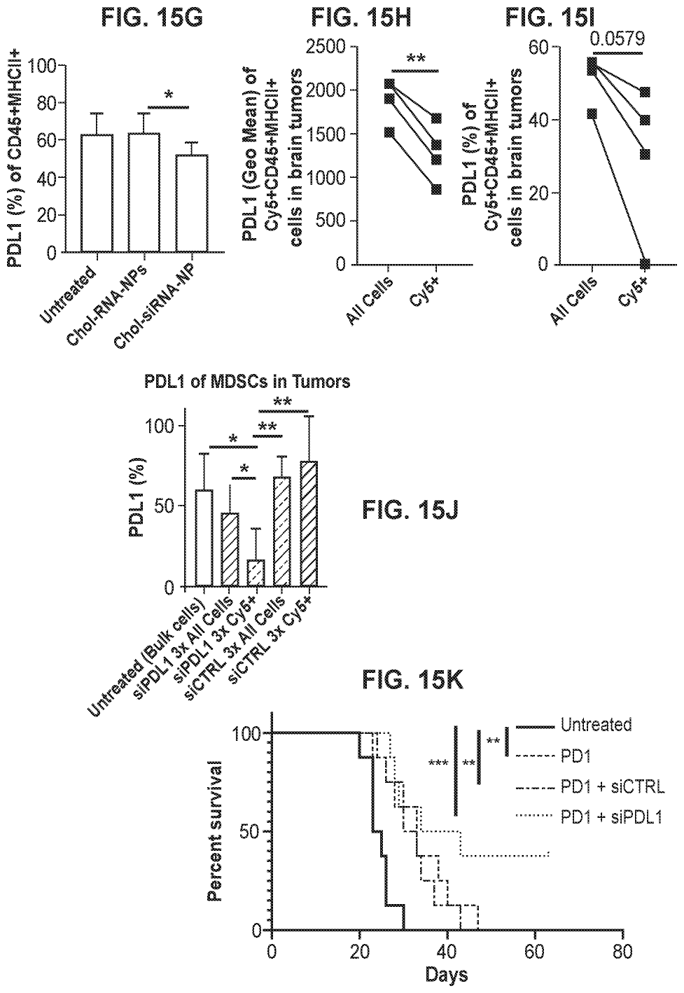

FIGS. 15A-15F show that Chol-RNA-NPs deliver PDL1 siRNA to brain tumors. FIGS. 15A-15B, GFP (15A) and PDL1 (15B) expression 48 hours after transfection of DC2.4s with Chol-RNA-NPs bearing GFP mRNA and siRNA targeting PDL1. FIG. 15C, Uptake of Cy5-labelled PDL1-siRNA in KR158b brain tumors 24 hours after intravenous injection of Chol-RNA-NPs. FIGS. 15D-15F, Flow cytometry to characterize transfected cells by expression of MHCII, F4/80, and CD11 b and Ly6G/6C (MDSCs).

FIGS. 15G-15J show that siPDL1 reduces PDL1 expression in KLuc brain tumors. FIGS. 15G-151, PDL1 expression on CD45+MHCII+ cells 24 hours after vaccination with Chol-RNA-NPs bearing siPDL1. FIG. 15J, PDL1 expression on CD11b+Ly6G/6C+ cells (MDSCs) in intracranial KR158b-Luciferase tumors 24 hours after the last of three daily vaccinations with Chol-RNA-NPs bearing siPDL1.

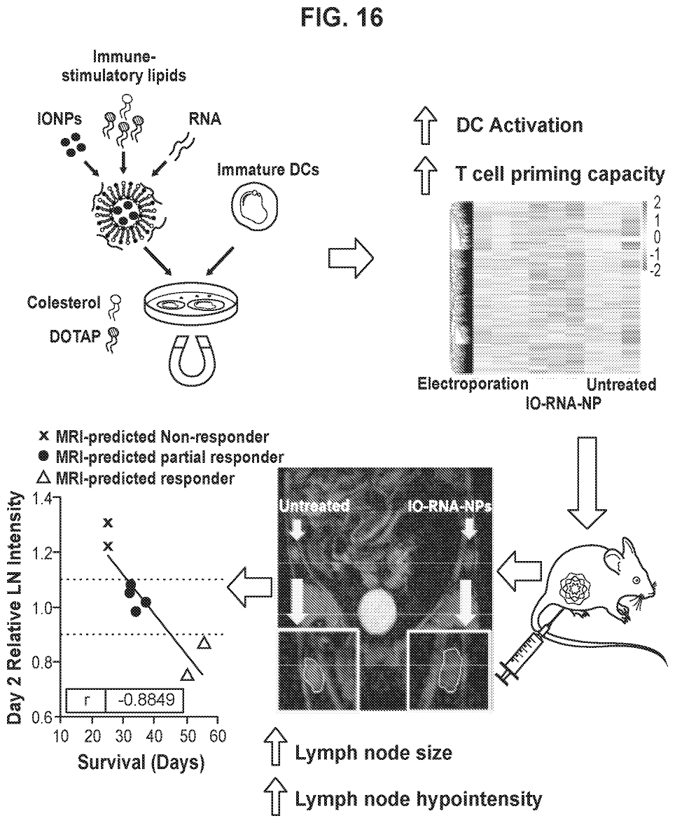

FIG. 16 is a schematic showing IO-RNA-NPs were generated by combining commercially available IONPs and mRNA encoding tumor antigens with a combination of previously translated lipids with exceptional capacity for mRNA delivery and DC activation. Incubation of these particles with DCs in the presence or absence of a magnetic field led to profound DC activation characterized by dramatic changes in RNA expression and enhanced capacity to stimulate antigen specific T cells. IO-RNA-NPs enabled MRI-based detection of DC migration to lymph nodes that correlated directly with survival in murine tumor models.

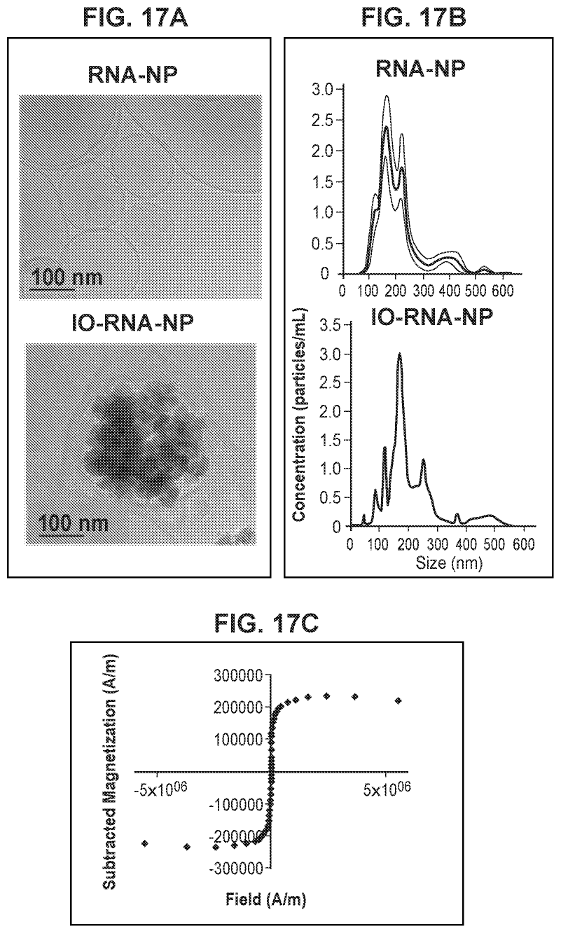

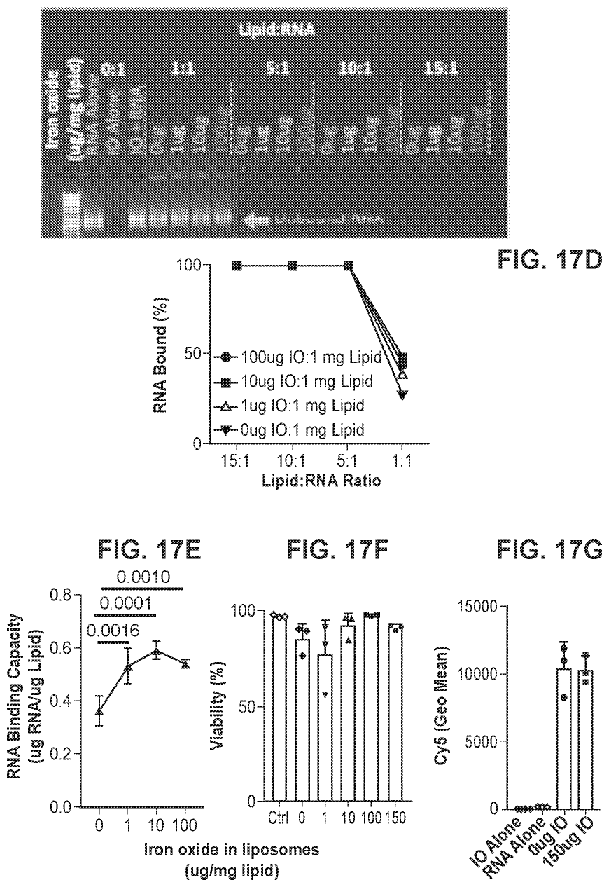

FIGS. 17A-17G show the development and characterization of iron oxide loaded RNA-nanoparticles. FIG. 17A, Representative Cryo-TEM of RNA-NPs with or without iron oxide (IO). FIG. 17B, Size distribution of RNA-NPs with and without IO (100 ug IO:1 mg lipid) assessed by Nanosight. FIG. 17C, Saturation magnetization of IO-RNA-NPs (100 ug IO:1 mg lipid) assessed with a SQUID magnetometer. FIG. 17D, Agarose gel electropheresis demonstrating RNA bound by different formulations of IO-RNA-NPs (labelled as the mass of IONPs in each formulation per mg lipid) after 15 minute incubation with RNA at different lipid:RNA ratios. FIG. 17E, RNA-binding capacity for RNA-NPs containing varying amounts of iron oxide. Numbers on graph are p values derived from one-way ANOVA and Tukey's tests with n=4 per group. FIG. 17F, Viability of DC2.4s after 24 hour incubation with RNA-NPs assessed by flow cytometry (n=3). FIG. 17G, Uptake of Cy5-labelled RNA by DC2.4s assessed by flow cytometry after overnight incubation with RNA-NPs (n=3).

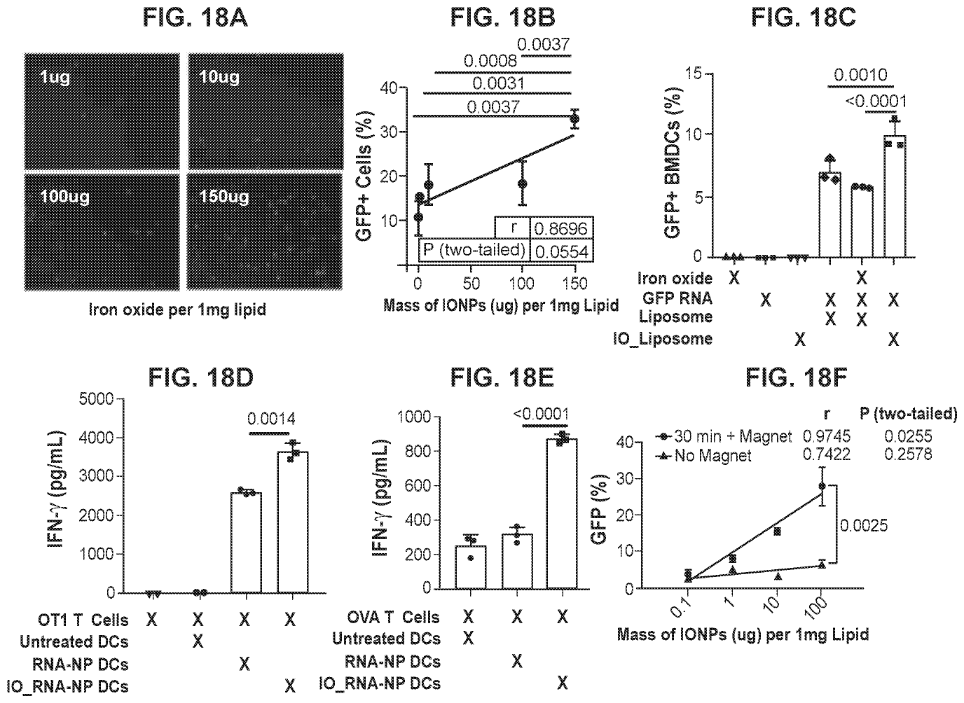

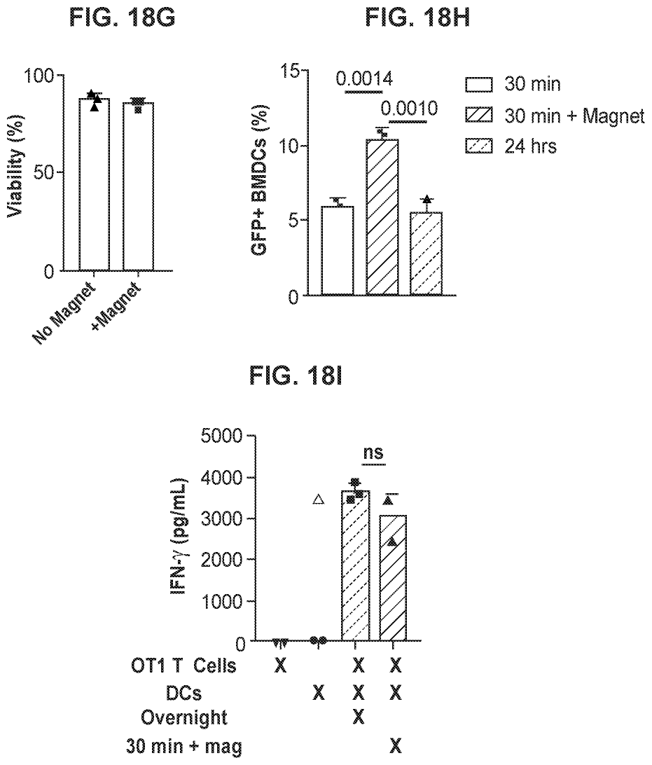

FIGS. 18A-18I show that iron oxide enhances transfection and activation of dendritic cells. FIG. 18A, Representative images of GFP expression in DC2.4s after 24-hour incubation with RNA-NPs synthesized with varying amounts of iron oxide per 1 mg lipid. FIG. 18B, Quantification of transfection efficiency from (a) with flow cytometry. A Pearson's correlation and an ANOVA with Tukey's tests were used for statistical analysis. FIG. 18C, Fluorescence in BMDCs after transfection with GFP-RNA-loaded liposomes with no iron oxide, iron oxide encapsulated inside the liposomes (IO-Liposome), or iron oxide added to the media outside the liposomes. An ANOVA with Tukey's tests were used for statistical analysis. FIGS. 18D-18E, ELISA for IFN-gamma produced after a two day co-culture combining DCs loaded with OVA RNA via RNA-NPs or IO-RNA-NPs with naïve splenocytes from OT1 mice (d) or antigen experienced OVA T-cells (e). FIG. 18F, Transfection efficiency in DC2.4s after a 30 minute incubation with IO-RNA-NPs in the presence or absence of a magnetic field. A Pearson's correlation coefficient was used for statistical analysis. FIG. 18G, Viability of BMDCs 24 hours after a 30 minute incubation with IO-RNA-NPs in the presence or absence of a magnetic field. FIG. 18H, GFP expression in BMDCs after either a 30 minute incubation with IO-RNA-NPs (100 ug IONP:1 mg lipid) in the presence or absence of a magnetic field or an overnight incubation in the absence of a field. One-way ANOVA with Tukey's tests were used for statistical analysis. FIG. 18I, ELISA for IFN-γ produced during a two day co-culture of antigen-naïve OT1 T-cells and BMDCs treated with IO-RNA-NPs bearing OVA mRNA either overnight or for 30 minutes in the presence of a magnetic field. For all experiments, n=3, error bars represent standard deviation from the mean, and numbers are P values calculated from two-tailed unpaired two sample t tests or Tukey's tests as appropriate. Results in 18A-C, and 18F-I are each representative of at least two replicate experiments. ns=not significant.

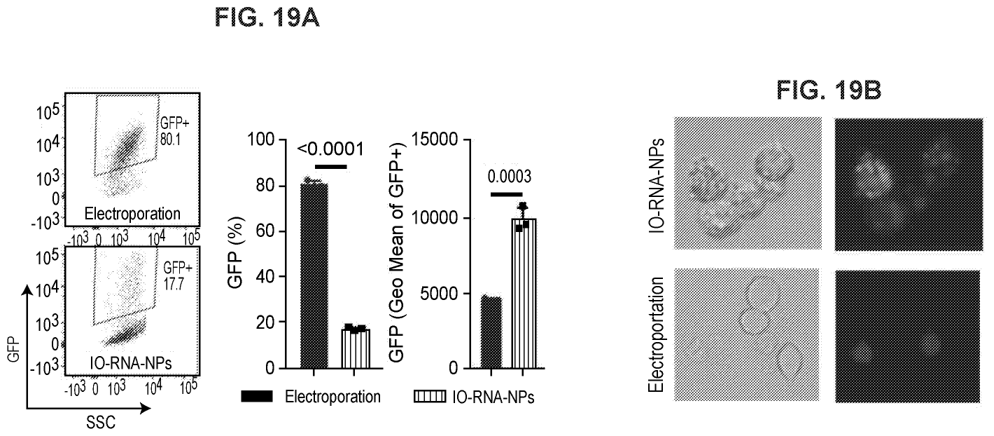

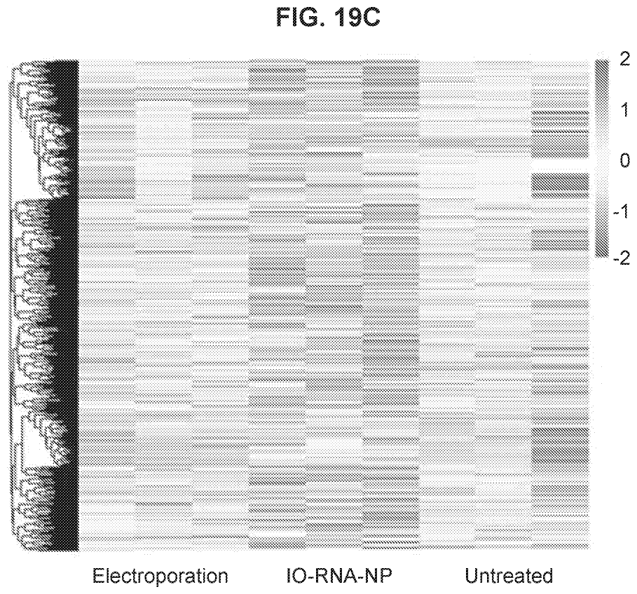

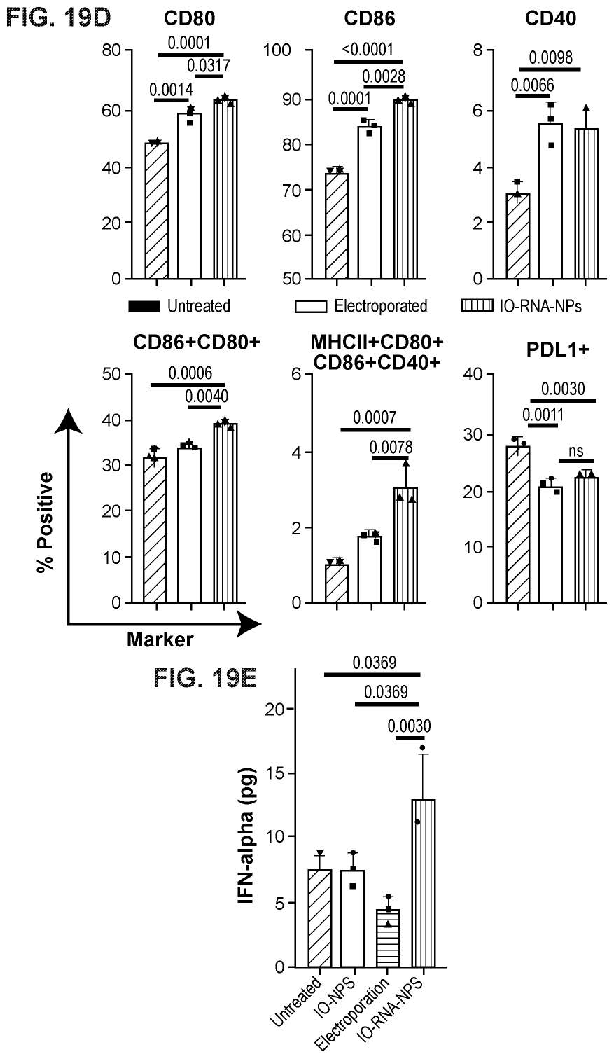

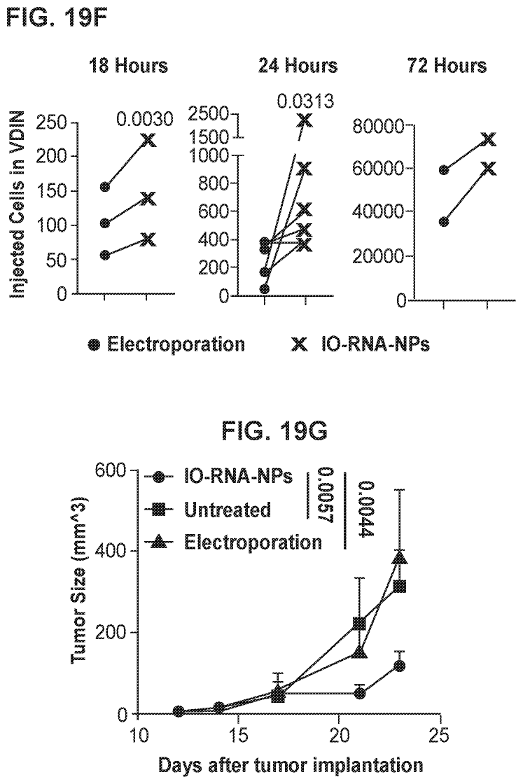

FIGS. 19A-19G show that IO-RNA-NPs enhance DC activation and migration compared to electroporation. FIG. 19A, Representative flow cytometry plots (left), transfection efficiency (center) and geometric mean fluorescence intensity (right) 24 hours after transfection of BMDCs with GFP RNA via electroporation or IO-RNA-NPs (n=3). FIG. 19B, Fluorescent microscope images of DC2.4s incubated overnight with Cy3-labelled RNA-NPs. FIG. 19C, Heat map comparing RNA expression in BMDCs 24 hours after treatment with IO-RNA-NPs, Electroporation, or media alone. FIG. 19D-19E, Phenotypic markers of activation assessed by flow cytometry (FIG. 19D) and IFN-alpha release assessed by ELISA (FIG. 19E) for BMDCs 24 hours after treatment with electroporation or IO-RNA-NPs. One-way ANOVA and Tukey's tests were used for statistical analysis. FIG. 19F, Migration of JO-RNA-NP-loaded BMDCs to VDLN at varying timepoints after intradermal injection. Statistical analysis was completed with Wilcoxon matched-pairs rank sum test for n>4 or student's paired t test for n<4. FIG. 19G, Tumor growth in mice (Untreated: n=6; Electroporation: n=6; IO-RNA-NPs: n=13) with subcutaneous B16F10-OVA tumors after a single vaccination with 500,000 BMDCs pulsed with OVA mRNA via electroporation or IO-RNA-NPs and 10 million naïve OT1 T-cells. Data is pooled from two independent experiments. A two-way ANOVA was used for statistical analysis. Results in FIGS. 19A, 19B, and 19D-19G, are each representative of at least 2 replicate experiments. Numbers on graphs are P values

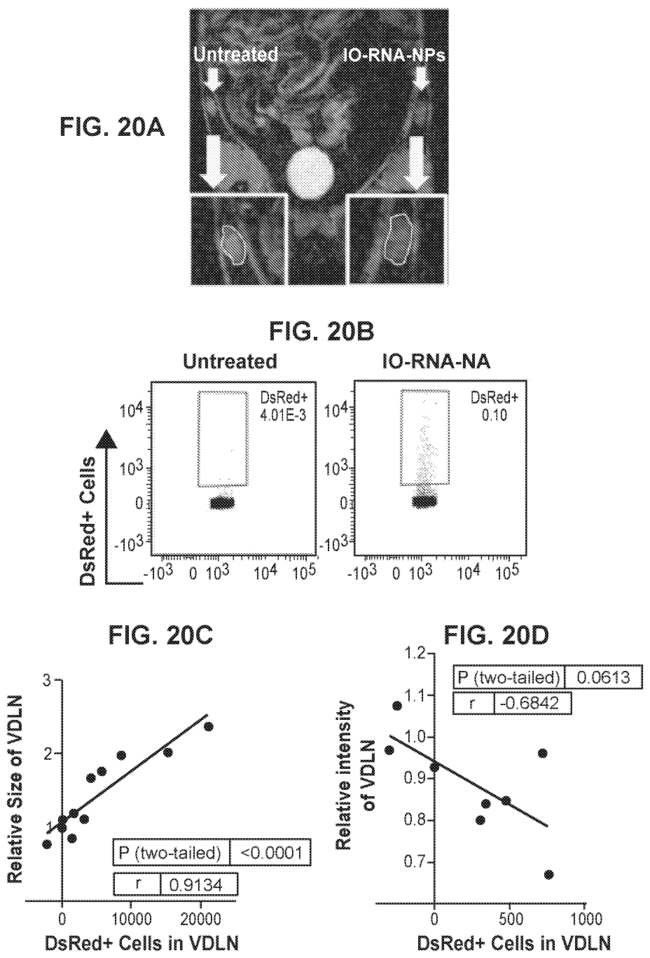

FIGS. 20A-20D show that IO-RNA-NPs enable quantitative cell tracking with MRI. FIG. 20A, T2*-weighted MRI image 48 hours after vaccination with IO-RNA-NP-loaded DsRed+ DCs in the left inguinal area. Yellow borders indicate lymph nodes on treated (right) and untreated (left) sides. FIG. 20B, Exemplary flow cytometry plots demonstrating gating on DsRed+ cells in lymph nodes. FIG. 20C, Correlation of relative lymph node size between treated and untreated lymph nodes and the absolute count of DCs in that lymph node. Data is combined from 2 independent experiments. FIG. 20D, Correlation of relative T2*-weighted MRI intensity in treated and untreated lymph nodes with absolute counts of labelled cells. Data is representative of two replicate experiments. p values and r values are derived from a Pearson correlation.

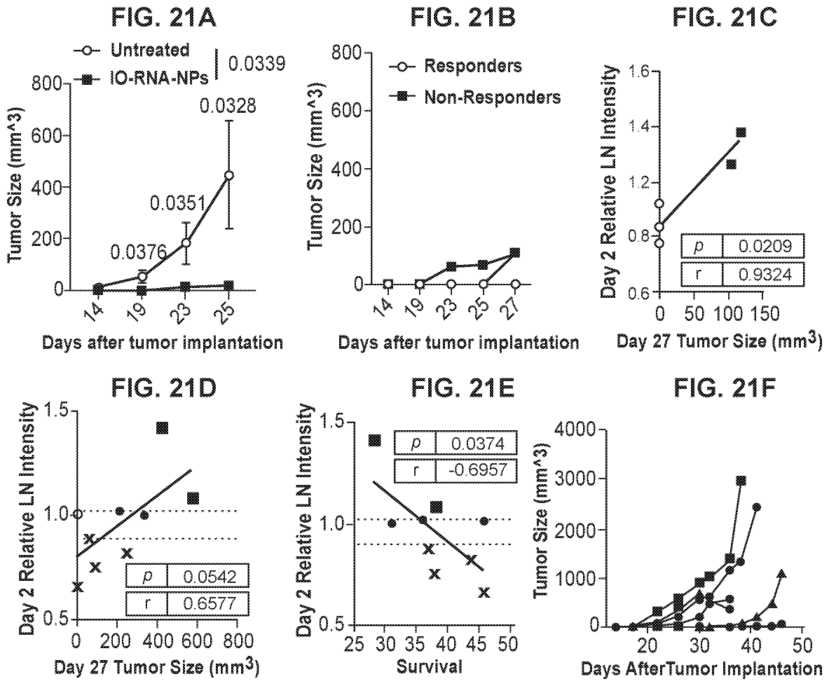

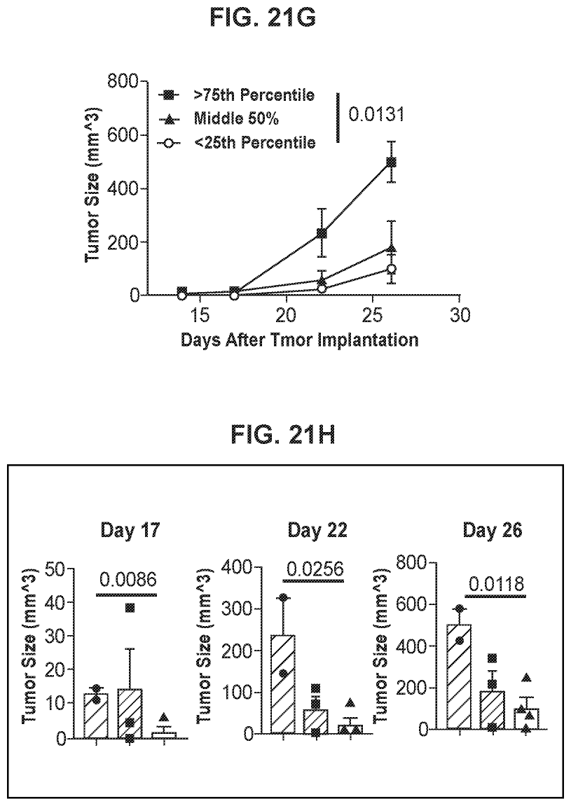

FIGS. 21A-21J show MRI-detected DC migration predicts response to DC vaccines. Mice with subcutaneous B16F10-OVA tumors were treated with BMDCs loaded with IO-RNA-NPs bearing ovalbumin mRNA. FIG. 21A, Tumor growth over time between treated mice (n=7) and untreated mice (n=5). Numbers on graph are P values calculated by unpaired student's t tests. Comparison of tumor growth over time is evaluated with a two-way ANOVA. FIG. 21B, Growth of individual treated tumors separated into “responders” and “non-responders”. FIG. 21C, Correlation of the relative change in MRI-detected lymph node intensity in treated compared to untreated lymph nodes (Relative LN Intensity) on Day 2 with Day 27 tumor size. Dotted lines demarcate the 25th and 75th percentiles of relative MRI intensity in lymph nodes. Datapoints from mice with substantial MRI-predicted DC migration indicated by relative VDLN intensity in the bottom 25th percentile are X's in FIGS. 21E and 21E, those from mice with moderate VDLN intensity in the middle 50th percentile are dots in FIGS. 21D and 21E, and those from mice with high VDLN intensity in the top 75th percentile are squares in FIGS. 21D and 21E. FIGS. 21D-21E Correlation of Day 2 MRI-detected DC migration with tumor size (d) and survival (n=10) (e). Numbers on graphs c-e represent Pearson's correlation coefficient (r) and P value (p). FIGS. 21F-21H, Individual tumor growth curves (FIG. 21F), summary data (FIG. 21G), and tumor sizes at multiple timepoints separated by MRI intensity on Day 2 after vaccination. Numbers on graphs are p values calculated from an ANOVA (FIG. 21G) and two-tailed unpaired t tests (FIG. 21H).

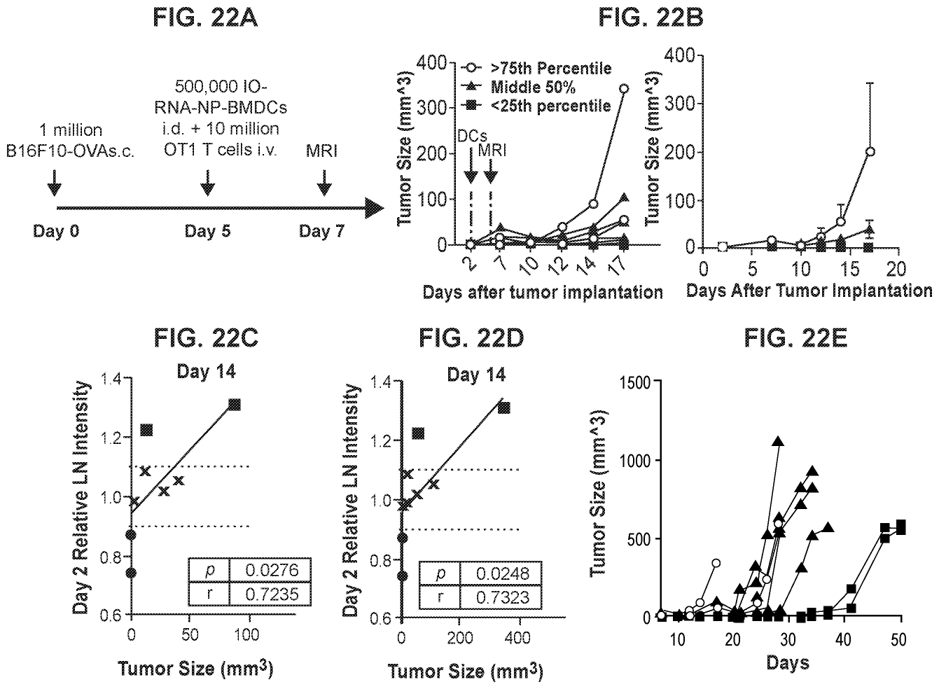

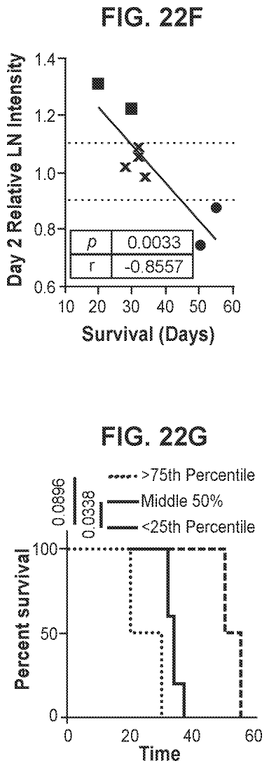

FIGS. 22A-22G show that MRI-detected DC migration at two days post-vaccine predicts antitumor efficacy of therapeutic DC vaccine. FIG. 22A, Schematic of treatment schedule. Mice received subcutaneous injection of 1 million B16F10-OVA cells in the lateral flank. On Day 5 mice received intradermal (i.d.) injection of 500,000 BMDCs loaded with 10-RNA-NPs bearing OVA RNA and intravenous (i.v.) injection of 10 million OT1 T-cells. Mice were imaged with MRI after two days and followed for tumor growth and survival. FIG. 22B, Individual tumor growth curves (left) and summary data (right) for all treated mice before deaths (n=9). FIGS. 22C-22D, Correlation of T2*-weighted MRI intensity on Day 2 with tumor size on Day 14 (c) and Day 17 (FIG. 22D). Dotted lines demarcate the 25th and 75th percentiles of relative MRI intensity in lymph nodes. FIG. 22E, Individual tumor growth curves (left) and summary data (right) through deaths of all treated mice. Numbers on the graph are p values calculated using two-way ANOVA tests for significance. FIG. 22F, Correlation of T2*-weighted MRI intensity on Day 2 with Survival. FIG. 22G, Survival curves for all treated mice. Numbers on graph are p values calculated using a Log-Rank test. P and r values in FIGS. 22C, 22D, and 22F are derived from a Pearson Correlation.

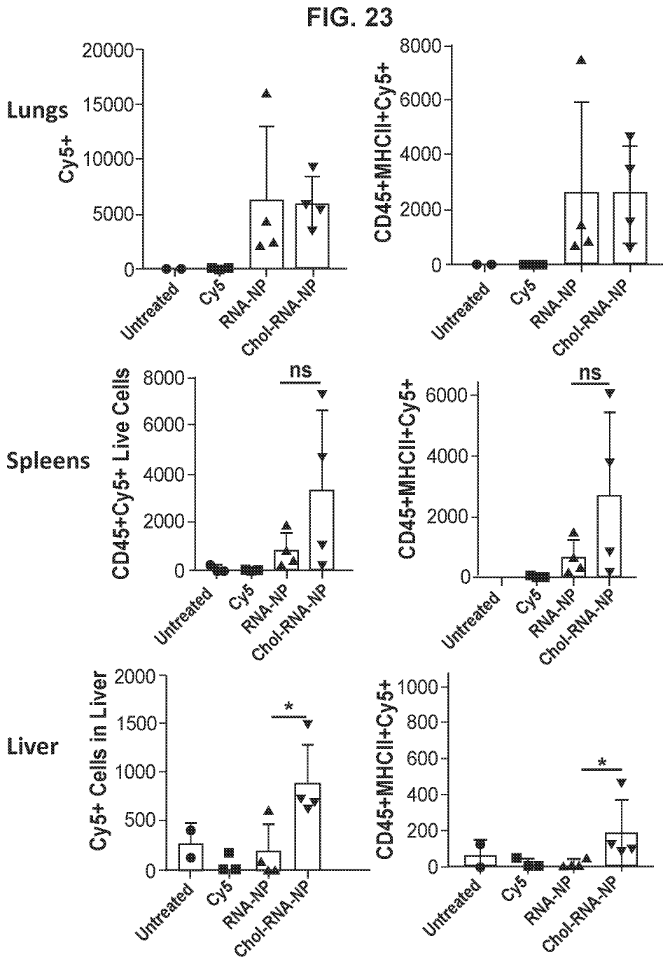

FIG. 23 is a series of graphs of Cy5+ cells (left column) or CD45+MHC Class II+Cy5+ cells (right column) of untreated mice or mice treated with Chol-RNA-NP, RNA-NP, or Cy5-labeled RNA only as detected in the lungs (top row), spleens (middle row), or liver (bottom row).

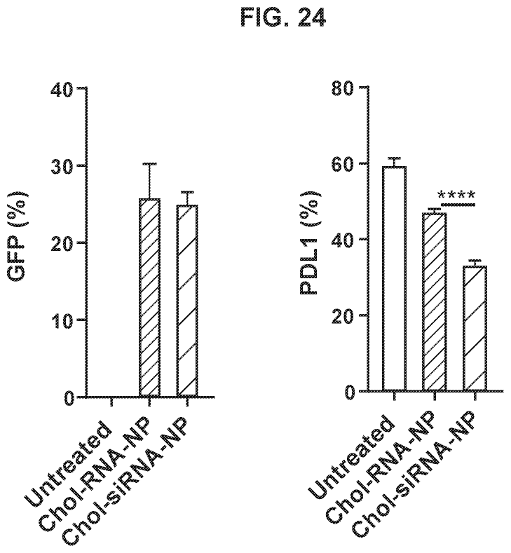

FIG. 24 is a pair of graphs of % GFP (left) or % PDL1 (right) for untreated mice or mice treated with Chol-RNA-NP or Chol-siRNA-NP.

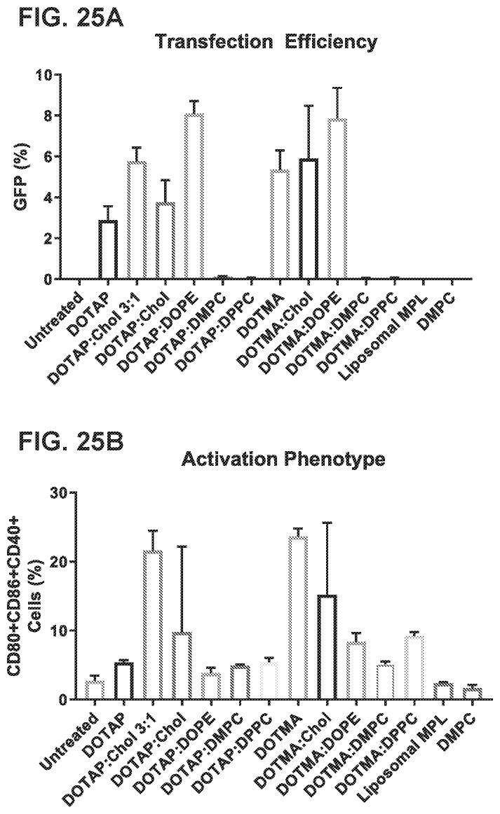

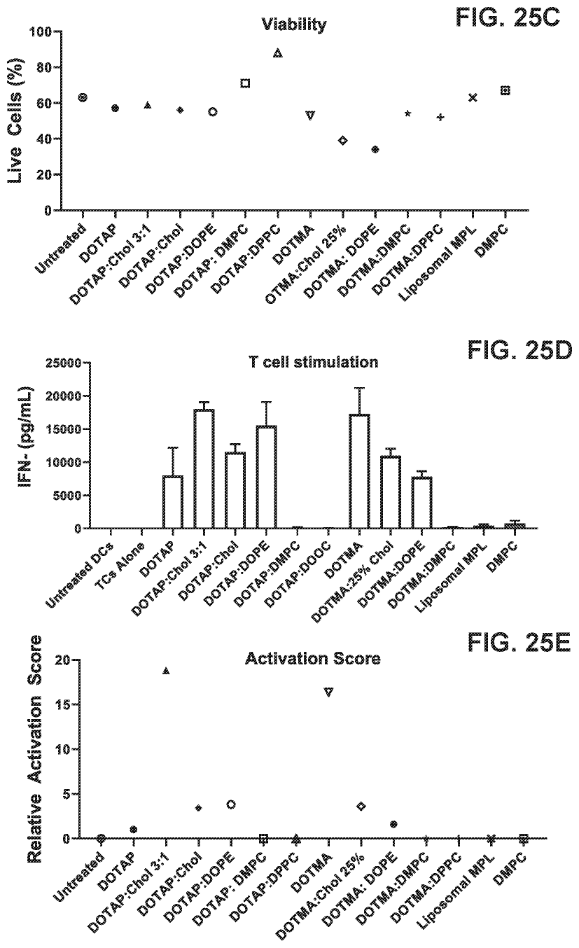

FIGS. 25A-25E show translatable nanoparticles transfect and activate DCs. FIG. 25A, GFP expression in BMDCs after 24 hour incubation with each RNA-loaded particle. FIG. 25B, Co-expression of CD40, CD80, and CD86 on BMDCs after 24 hour incubation with each particle construct. FIG. 25C, Viability of BMDCs after 24 hour incubation with each construct. FIG. 25D, BMDCs were loaded with IO-RNA-NPs bearing OVA RNA and incubated with OVA-specific OT1 T cells. T cell activation is displayed as IFN-γ assessed by ELISA. FIG. 25E, Relative Activation Score calculated as the product of average values for Viability, Transfection Efficiency, Activation, and T cell Stimulation for each formulation normalized to the score for DOTAP.

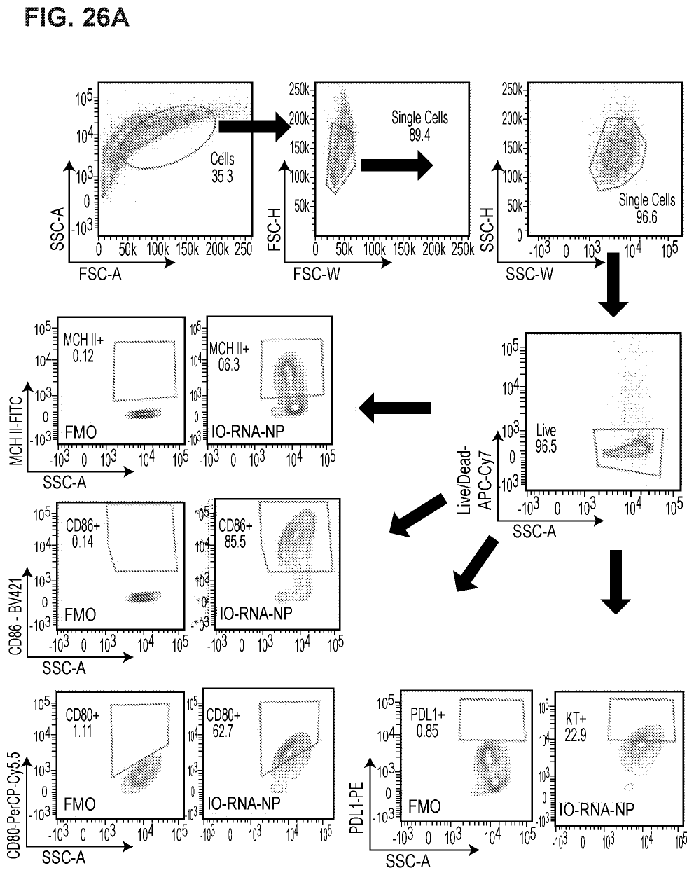

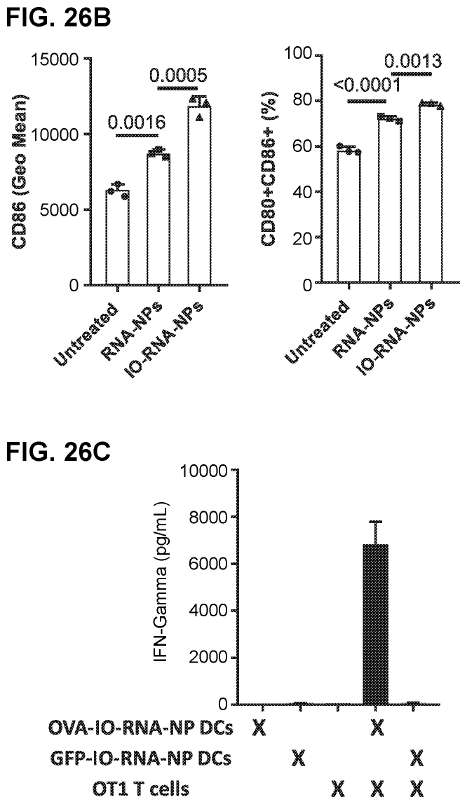

FIGS. 26A-26C show that IO-RNA-NPs induce antigen specific DC activation. FIG. 26A, Gating Strategy. Positive populations were selected using fluorescence minus one (FMO) controls. FIG. 26B, Expression of CD86 and co-expression of CD80 and CD86 on BMDCs after 24 hour incubation with RNA-NPs or IO-RNA-NPs. Data are plotted as geometric mean fluorescence intensity and percentages. One-way ANOVA and Tukey's tests were used for statistical comparisons. Numbers on graphs are P values. FIG. 26C, BMDCs were loaded with IO-RNA-NPs bearing either OVA RNA or GFP RNA and incubated with OVA-specific OT1 T cells. T cell activation is displayed as IFN-γ assessed by ELISA.

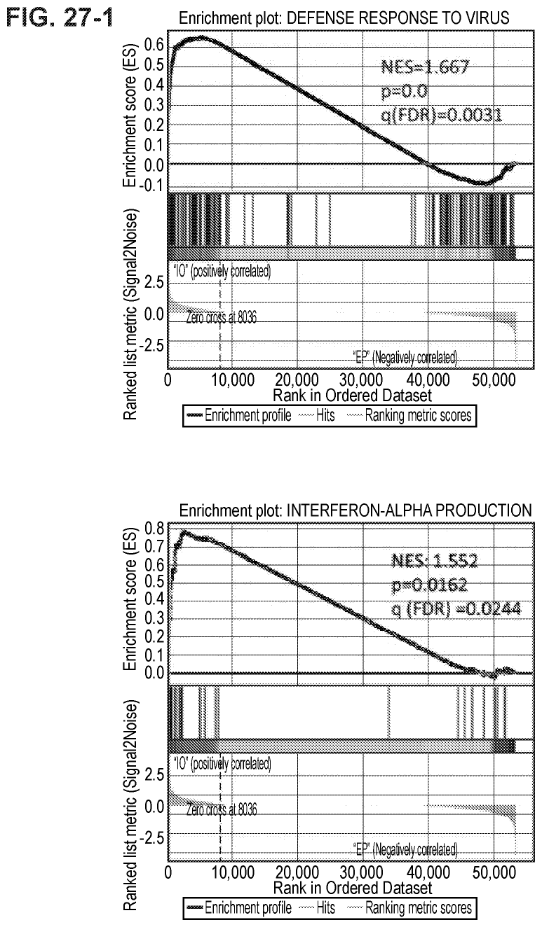

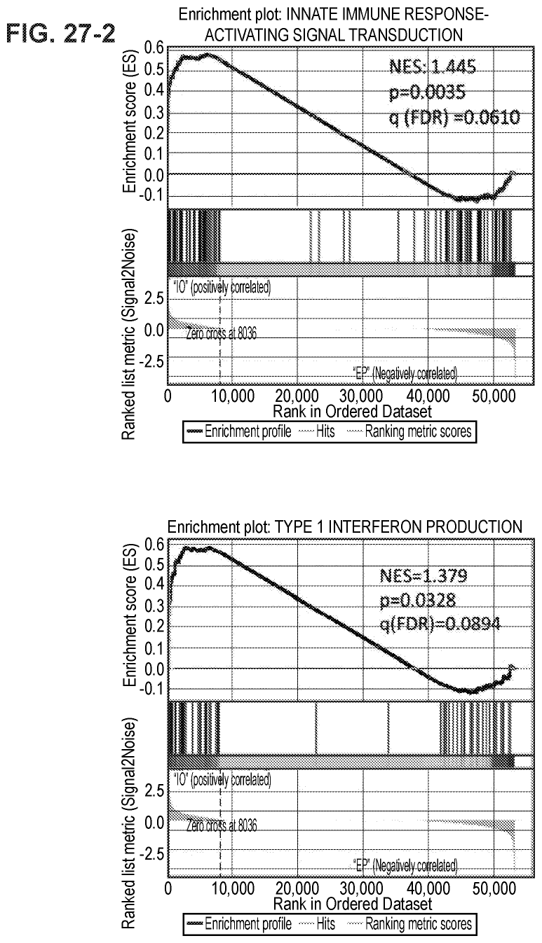

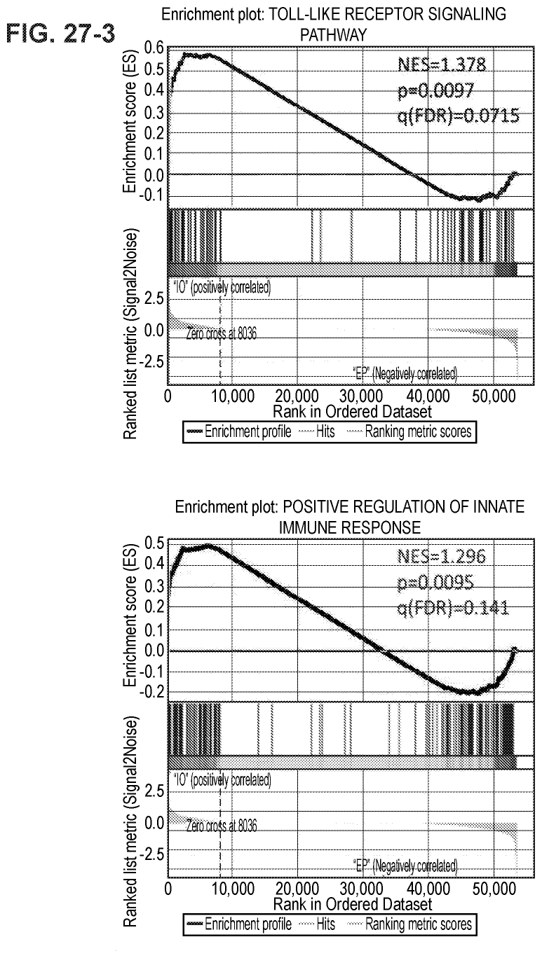

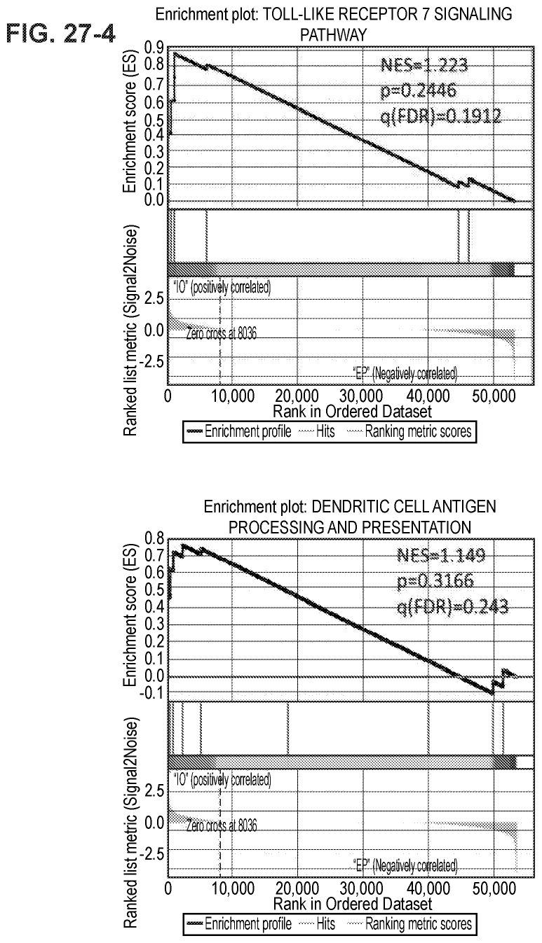

FIG. 27 shows that IO-RNA-NPs induce expression of antiviral gene sets. Gene set enrichment plots for 8 gene sets relating to RNA uptake and processing in primary BMDCs 24 hours after treatment with GFP mRNA via either IO-RNA-NPs or electroporation.

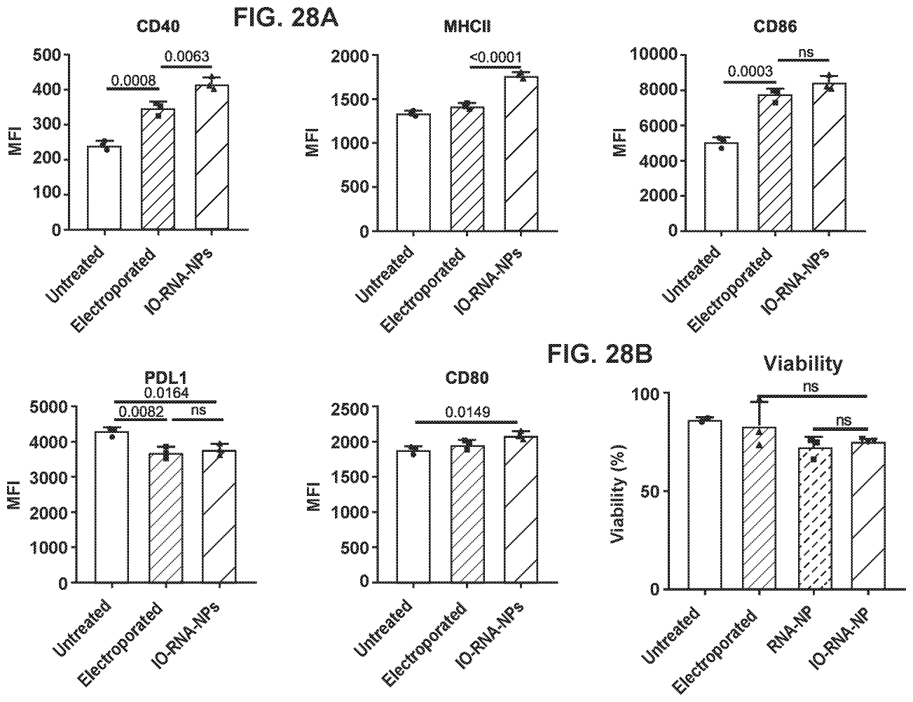

FIGS. 28A-28B show that IO-RNA-NPs enhance activation of BMDCs. FIG. 28A, Phenotypic markers of activation on BMDCs assessed by flow cytometry 24 hours after treatment with electroporation or IO-RNA-NPs. Data are presented as geometric mean fluorescence intensity. One-way ANOVA and Tukey's tests were used for statistical analysis. FIG. 28B BMDC viability 24 hours after electroporation or incubation with RNA-NPs or IO-RNA-NPs as assessed by automated cell counter.

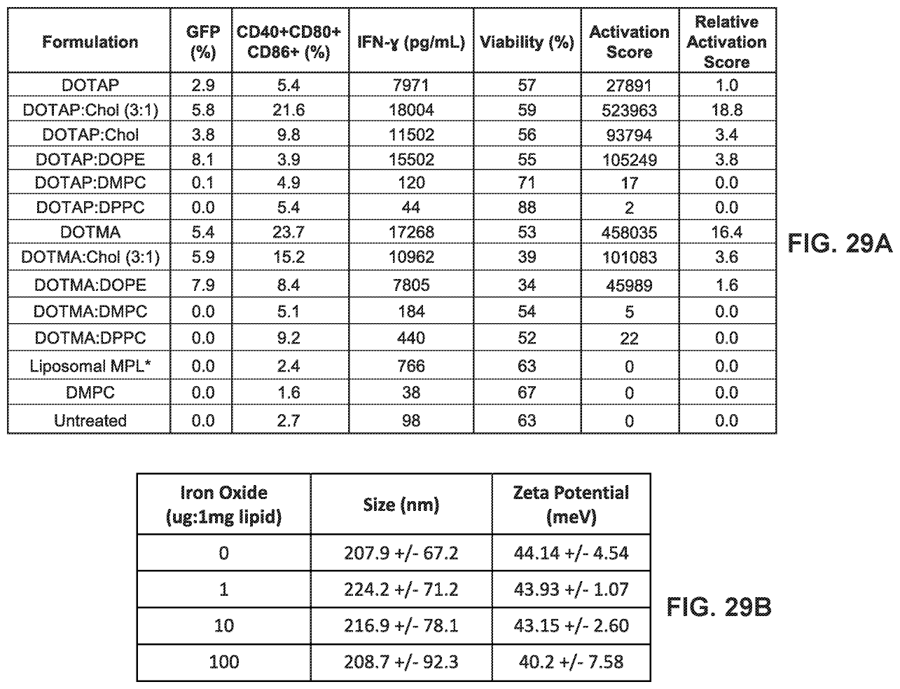

FIG. 29A is a table showing summary data for lipid library. Liposomes were formed from the listed components at a 1:1 ratio unless otherwise noted. Summary data derived at 24 hours after incubation with BMDCs with GFP mRNA or 48 hours after incubation of OVA-transfected BMDCs with OVA-specific OT1 T cells are shown for each particle construct. Activation Score is calculated as the product of transfected cells (GFP (%)), activated cells (CD40+CD80+CD86+(%)), viability (%), and T cell stimulating capacity (IFN-γ (pg/mL)). Relative Activation Scores are calculated as (Activation ScoreSample)/(Activation ScoreDOTAP). DOTAP=1,2-dioleoyl-3-trimethylammonium-propane; Chol=Cholesterol; DOPE=1,2-dioleoyl-sn-glycero-3-phosphoethanolamine; DMPC=1,2-dimyristoyl-sn-glycero-3-phosphocholine; DPPC=1,2-dipalmitoyl-sn-glycero-3-phosphocholine; DOTMA=1,2-di-O-octadecenyl-3-trimethylammonium propane. *Liposomal MPL is a mixture of lipids and the adjuvant monophosphoryl lipid A (MLA) at a ratio of Cholesterol:DMPG:DPPC:MLA of 5.2:1.1:8.7:0.15.

FIG. 29B is a table showing the characterization of iron oxide loaded RNA-nanoparticles. Zeta potential and size (measured by Nanosight) are displayed as the mean+/−the standard deviation. Results are representative summary data of three repeated experiments for each zeta potential and 4 measurements for each size. Size measurements were completed with RNA bound to liposomes. Zeta potential was measured for liposomes without RNA.

### DETAILED DESCRIPTION

Presented herein for the first time are data which culminates in the development of bi-functional RNA-loaded nanoparticles (RNA-NPs) that enhance DC migration to lymph nodes and track migration in vivo using a widely available MRI-based imaging modality. As described herein, cationic liposomes with iron oxide nanoparticle cores were incubated with mRNA. The resulting iron oxide-loaded RNA-NPs (IO-RNA-NPs) were used to transfect DsRed⁺ DCs ex vivo in the presence of a magnetic field. IO-RNA-NP-loaded DCs were then injected intradermally into C57B16 mice and tracked noninvasively with T2*-weighted 11T MRI. MRI intensity was correlated with Prussian blue staining for iron oxide content and flow cytometry for absolute counts of DsRed+ cells in each lymph node. The presence of iron oxide in RNA-NPs did not significantly modify particle characteristics including size, charge, RNA-binding capacity, and transfection of DCs. Additionally, inclusion of iron oxide within RNA-NPs enabled magnetically enhanced RNA delivery and transfection efficiency through application of external magnetic fields. Compared to RNA electroporation, IO-RNA-NP loading enhanced production of antiviral cytokines (IFN-alpha) and DC migration to lymph nodes. IO-RNA-NPs also produced a reduction in T2*-weighted MRI intensity and an increase in MRI-detected lymph node size that correlated directly with the number of iron oxide loaded cells in treated lymph nodes. T2*-weighted MRI intensity measured two days after vaccination correlated with inhibition of tumor growth in murine tumor models. These data suggest that IO-RNA-NPs enhance DC activation and allow noninvasive cell tracking with MRI. Without being bound to any particular theory, these data support the use of IO-RNA-NPs for predicting antitumor immune responses and for using MRI-detected DC migration as a biomarker for vaccine efficacy.

Liposomes, Cells, & Compositions

The present disclosure provides a liposome comprising ribonucleic acid (RNA) molecules and a lipid mixture comprising DOTAP and cholesterol. In various embodiments, the liposome comprises RNA molecules, a lipid mixture comprising DOTAP and cholesterol, and iron oxide nanoparticles (IONPs), optionally, wherein the DOTAP and cholesterol are present in the lipid mixture at a DOTAP:cholesterol ratio of about 3:1 by mass. In various embodiments, the presently disclosed liposome comprises RNA molecules and a lipid mixture comprising DOTAP and cholesterol, wherein the DOTAP and cholesterol are present in the lipid mixture at a DOTAP:cholesterol ratio of about 3:1 by mass. As used herein, the term “DOTAP” means N-(2,3-Dioleoyloxy-1-propyl)trimethylammonium methyl sulfate.

In exemplary aspects, the liposome has a diameter between about 80 nm to about 500 nm, e.g., about 80 nm to about 450 nm, about 80 nm to about 400 nm, about 80 nm to about 350 nm, about 80 nm to about 300 nm, about 80 nm to about 250 nm, about 80 nm to about 200 nm, about 90 nm to about 500 nm, about 100 nm to about 500 nm, about 150 nm to about 500 nm, about 200 nm to about 500 nm, about 250 nm to about 500 nm, about 300 nm to about 500 nm, about 350 nm to about 500 nm, about 400 nm to about 500 nm. In exemplary aspects, the liposome has a diameter between about 90 nm to about 300 nm, e.g., about 100 nm to about 250 nm, about 110 nm±5 nm, about 115 nm±5 nm, about 120 nm±5 nm, about 125 nm±5 nm, about 130 nm±5 nm, about 135 nm±5 nm, about 140 nm±5 nm, about 145 nm±5 nm, about 150 nm±5 nm, about 155 nm±5 nm, about 160 nm±5 nm, about 165 nm±5 nm, about 170 nm±5 nm, about 175 nm±5 nm, about 180 nm±5 nm, about 190 nm±5 nm, about 200 nm±5 nm, about 210 nm±5 nm, about 220 nm±5 nm, about 230 nm±5 nm, about 240 nm±5 nm, about 250 nm±5 nm, about 260 nm±5 nm, about 270 nm±5 nm, about 280 nm±5 nm, about 290 nm±5 nm, about 300 nm±5 nm. In exemplary aspects, the liposome has an overall surface net charge of about 20 mV to about 50 mV (e.g., 20 mV to about 45 mV, about 20 mV to about 40 mV, about 20 mV to about 35 mV, about 20 mV to about 30 mV, about 20 mV to about 25 mV, about 25 mV to about 50 mV, about 30 mV to about 50 mV, about 35 mV to about 50 mV, about 40 mV to about 50 mV, or about 45 mV to about 50 mV. In exemplary aspects, the liposome has an overall surface net charge of about 40 mV to about 50 mV.

In exemplary aspects, the mass of the cholesterol is more than 12% and less than 37% of the total lipid mass of the lipid mixture of the liposome of the present disclosure. For example, the mass of the cholesterol is more than 15% and less than 35% of the total lipid mass. In exemplary aspects, the mass of the cholesterol is about 15% to about 30% of the total lipid mass of the lipid mixture. In exemplary aspects, the mass of the cholesterol is about 20% to about 30% of the total lipid mass of the lipid mixture. In exemplary aspects, the mass of the cholesterol is about 25%±3% of the total lipid mass of the lipid mixture. In exemplary aspects, the mass of the DOTAP is at least 50% of the total lipid mass of the lipid mixture. For example, the mass of the DOTAP is about 63% to about 88% of the total lipid mass of the lipid mixture. In exemplary instances, the mass of the DOTAP is about 75%±5% of the total lipid mass of the lipid mixture. In some aspects, when the lipid mixture comprises a third lipid which is different from DOTAP and cholesterol, the mass of the third lipid is less than about 10% of the total lipid mass of the lipid mixture, optionally, less than about 5%, less than about 4%, less than about 3%, less than about 2%, or less than about 1%, of the total lipid mass of the lipid mixture. In certain aspects, the lipid mixture consists essentially of DOTAP and cholesterol.

In exemplary aspects, the liposome comprises RNA molecules (or “RNA”) which can be single-stranded or double-stranded, synthesized or obtained (e.g., isolated and/or purified) from natural sources, which can contain natural, non-natural or altered nucleotides, and which can contain a natural, non-natural or altered inter-nucleotide linkage. In exemplary aspects, the nucleic acid molecule comprises one or more modified nucleotides, such as, e.g., 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridme, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N-substituted adenine, 7-methylguanine, 5-methylammomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouratil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. In exemplary aspects, the RNA comprises one or more non-natural or altered inter-nucleotide linkages, such as a phosphoroamidate linkage or a phosphorothioate linkage, in place of the phosphodiester linkage found between the nucleotides of a naturally-occurring DNA molecule or RNA molecule. In exemplary aspects, the RNA does not comprise any insertions, deletions, inversions, and/or substitutions. However, it may be suitable in some instances, as discussed herein, for the RNA to comprise one or more insertions, deletions, inversions, and/or substitutions. In some aspects, the RNA molecule is a mature mRNA or a processed mRNA that lacks introns. In exemplary aspects, the RNA molecule comprises a 5′ cap, a poly(A) tail, or a combination of both. The 5′ cap in various aspects comprises a 7-methylguanylate and is attached to the 5′ end of the RNA molecule via a 5′ to 5′ triphosphate linkage. In various aspects, the 5′ cap is added to the RNA molecule via a chemical addition reaction. In exemplary embodiments, the RNA molecules are constructed based on chemical synthesis and/or enzymatic ligation reactions using procedures known in the art. See, for example, Sambrook et al., supra, and Ausubel et al., supra. In various aspects, the RNA molecules are produced outside of a cell via in vitro transcription techniques. In various aspects, the RNA molecules are synthetic RNA molecules produced by in vitro transcription.

In exemplary aspects, the liposome comprises less than or about 10 μg RNA molecules per 150 μg lipid mixture. In exemplary aspects, the liposome is made by incubating about 10 μg RNA with about 150 μg liposomes. In alternative aspects, the liposome comprises more RNA molecules per mass of lipid mixture. For example, the liposome may comprise more than 10 μg RNA molecules per 150 μg liposomes. The liposome in some instances comprises more than 15 μg RNA molecules per 150 μg liposomes or lipid mixture. In some aspects, the RNA molecule encodes a protein or is an antisense molecule. The protein is, in some aspects, selected from the group consisting of: a tumor antigen, a co-stimulatory molecule, a cytokine, a growth factor, a lymphokine, (including, e.g., cytokines and growth factors that are effective in inhibiting tumor metastasis, cytokines or growth factors that have been shown to have an antiproliferative effect on at least one cell population. Such cytokines, lymphokines, growth factors, or other hematopoietic factors include, but are not limited to: M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNFα, TNF1, TNF2, G-CSF, Meg-CSF, GM-CSF, thrombopoietin, stem cell factor, and erythropoietin. Additional growth factors for use herein include angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor α, cytokine-induced neutrophil chemotactic factor 1, cytokine-induced neutrophil, chemotactic factor 2 α, cytokine-induced neutrophil chemotactic factor 2 β, β endothelial cell growth factor, endothelin 1, epithelial-derived neutrophil attractant, glial cell line-derived neutrophic factor receptor a 1, glial cell line-derived neutrophic factor receptor a 2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, pre-B cell growth stimulating factor, stem cell factor, stem cell factor receptor, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, and chimeric proteins and biologically or immunologically active fragments thereof. In exemplary aspects, the tumor antigen is an antigen derived from a viral protein, an antigen derived from point mutations, or an antigen encoded by a cancer-germline gene. In exemplary aspects, the tumor antigen is pp65, p53, KRAS, NRAS, MAGEA, MAGEB, MAGEC, BAGE, GAGE, LAGE/NY-ESO1, SSX, tyrosinase, gp100/pme117, Melan-A/MART-1, gp75/TRP1, TRP2, CEA, RAGE-1, HER2/NEU, WT1. In exemplary aspects, the co-stimulatory molecule is selected from the group consisting of: CD80 and CD86.

In certain instances, the RNA molecule encodes or is an antisense molecule, optionally, an siRNA, shRNA, or miRNA. The antisense molecule can be one which mediates RNA interference (RNAi). As known by one of ordinary skill in the art, RNAi is a ubiquitous mechanism of gene regulation in plants and animals in which target mRNAs are degraded in a sequence-specific manner (Sharp, Genes Dev., 15, 485-490 (2001); Hutvagner et al., Curr. Opin. Genet. Dev., 12, 225-232 (2002); Fire et al., Nature, 391, 806-811 (1998); Zamore et al., Cell, 101, 25-33 (2000)). The natural RNA degradation process is initiated by the dsRNA-specific endonuclease Dicer, which promotes cleavage of long dsRNA precursors into double-stranded fragments between 21 and 25 nucleotides long, termed small interfering RNA (siRNA; also known as short interfering RNA) (Zamore, et al., Cell. 101, 25-33 (2000); Elbashir et al., Genes Dev., 15, 188-200 (2001); Hammond et al., Nature, 404, 293-296 (2000); Bernstein et al., Nature, 409, 363-366 (2001)). siRNAs are incorporated into a large protein complex that recognizes and cleaves target mRNAs (Nykanen et al., Cell, 107, 309-321 (2001). It has been reported that introduction of dsRNA into mammalian cells does not result in efficient Dicer-mediated generation of siRNA and therefore does not induce RNAi (Caplen et al., Gene 252, 95-105 (2000); Ui-Tei et al., FEBS Lett, 479, 79-82 (2000)). The requirement for Dicer in maturation of siRNAs in cells can be bypassed by introducing synthetic 21-nucleotide siRNA duplexes, which inhibit expression of transfected and endogenous genes in a variety of mammalian cells (Elbashir et al., Nature, 411: 494-498 (2001)).

In this regard, the RNA molecule in some aspects mediates RNAi and in some aspects is a siRNA molecule specific for inhibiting the expression of a protein. The term “siRNA” as used herein refers to an RNA (or RNA analog) comprising from about 10 to about 50 nucleotides (or nucleotide analogs) which is capable of directing or mediating RNAi. In exemplary embodiments, an siRNA molecule comprises about 15 to about 30 nucleotides (or nucleotide analogs) or about 20 to about 25 nucleotides (or nucleotide analogs), e.g., 21-23 nucleotides (or nucleotide analogs). The siRNA can be double or single stranded, preferably double-stranded.

In alternative aspects, the RNA molecule is alternatively a short hairpin RNA (shRNA) molecule specific for inhibiting the expression of a protein. The term “shRNA” as used herein refers to a molecule of about 20 or more base pairs in which a single-stranded RNA partially contains a palindromic base sequence and forms a double-strand structure therein (i.e., a hairpin structure). An shRNA can be an siRNA (or siRNA analog) which is folded into a hairpin structure. shRNAs typically comprise about 45 to about 60 nucleotides, including the approximately 21 nucleotide antisense and sense portions of the hairpin, optional overhangs on the non-loop side of about 2 to about 6 nucleotides long, and the loop portion that can be, e.g., about 3 to 10 nucleotides long. The shRNA can be chemically synthesized. Alternatively, the shRNA can be produced by linking sense and antisense strands of a DNA sequence in reverse directions and synthesizing RNA in vitro with T7 RNA polymerase using the DNA as a template.

Though not wishing to be bound by any theory or mechanism it is believed that after shRNA is introduced into a cell, the shRNA is degraded into a length of about 20 bases or more (e.g., representatively 21, 22, 23 bases), and causes RNAi, leading to an inhibitory effect. Thus, shRNA elicits RNAi and therefore can be used as an effective component of the disclosure. shRNA may preferably have a 3′-protruding end. The length of the double-stranded portion is not particularly limited, but is preferably about 10 or more nucleotides, and more preferably about 20 or more nucleotides. Here, the 3′-protruding end may be preferably DNA, more preferably DNA of at least 2 nucleotides in length, and even more preferably DNA of 2-4 nucleotides in length.

In exemplary aspects, the antisense molecule is a microRNA (miRNA). As used herein the term “microRNA” refers to a small (e.g., 15-22 nucleotides), non-coding RNA molecule which base pairs with mRNA molecules to silence gene expression via translational repression or target degradation. microRNA and the therapeutic potential thereof are described in the art. See, e.g., Mulligan, MicroRNA: Expression, Detection, and Therapeutic Strategies, Nova Science Publishers, Inc., Hauppauge, N.Y., 2011; Bader and Lammers, “The Therapeutic Potential of microRNAs” Innovations in Pharmaceutical Technology, pages 52-55 (March 2011).

In certain instances, the RNA molecule is an antisense molecule, optionally, an siRNA, shRNA, or miRNA, which targets a protein of an immune checkpoint pathway for reduced expression. In various aspects, the protein of the immune checkpoint pathway is CTLA-4, PD-1, PD-L1, PD-L2, B7-H3, B7-H4, TIGIT, LAG3, CD112 TIM3, BTLA, or co-stimulatory receptor: ICOS, OX40, 41BB, or GITR. The protein of the immune-checkpoint pathway in certain instances is CTLA4, PD-1, PD-L1, B7-H3, B7H4, or TIM3. Immune checkpoint signaling pathways are reviewed in Pardoll, Nature Rev Cancer 12(4): 252-264 (2012).

In exemplary instances, the liposome comprises a mixture of RNA molecules, e.g., RNA isolated from cells from a human In some aspects, the human has a tumor and the mixture of RNA is RNA isolated from the tumor of the human In exemplary aspects, the human has cancer, optionally, any cancer described herein. Optionally, the tumor from which RNA is isolated is selected from the group consisting of: a glioma, (including, but not limited to, a glioblastoma), a medulloblastoma, a diffuse intrinsic pontine glioma, or a peripheral tumor with metastatic infiltration into the central nervous system (e.g., melanoma or breast cancer). In exemplary aspects, the tumor from which RNA is isolated is a tumor of a cancer, e.g., any of these cancers described herein.

In exemplary aspects of the present disclosure, the liposome further comprises iron oxide nanoparticles (IONPs). Optionally, the IONPs are present in the core of the liposome. In exemplary aspects, the liposomes comprising IONPs are magnetic due to the inclusion of the IONPs. Accordingly, in some aspects, the liposomes comprising IONPs are called “magnetic liposomes” or “magnetoliposomes”. In certain instances, the IONPs is about 1% to about 20% of the total liposome mass, e.g., about 5% to about 20%, about 10% to about 20%, about 15% to about 20%, about 1% to about 15%, about 1% to about 10%, about 1% to about 5%. The IONPs in some aspects is about 5% to about 15% of the total liposome mass. In certain cases, the IONPs is about 12%±3% of the total liposome mass. Each IONP in the core has a diameter of about 10 nm to about 200 nm, in exemplary aspects. In some instance, each IONP has a diameter or about 20 nm about 30 nm, about 40 nm, about 50 nm, about 150 nm, about 160 nm, about 170 nm, about 180 nm, about 190 nm, about 200 nm, or about 60 nm to about 140 nm. (e.g., about 65 nm, about 70 nm, about 80 nm, about 90 nm, about 100 nm, about 110 nm, about 120 nm, about 130 nm, or about 140 nm). In exemplary aspects, each IONP has a diameter of about 130 nm±5 nm.

A cell comprising (e.g., transfected with) a liposome of the present disclosure is further provided herein. In exemplary aspects, the cell is any type of cell that can contain the presently disclosed liposome. The cell in some aspects is a eukaryotic cell, e.g., plant, animal, fungi, or algae. In alternative aspects, the cell is a prokaryotic cell, e.g., bacteria or protozoa. In exemplary aspects, the cell is a cultured cell. In alternative aspects, the cell is a primary cell, i.e., isolated directly from an organism, e.g., a human. The cell may be an adherent cell or a suspended cell, i.e., a cell that grows in suspension. The cell in exemplar aspects a mammalian cell. Most preferably, the cell is a human cell. The cell can be of any cell type, can originate from any type of tissue, and can be of any developmental stage. In exemplary aspects, the cell comprising the liposome is an antigen presenting cell (APC). As used herein, “antigen presenting cell” or “APC” refers to an immune cell that mediates the cellular immune response by processing and presenting antigens for recognition by certain T cells. In exemplary aspects, the APC is a dendritic cell, macrophage, Langerhans cell or a B cell. In exemplary aspects, the APC is a dendritic cell (DC). In exemplary aspects, when the cells are administered to a subject, e.g., a human, the cells are autologous to the subject. In exemplary instances, the immune cell is a tumor associated macrophage (TAM).

Also provided by the present disclosure is a population of cells wherein at least 50%, at least 60%, at least 70%, at least 80%, at least 90%, or at least 95% of the population are cells comprising (e.g., transfected with) a liposome of the present disclosure. The population of cells in some aspects is heterogeneous cell population or, alternatively, in some aspects, is a substantially homogeneous population, in which the population comprises mainly cells comprising a liposome of the present disclosure.

Provided herein are compositions comprising a liposome of the present disclosure, a cell comprising the liposome of the present disclosure, a population of cells of the present disclosure, or any combination thereof, and a pharmaceutically acceptable carrier, excipient or diluent. In exemplary aspects, the composition is a pharmaceutical composition intended for administration to a human. In exemplary aspects, the composition is a sterile composition. In exemplary instances, the composition comprises a plurality of liposomes of the present disclosure. Optionally, at least 50% of the liposomes of the plurality have a diameter between about 100 nm to about 250 nm and, optionally, have a core comprising IONPs.

In exemplary aspects, the composition of the present disclosure may comprise additional components other than the liposome, cell comprising the liposome, or population of cells. The composition, in various aspects, comprises any pharmaceutically acceptable ingredient, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, U K, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

The composition of the present disclosure can be suitable for administration by any acceptable route, including parenteral and subcutaneous. Other routes include intravenous, intradermal, intramuscular, intraperitoneal, intranodal and intrasplenic, for example. In exemplary aspects, when the composition comprises the liposomes (not cells comprising the liposomes), the composition is suitable for systemic (e.g., intravenous) administration. In exemplary aspects, when the composition comprises cells comprising the liposomes (and not liposomes outside of cells), the composition is suitable for intradermal administration.

If the composition is in a form intended for administration to a subject, it can be made to be isotonic with the intended site of administration. For example, if the solution is in a form intended for administration parenterally, it can be isotonic with blood. The composition typically is sterile. In certain embodiments, this may be accomplished by filtration through sterile filtration membranes. In certain embodiments, parenteral compositions generally are placed into a container having a sterile access port, for example, an intravenous solution bag, or vial having a stopper pierceable by a hypodermic injection needle, or a prefilled syringe. In certain embodiments, the composition may be stored either in a ready-to-use form or in a form (e.g., lyophilized) that is reconstituted or diluted prior to administration.

Methods of Manufacture

The present disclosure provides methods of making a liposome. In exemplary embodiments, the method comprises (A) mixing DOTAP and cholesterol at a DOTAP:cholesterol ratio of about 3:1 by mass to form a lipid mixture, (B) drying the lipid mixture, (C) rehydrating the lipid mixture with a rehydration solution to form a rehydrated lipid mixture, (D) incubating the rehydrated lipid mixture at a temperature greater than about 40° C. and intermittently vortexing the rehydrated lipid mixture to form liposomes. In exemplary aspects, the method further comprises incubating the liposomes for more than 12 hours after step (D), and, optionally, further comprises sonicating the liposomes. In exemplary aspects, the method further comprises incubating the liposomes for more than 12 hours at about 20° C. to about 30° C. or about 2° C. to about 4° C. after step (D). In exemplary instances, the method further comprises filtering the liposomes through a filter of at least 150 nm, optionally, wherein the liposomes are filtered through a 200 nm filter and/or a 450 nm filter. In some aspects, the liposomes are filtered through a 450 nm filter and a 200 nm filter, optionally, wherein the liposomes are sequentially filtered through a 450 nm filter followed by 200 nm filter. In exemplary instances, the method further comprises incubating the liposomes with RNA molecules. In some aspects, the method comprises mixing about 7.5 pg±0.75 DOTAP and about 2.5 μg±0.25 pg cholesterol to form the lipid mixture. In some aspects, the DOTAP and cholesterol are dissolved in chloroform to form the lipid mixture. In some aspects, nitrogen gas is used to dry the lipid mixture. The rehydration solution in exemplary aspects is a buffer, optionally, a phosphate buffered saline (PBS). In exemplary instances, the rehydrated lipid mixture is incubated in a water bath at a temperature of about 50° C. and vortexed about every 10 minutes to form liposomes. In exemplary aspects, the lipid mixture or the rehydration solution further comprises iron oxide nanoparticles (IONPs). Optionally, the rehydration solution comprises a solution of carboxylated iron oxide nanoparticles, wherein each carboxylated iron oxide nanoparticle has a diameter of about 50 nm to about 150 nm. In exemplary instances, the method further comprises adding IONPs the lipid mixture or the rehydration solution. In some aspects, each IONP has a diameter of about 10 nm to about 200 nm. Optionally, each IONP has a diameter of about 60 nm to about 140 nm or about 10 nm to about 30 nm. In some aspects, the lipid mixture or rehydration solution comprises at least about 1 μg IONPs per 10 mg lipid mixture, at least about 100 μg IONPs per 10 mg lipid mixture, at least about 1 mg IONPs per 10 mg lipid mixture, or at least about 1.5 mg IONPs per 10 mg lipid mixture, optionally, wherein the lipid mixture or rehydration solution comprises no more than about 5 mg IONPs per 10 mg lipid mixture. In some instances, the method comprises incubating the liposomes with RNA molecules. The RNA molecules in some aspects are any of those described herein, e.g., RNA molecules encoding a tumor antigen, a cytokine, an antisense molecule. In some instances, the RNA molecules are isolated for tumor cells obtained from a subject. In some aspects, about 5 μg RNA molecules is incubated with about every 75 μg lipids of the liposomes. In some aspects, the method comprises incubating the liposomes with RNA molecules at a RNA molecule:DOTAP ratio of about 1:15 by mass. In exemplary instances, about 10 μg RNA molecules is incubated with about every 150 μg liposomes when the liposomes comprise IONPs.

The liposomes made by any of the methods of making a liposome described herein are provided by the present disclosure. The liposomes may be used to transfect a cell. Accordingly, the cell comprising (e.g., transfected with) a liposome made by any of the methods of making a liposome described herein is provided by the present disclosure. In exemplary instances, the cell is any of the cells described herein, including, but not limited to an antigen presenting cell (APC). In exemplary instances, the cells is a dendritic cell (DC). In exemplary embodiments, the cell is part of a population of cells. Accordingly, populations of cells comprising a cell comprising (transfected with) a liposome made by any of the methods of making a liposome described herein are provided. In some aspects, at least 50% of the population of cells are cells comprising (e.g., transfected with) a liposome.

Also provided herein is composition comprising a liposome made by any of the methods of making a liposome described herein are provided by the present disclosure, a cell comprising the liposome, a population of cells comprising a cell comprising the liposome, or any combination thereof, and a pharmaceutically acceptable carrier, excipient or diluent. In exemplary aspects, the composition may be in accordance with the above-described compositions. In some aspects, the composition comprises a plurality of liposomes, wherein at least 50% of the liposomes have a diameter between about 100 nm to about 250 nm.

Methods of Use

As shown herein, compared to RNA electroporation, magnetic liposomes of the present disclosure comprising IONPs and RNA caused enhanced production of antiviral cytokines (IFN-alpha) and DC migration to lymph nodes. Such liposomes also produced a reduction in T2*-weighted MRI intensity and an increase in MRI-detected lymph node size that correlated directly with the number of iron oxide loaded cells in treated lymph nodes. These data suggest that magnetic liposomes of the present disclosure comprising IONPs and RNA enhance DC activation and allow noninvasive cell tracking with MRI. Without being bound to any particular theory, these data support the use of magnetic liposomes of the present disclosure comprising IONPs and RNA for predicting antitumor immune responses and for using MRI-detected DC migration as a biomarker for vaccine efficacy.

Provided herein are methods of delivery RNA to cells of a tumor, e.g., a brain tumor, comprising intravenously administering a presently disclosed composition, wherein the composition comprises the liposome. Also provided herein are methods of delivering RNA to cells in a microenvironment of a tumor, optionally a brain tumor. In exemplary embodiments, the method comprises intravenously administering a presently disclosed composition, wherein the composition comprises the liposome. In some aspects, the liposome comprises an siRNA targeting a protein of a immune checkpoint pathway, optionally, PDL1. In various aspects, the cells in the microenvironment are antigen-presenting cells (APCs), optionally, tumor associated macrophages. The present disclosure also provides methods of activating antigen-presenting cells in a brain tumor microenvironment. In exemplary embodiments, the method comprises intravenously administering a presently disclosed composition, wherein the composition comprises the liposome.

The present disclosure provides methods of delivering RNA molecules to cells. In exemplary embodiments, the method comprises incubating the cells with the liposomes comprising ribonucleic acid (RNA) molecules and a lipid mixture comprising DOTAP and cholesterol, wherein the DOTAP and cholesterol are present in the lipid mixture at a DOTAP:cholesterol ratio of about 3:1 by mass. In exemplary aspects, the liposomes comprise IONPs as described herein. In exemplary instances, the cells are incubated with the liposomes in the presence of a magnetic field. In exemplary aspects, the magnetic field is a static magnetic field. In exemplary aspects, the magnetic field is an oscillating magnetic field. In exemplary aspects, the magnetic field is a magnetic field having a strength of about 100 mT. In exemplary instances, the cells are incubated with the liposomes in the presence of a magnetic field for time of less than about 2 hours or less than about 1 hour, optionally, wherein the cells are incubated with the liposomes in the presence of a magnetic field for about 30 minutes±10 minutes. In exemplary instances, the cells are antigen-presenting cells (APCs), optionally, dendritic cells (DCs). In various instances, the APCs (e.g., DCs) are obtained from a subject. In certain aspects, the RNA molecules are isolated from tumor cells obtained from a subject, e.g., a human In certain aspects, the RNA molecules are antisense molecules that target a protein of interest for reduced expression. In exemplary aspects, the RNA molecules are siRNA molecules that target a protein of the immune checkpoint pathway. Suitable proteins of the immune checkpoint pathway are known in the art and also described herein. In various instances, the siRNA target PDL1.

Once RNA has been delivered to the cells, the cells may be administered to a subject for treatment of a disease. Accordingly, the present disclosure provides a method of treating a subject with a disease. In exemplary embodiments, the method comprises delivering RNA molecules to cells of the subject in accordance with the above-described method of delivering RNA molecules to cells. In some aspects, RNA molecules are delivered to the cells ex vivo and the cells are administered to the subject. Alternatively, the method comprises administering the liposomes directly to the subject. In exemplary embodiments, the method of treating a subject with a disease comprises administering a composition of the present disclosure in an amount effective to treat the disease in the subject. In exemplary aspects, the disease is cancer, and, in some aspects, the cancer is located across the blood brain barrier and/or the subject has a tumor located in the brain. In some aspects, the tumor is a glioma, a low grade glioma or a high grade glioma, specifically a grade III astrocytoma or a glioblastoma. Alternatively, the tumor could be a medulloblastoma or a diffuse intrinsic pontine glioma. In another example, the tumor could be a metastatic infiltration from a non-CNS tumor e.g. breast cancer, melanoma, or lung cancer. In exemplary aspects, the composition comprises the liposomes, and optionally, the composition comprising the liposomes are intravenously administered to the subject. In alternative aspects, the composition comprises cells transfected with the liposome. Optionally, the cells of the composition are APCs, optionally, DCs. In exemplary aspects, the composition comprising the cells comprising the liposome is intradermally administered to the subject, optionally, wherein the composition is intradermally administered to the groin of the subject. In exemplary instances, the DCs are isolated from white blood cells (WBCs) obtained from the subject, optionally, wherein the WBCs are obtained via leukapheresis. In some aspects, the RNA molecules encode a tumor antigen. In some aspects, the RNA molecules are isolated from tumor cells, e.g., tumor cells are cells of a tumor of the subject. The liposomes in exemplary aspects, comprise IONPs and the method further comprises tracking migration of the cells comprising the liposomes within the subject. The tracking in exemplary aspects comprises magnetic resonance imaging (MRI). For example, the tracking comprises conducting MRI on one or more lymph nodes of the subject, optionally, MRI is conducted on the lymph nodes before and after administration of the composition or the cells. In exemplary aspects, the methods of treating comprises comparing the T2*-weighted MRI intensity of the lymph node comprising DCs transfected with liposomes comprising IONPs to the T2*-weighted MRI intensity of a control, untreated lymph node. The method optionally comprises measuring lymph node size of the subject via MRI. Also, optionally, the method comprises comparing the lymph node size of the lymph node comprising DCs transfected with liposomes comprising IONPs lymph node compared to the lymph node size of the a control, untreated lymph node.

The terms “treat”, “treating” and “treatment” refer to eliminating, reducing, suppressing or ameliorating, either temporarily or permanently, either partially or completely, a clinical symptom, manifestation or progression of an event, disease or condition associated with an inflammatory disorder described herein. As is recognized in the pertinent field, drugs employed as therapeutic agents may reduce the severity of a given disease state, but need not abolish every manifestation of the disease to be regarded as useful therapeutic agents. Similarly, a prophylactically administered treatment need not be completely effective in preventing the onset of a condition in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient. One embodiment of the invention is directed to a method for determining the efficacy of treatment comprising administering to a patient therapeutic agent in an amount and for a time sufficient to induce a sustained improvement over baseline of an indicator that reflects the severity of the particular disorder.

As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating a disease of the present disclosure can provide any amount or any level of treatment. Furthermore, the treatment provided by the method may include treatment of one or more conditions or symptoms or signs of the disease being treated. For instance, the treatment method of the presently disclosure may inhibit one or more symptoms of the disease. Also, the treatment provided by the methods of the present disclosure may encompass slowing the progression of the disease. The term “treat” also encompasses prophylactic treatment of the disease. Accordingly, the treatment provided by the presently disclosed method may delay the onset or reoccurrence/relapse of the disease being prophylactically treated. In exemplary aspects, the method delays the onset of the disease by 1 day, 2 days, 4 days, 6 days, 8 days, 10 days, 15 days, 30 days, two months, 4 months, 6 months, 1 year, 2 years, 4 years, or more. The prophylactic treatment encompasses reducing the risk of the disease being treated. In exemplary aspects, the method reduces the risk of the disease 2-fold, 5-fold, 10-fold, 20-fold, 50-fold, 100-fold, or more.

With regard to the foregoing methods, the liposomes or the composition comprising the same in some aspects is systemically administered to the subject. Optionally, the method comprises administration of the liposomes or composition by way of parenteral administration. In various instances, the liposome or composition is administered to the subject intravenously.

In various aspects, the liposome or composition is administered according to any regimen including, for example, daily (1 time per day, 2 times per day, 3 times per day, 4 times per day, 5 times per day, 6 times per day), three times a week, twice a week, every two days, every three days, every four days, every five days, every six days, weekly, bi-weekly, every three weeks, monthly, or bi-monthly. In various aspects, the liposomes or composition is/are administered to the subject once a week.

The term “therapeutically effective amount” refers to an amount of therapeutic agent that is effective to ameliorate or lessen symptoms or signs of disease associated with a disease or disorder.

In exemplary aspects, treatment of the disease is achieved by enhancing in a subject an immune response against the disease. Accordingly, the present disclosure provides methods of enhancing in a subject an immune response against a disease, e.g., a tumor or cancer. In exemplary embodiments, the method comprises administering to the subject a liposome of the present disclosure in an amount effective to enhance the immune response in the subject against the disease, e.g., tumor or cancer. In exemplary aspects, the enhanced immune response is evident by increased activation of dendritic cells which is demonstrated by, e.g., enhanced expression of genes related to DC activation, enhanced expression of co-stimulatory molecules on the surface of DCs, increased production of anti-viral cytokines (e.g., IFNα), increased T cell stimulation (as shown by e.g., increased IFN-γ production by T-cells upon contact with the activated DCs), increased migration to lymph nodes, and enhanced inhibition of tumor growth. Accordingly, the present disclosure further provides methods of increasing activation of DCs or activating DCs. In exemplary embodiments, the method comprises administering to the subject a liposome of the present disclosure in an amount effective to increase activation of DCs or in an amount effective to activate DCs. The present disclosure also accordingly provides methods of increasing production of anti-viral cytokines (e.g., IFNα). In exemplary embodiments, the method comprises administering to the subject a liposome of the present disclosure in an amount effective to increase production of the anti-viral cytokine in the subject. Further provided are methods of increasing T cell stimulation in a subject. In exemplary embodiments, the method comprises administering to the subject a liposome of the present disclosure in an amount effective to increase T cell stimulation in the subject. In various aspects, the increase in T-cell stimulation is evident from an increase in T-cell production of IFN-γ. The present disclosure further provides methods of increasing T cell production of IFN-γ. In exemplary embodiments, the method comprises contacting T cells with a presently disclosed dendritic cell (DC), optionally, wherein the liposome comprise IONPs and the DC is transfected with the liposome in the presence of a magnetic field. A method of increasing dendritic cell (DC) migration to a lymph node in a subject is further provided. In exemplary embodiments, the method comprises administering to the subject a presently disclosed composition in an amount effective to increase DC migration to the lymph node. As used herein, the term “increase” and “enhance” and words stemming therefrom may not be a 100% or complete increase or enhancement. Rather, there are varying degrees of increasing or enhancing of which one of ordinary skill in the art recognizes as having a potential benefit or therapeutic effect. In exemplary embodiments, the increase or enhancement provided by the methods is at least or about a 10% increase or enhancement (e.g., at least or about a 20% increase or enhancement, at least or about a 30% increase or enhancement, at least or about a 40% increase or enhancement, at least or about a 50% increase or enhancement, at least or about a 60% increase or enhancement, at least or about a 70% increase or enhancement, at least or about a 80% increase or enhancement, at least or about a 90% increase or enhancement, at least or about a 95% increase or enhancement, at least or about a 98% increase or enhancement).

The present disclosure further provides methods of increasing dendritic cell (DC) migration to a lymph node in a subject. In exemplary embodiments, the method comprises administering to the subject a presently disclosed composition, in an amount effective to increase DC migration to the lymph node. The present disclosure further provides methods of activating dendritic cells (DCs) in a subject. In exemplary embodiments, the method comprises administering to the subject a presently disclosed composition, in an amount effective to activate DCs in the subject. In various instances, the DCs lead to superior inhibition of tumor growth. Thus, the present disclosure provides methods of inhibiting tumor growth in a subject. In exemplary embodiments, the method comprises administering to the subject a liposome of the present disclosure in an amount effective to inhibit tumor growth in the subject. In various aspects, the method comprises administering to the subject a presently disclosed composition, in an amount effective to activate DCs in the subject. In various aspects of these methods, the method comprises incubating DCs obtained from a subject with liposomes of the present disclosure and administering the DCs comprising the liposomes into the subject. The method in various instances comprises tracking migration of the DCs comprising the liposomes to lymph nodes in the subject using MRI. The tracking of the DCs in various embodiments may be used to predict a subject's response to therapy with the DCs comprising the liposomes. As used herein, the term “inhibit” and words stemming therefrom may not be a 100% or complete inhibition or abrogation. Rather, there are varying degrees of inhibition of which one of ordinary skill in the art recognizes as having a potential benefit or therapeutic effect. In exemplary embodiments, the inhibition provided by the methods is at least or about a 10% inhibition (e.g., at least or about a 20% inhibition, at least or about a 30% inhibition, at least or about a 40% inhibition, at least or about a 50% inhibition, at least or about a 60% inhibition, at least or about a 70% inhibition, at least or about a 80% inhibition, at least or about a 90% inhibition, at least or about a 95% inhibition, at least or about a 98% inhibition).

A method of tracking dendritic cell (DC) migration to a lymph node in a subject is provided by the present disclosure. In exemplary embodiments, the method comprises (i) treating the subject in accordance with the presently disclosed method of treating a subject with a disease, as described herein, wherein the cells are DCs and the liposomes comprise IONPs, and (ii) performing magnetic resonance imaging (MRI) on one or more lymph nodes of the subject. In exemplary aspects, the method comprises determining the T2*-weighted MRI intensity of one or more lymph nodes, wherein lymph nodes exhibiting a reduction in T2*-weighted MRI intensity, relative to the T2*-weighted MRI intensity of a control, untreated lymph node, represent lymph nodes to which DCs migrated. In certain aspects, one or more lymph nodes are the inguinal lymph nodes of the subject, optionally, wherein the composition is intradermally administered to the groin of the subject. In exemplary instances, MRI is conducted on the lymph nodes before and after administration of the composition or the cells, optionally, wherein MRI is conducted before and about 48 hours after administration and, optionally, about 72 hours after administration. In some aspects, the method comprises comparing the T2*-weighted MRI intensity of the lymph node comprising DCs transfected with liposomes comprising IONPs to the T2*-weighted MRI intensity of a control, untreated lymph node. In exemplary aspects, the method comprises measuring lymph node size of the subject via MRI, and optionally further comprises comparing the lymph node size of the lymph node comprising DCs transfected with liposomes comprising IONPs lymph node compared to the lymph node size of the a control, untreated lymph node

A method of determining a subject's therapeutic response to dendritic cell (DC) vaccination therapy in a subject is also provided by the present disclosure. In exemplary embodiments, the method comprises (i) treating the subject in accordance with the presently disclosed method of treating, wherein the cells are DCs and the liposomes comprise IONPs, and (ii) tracking DC migration to a lymph node in accordance with any of the presently disclosed methods of tracking DC migration. In some aspects, when T2*-weighted MRI intensity of treated lymph nodes is reduced, the DC vaccination therapy is determined to lead to a positive therapeutic response in the subject. In exemplary instances, the positive therapeutic response comprises prolonged progression free and overall survival of the subject for at least 4 weeks post-administration of therapy. In some aspects, the positive therapeutic response comprises prolonged progression free and overall survival of the subject for at least 8 to 12 weeks post-administration of therapy.

The present disclosure also provides a method of monitoring therapeutic response to dendritic cell (DC) vaccination therapy in a subject. In exemplary embodiments, the method comprises tracking DC migration to a lymph node in accordance with any one of the presently disclosed methods of tracking at a first time point and at a second time point, wherein, when T2*-weighted MRI intensity of treated lymph nodes is reduced at the second time point relative to the T2*-weighted MRI intensity of the treated lymph nodes at the first time point, the therapeutic response to DC vaccination therapy is effective.

Also provided is a method of increasing T cell production of IFN-γ, comprising contacting T cells with a presently disclosed dendritic cell (DC) comprising or transfected with a presently disclosed liposome, optionally, wherein the liposome comprise IONPs and the DC is transfected with the liposome in the presence of a magnetic field.

Cancer

The cancer treatable by the methods disclosed herein may be any cancer, e.g., any malignant growth or tumor caused by abnormal and uncontrolled cell division that may spread to other parts of the body through the lymphatic system or the blood stream. In some embodiments, the cancer is a cancer in which an integrin and a G protein a subunit are expressed on the surface of the cells.

The cancer in some aspects is one selected from the group consisting of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. In particular aspects, the cancer is selected from the group consisting of: head and neck, ovarian, cervical, bladder and oesophageal cancers, pancreatic, gastrointestinal cancer, gastric, breast, endometrial and colorectal cancers, hepatocellular carcinoma, glioblastoma, bladder, lung cancer, e.g., non-small cell lung cancer (NSCLC), bronchioloalveolar carcinoma.

Subjects

In exemplary aspects, the subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human In some aspects, the human is an adult aged 18 years or older. In some aspects, the human is a child aged 17 years or less.

The invention, thus generally described, will be understood more readily by reference to the following example, which is provided by way of illustration and is not intended to limit the invention.

### EXAMPLES

### Example 1

This example demonstrates an exemplary method of making cholesterol-containing liposomes of the present disclosure and the use thereof for RNA delivery to dendritic cells (DCs).

A series of experiments were carried out to make cholesterol-containing liposomes that are highly efficient for RNA delivery to DCs. Control liposomes without any cholesterol were made as essentially described in Sayour et al., Oncoimmunology 6(1): e1256527 (2017).

Liposomes were made with varying amounts of cholesterol using a variation of the thin film rehydration technique. Each formulation had a total of 10 mg lipid. A summary of the formulations made are shown in the table below.

| | | | DOTAP: | |
|---|---|---|---|---|
| Formulation | DOTAP | Cholesterol | Cholesterol | % |
| # | (mg) | (mg) | Ratio (by mass) | cholesterol |
| 1 | 7.5 | 2.5 | 3:1 | 25 |
| 2 | 8.75 | 1.25 | 6:1 | 12.5 |
| 3 | 6.25 | 3.75 | 2:1 | 37 |
| 4 | 10 | 0 | na | 0 |

Briefly, for each formulation, DOTAP and cholesterol (or DOTAP alone) were dissolved in chloroform and added to a borosilicate glass tube. Chloroform was evaporated in nitrogen gas (N₂) and then without nitrogen before rehydration with PBS. Particles were then rehydrated with 2 mL PBS and incubated in a water bath at 50° C. and vortexed every 10 minutes for 1 hour to allow liposome formation. Liposomes were then left overnight at room temperature. The next day, liposomes were sonicated for 5 minutes at room temperature and filtered through a 450 nm filter followed by a 200 nm filter.

The transfection efficiency of liposomes containing cholesterol were compared to that of liposomes lacking cholesterol using an immortalized cell line of dendritic cells called DC2.4s with flow cytometry. Green fluorescence protein (GFP)-encoding RNA (GFP RNA) was loaded onto each type of liposome by incubating about 75 μg liposomes with about 5 μg RNA in buffer. The mixture was kept at room temperature for 15 to 20 min to allow RNA-loaded DOTAP liposomes to form. For cell transfection, about 27 μg RNA-loaded DOTAP liposomes were incubated with about 200,000 DC2.4 cells overnight. The percentage of GFP-positive (GFP⁺) cells was assessed via flow cytometry. Briefly, transfected cells were washed with PBS before addition of about 300 μL of 0.05% trypsin. After 5 minutes, serum containing media was used to neutralize the trypsin and cells were transferred to fluorescence activated cell sorting (FACS) tubes. These cells were washed with PBS and resuspended in FACS buffer for flow cytometric analysis of GFP expression on a FACS Calibur.

As shown in FIG. 1A, the addition of cholesterol to DOTAP liposomes greatly enhanced the level of cell transection. Liposomes comprising 12-37% cholesterol achieved at least a 3-fold increase in GFP⁺ cells, as determined by flow cytometry. While the highest level of cell transfection was achieved with liposomes formulated with 25% cholesterol content, the level of cell transfection using liposomes formulated with 37% cholesterol content also was significantly greater than liposomes lacking any cholesterol. Liposomes containing 25% cholesterol content were used in subsequent studies.

In another experiment, GFP RNA was labeled with Cy5 via an Arcturus Turbo labeling kit and incorporated into DOTAP liposomes with either 0% or 25% cholesterol by mass made as essentially described above. Liposomes were incubated with DC2.4 dendritic cells overnight and analyzed via flow cytometry. Liposomes containing cholesterol (Chol-RNA-NP) delivered RNA to a significantly higher proportion of cells than standard RNA-liposomes without reducing viability (FIGS. 1B and 1C).

The transfection efficiency of liposomes containing cholesterol were compared to that of liposomes lacking cholesterol using primary bone marrow-derived DCs (BMDCs) loaded with RNA isolated from a murine glioma cell line. Briefly, total tumor-derived RNA from tumor cells (KR158b-luciferase) was isolated using commercially available RNeasy mini kits (Qiagen) based on manufacturer instructions. Liposomes comprising either 0% or 25% cholesterol made as essentially described above were loaded with the RNA from KR158B-Luciferase tumor cells. These cells were then incubated for two days with KR158b-luciferase-specific T cells from other vaccinated animals. After 48 hours, supernatants were collected to evaluate IFN-gamma production, as a hallmark of T cell activation. IFN-gamma production was determined via an IFN-gamma ELISA (Invitrogen, BMS606) according to manufacturer instructions.

As shown in FIG. 1D, cholesterol-bearing liposomes elicited more IFN-gamma production from T cells, indicating that cholesterol-loaded liposomes increased the primary DC function of activating T cell responses.

These results demonstrate that liposomes comprising cholesterol deliver RNA to cells at a higher efficiency relative to liposomes without cholesterol. The data show that a 3:1 ratio by mass of DOTAP to cholesterol provides superior transfection efficiency for liposomes that can very efficiently transfect DCs which transfected DCs then activate T cells to produce IFN gamma. The results presented here suggest the clinical utility of the cholesterol-containing liposomes as part of a DC vaccination strategy for activating T cells.

### Example 2

This example demonstrates that cholesterol-containing liposomes can target immune cells in brain tumors.

Liposomes with 0% cholesterol or with 25% cholesterol (made with DOTAP at a 3:1 DOTAP:cholesterol ratio) were made as described in Example 1. The liposomes were loaded with Cy3- or Cy5-labeled RNA encoding ovalbumin (OVA RNA) and injected intravenously into mice with KR158b-luciferase tumors. KR158b-luciferase is a temozolomide and radiation resistant murine glioma line that recapitulates the hallmark findings of human glioblastoma including infiltration into surrounding brain tissue. After 24 hours, brains were extracted from 3 mice and preserved for immunofluorescence imaging. FIG. 2A represents an immunofluorescence image of the cortex and FIG. 2B represents an immunofluorescence image of the tumor. Tumors were extracted from a separate set of mice and the number of Cy5-labelled cells in each tumor was analyzed via flow cytometry. Untreated mice and mice treated with Cy5-labelled RNA alone served as negative controls. As shown in FIG. 2C, the number of Cy5+ cells in the brain tumor was highest for liposomes containing cholesterol.

A different brain tumor model was tested. RNA-liposomes with or without 25% cholesterol (made with DOTAP at a 3:1 DOTAP:cholesterol ratio) were loaded with Cy5-labelled RNA and injected intravenously into mice with GL261 tumors. GL261 is a murine gliosarcoma that is commonly used as a treatment model to represent human glioblastoma. After 24 hours, tumors were harvested to evaluate the number of Cy5-labelled cells with flow cytometry. As was the case for mice bearing KR158b-luciferase tumors, only the cholesterol-bearing liposomes delivered Cy3 or Cy5-labelled mRNA to cells in GL261 tumors (FIG. 2D). These results were significant, as they suggested that these canonically immune suppressive cells in the area immediately surrounding the brain tumor could be manipulated, thereby providing an unprecedented advantage for vaccination strategies using these liposomes.

DOTAP liposomes with varying amounts of cholesterol (0%, 12.5%, 25%, or 37%) were made as described above and loaded with Cy3-labelled OVA RNA. The RNA-loaded liposomes were then injected intravenously into C57B16 mice with KR158b-Luciferase tumors. Tumors from the mice were harvested after 24 hours and evaluated with flow cytometry. As shown in FIG. 3, there was a clear dependence on the amount of cholesterol in the liposome. Liposomes loaded with 25% cholesterol demonstrated the highest amount of Cy3+ cells in the tumor.

Further studies were carried out to determine the propensity of the liposomes to deliver RNA to certain cell types. Brain tumor slices from the mice in FIG. 2C were stained with an immunofluorescence antibody for the endothelial cell marker CD31. Images were then taken with a fluorescent microscope to evaluate the relationship between the delivered RNA and the endothelial vessels. As shown in FIG. 4, the RNA-loaded liposomes containing cholesterol did not co-localize exactly with cells expressing CD31. These results suggest that RNA is delivered to cells surrounding blood vessels but that not the endothelial cells themselves.

Further experiments were carried out to characterize the cells to which the liposomes were targeting upon injection. C57B16 mice received intracranial injections of 10,000 KR158b-Luciferase cells. After three weeks, mice received intravenous injection of liposomes composed of 25% Cholesterol and 75% DOTAP and loaded with Cy5-labeled RNA. Tumors were excised the next day, dissociated with papain, strained into a single cell suspension, and stained for flow cytometry with a Live/Dead Dye (LIVE/DEAD Fixable Near-IR Dead Cell Stain Kit, Invitrogen L10119) and antibodies for CD45 (PerCP-Cy5.5, BioLegend) and MHCII (FITC, ThermoFisher) in FACS buffer. Cells were then fixed in 4% paraformaldehyde (PFA) and evaluated with a BD LSR II Flow Cytometer. As shown in FIGS. 5A-5C, cholesterol containing RNA-loaded liposomes were found almost exclusively in CD45+ cells in the brain tumor. Additionally, about half of these cells were positive for MHC Class II. This experiment was repeated with GL61 tumors with nearly identical results. Since CD45 is a universal marker of bone marrow derived cells and MHC II is an integral part of the antigen presentation machinery only found on antigen presenting cells, this evidence indicates that cholesterol bearing RNA-liposomes efficiently deliver RNA to immune cells in brain tumors, of which about half are MHCII+ antigen presenting cells. RNA delivery to either CD45+ population is of interest from a therapeutic perspective because the CD45 compartment is known to be co-opted to support tumor growth inhibit antitumor immune responses. The MHCII+ antigen presenting cells in the tumor are thought to induce antigen specific immune tolerance, while the MHCII− cells include myeloid derived suppressor cells which are known to produce cytokines and express co-regulatory molecules that further inhibit antitumor immune responses.

In order to evaluate whether this enhanced RNA delivery was specific to brain tumors, studies were carried out to see if cholesterol containing RNA-loaded liposomes also enhanced RNA delivery to peripheral organs. C57B16 mice received intravenous injection of Cy5-labelled RNA-liposomes with 25% cholesterol by mass. Spleens, livers, and lungs were excised at 24 hours, strained into a single cell suspension, lysed, and stained with CD45 (PerCP-Cy5.5) and MHCII (FITC) for flow cytometry. As shown in FIGS. 6A-6C, cholesterol containing RNA-loaded liposomes were found in CD45+ cells in the liver to a greater extent than RNA-loaded liposomes containing 0% cholesterol. In the lungs and spleens, cholesterol containing RNA-loaded liposomes were found to about the same extent as RNA-loaded liposomes containing 0% cholesterol. RNA uptake in each of these peripheral organs was plotted against uptake in brain tumors (6D-F). In this analysis, a direct relationship between uptake in one organ with uptake in brain tumors would suggest a similar or related mechanism for the uptake in both sites. In each case, there was no significant relationship between uptake in that organ and uptake in brain tumors. These data suggest that uptake of cholesterol-bearing RNA-liposomes in brain tumors occurs through a mechanism distinct from the mechanism by which cholesterol-bearing RNA-liposomes are taken up in lung, spleen, or liver. These data also suggest that cholesterol-bearing RNA-liposomes are attractive candidates as vaccines to treat tumors of the lung and liver.

### Example 3

This example demonstrates a process of making a magnetic liposome of the present disclosure.

Several experiments aimed at making iron oxide (10)-loaded DOTAP liposomes useful for RNA delivery to dendritic cells (DCs) and useful as a magnetic resonance imaging (MRI) contrast imaging agent were designed and carried out.

The protocol for making control liposomes described in Example 1 was modified to include iron oxide. In a first series of experiments, starting materials in chloroform were sonicated, rehydrated with a rehydration material, followed by sonication. Table 1 details the different materials and conditions tested for making IO-loaded DOTAP liposomes. IO was made in-house and coated with oleic acid. For Expmt #1-5, DOTAP was present at a concentration of at least 350 μg and the starting materials included one or more of IO in chloroform, polyethylene glycol (PEG), N-methyl-2-pyrrolidone (NMP), and oleic acid. For Exmpt #6-7, 45 μg DOTAP and 10 μL of a 30 mg/mL IO solution were the starting materials. In Expmt #8 and 9, DOTAP was the sole starting material, and for Expmt 10, DOTAP was absent altogether. For Expmt #1-7, iron oxide was a starting material. In Expmt #8-10, iron oxide was used as a rehydration material.

TABLE 1

| | | | | | | | Treatment |
|---|---|---|---|---|---|---|---|
| Expmt | Starting Materials | | Sonication in | | Rehydration | Rehydration | after |
| # | (in chloroform) | | chloroform | | material | sonication | sonication |
| 1 | Starting Materials | | 1 | hr at RT | 1 mL distilled | 20 min | Magnetic |
| | (in chloroform) | | | | water | at 60° C. | Separation |
| 2 | 350 | μg DOTAP | 1 | hr at RT | 1 mL distilled | 20 min | Magnetic |
| | 9000 | μg oleic acid | | | water | at 60° C. | Separation |
| | 420 | μg PEG | | | | | |
| | 300 | ug IO in | | | | | |
| | chloroform | | | | | | |
| 3 | 475 | μg DOTAP | 1 | hr at RT | 1 mL distilled | 20 min | Freeze at −20 |
| | 300 | ug IO in | | | water | at 60° C. | and thaw on |
| | chloroform | | | | | | magnetic |
| | 10 | μl oleic acid | | | | | separator |
| 4 | 350 | μg DOTAP | 7 | hrs at RT | 1 mL distilled | 1 hr | Magnetic |
| | 9000 | μg oleic acid | | | water | | Separation |
| | 420 | μg PEG | | | | | |
| | 300 | ug IO in | | | | | |
| | chloroform | | | | | | |
| 5 | 2975 | μg DOTAP | 7 | hrs at RT | 1 mL distilled | 1 hr | Magnetic |
| | 9000 | μg oleic acid | | | water | | Separation |
| | 50 | μL NMP | | | | | |
| | 300 | ug IO in | | | | | |
| | chloroform | | | | | | |
| 6 | 2975 | μg DOTAP | 0.5 | hour | 1 mL distilled | 20 min | |
| | 1920 | μg PEG | | | water | at 60° C. | |
| | 9000 | μg oleic acid | | | | | |
| | 50 | μL NMP | | | | | |
| | 300 | ug IO in | | | | | |
| | chloroform | | | | | | |
| 7 | 20 | μL DOTAP | 0.5 | hour | 1 mL distilled | 20 min | |

| | | | | | |
|---|---|---|---|---|---|
| | 300 | ug IO | water | at 60° C. | |
| 8 | 2500 | μg DOTAP | 1 mL iron salt | 1 hr at 50° C. | Next day: |
| | | | solution 4.5 mL | | 4.5 mL |
| | | | | | NH3•H2O |
| | | | | | Shake at |
| | | | | | 60° C. |
| | | | | | for 1 hr |
| 9 | 2500 | μg DOTAP | 1 mL iron salt | 1 hr | Next day: |
| | | | solution 4.5 mL | at 50° C. | 4.5 mL |
| | | | | | NH3•H2O |
| | | | | | Sonication at |
| | | | | | 60° C. for 1 hr |

| | | | | |
|---|---|---|---|---|
| 10 | None | 1 mL iron salt | 1 hr | Next day: |
| | | solution 4.5 mL | at 50° C. | 4.5 mL |
| | | | | NH3•H2O |
| | | | | Shake at 60° C. |
| | | | | for 1 hr |

RT = room temperature; IO = iron oxide; NMP=

The resulting particles were analyzed via transmission electron microscopy (TEM). For Expmt #1-5, the TEM images showed that the iron oxide was in the oleic acid and not in the liposome. For Expmt #6, iron oxide was present outside of the liposome. For Expmt #7, many liposomes had very little to no IO. For Expmt #8-10, no liposomes with IO were made.

In a second series of experiments, DOTAP and IO made from different sources were used. In some experiments, oleic acid was a starting material. Table 2 details the different materials tested for making IO-loaded DOTAP liposomes. The conditions for each of these experiments were the same as Expmt #2. The resulting particles were analyzed via TEM.

TABLE 2

| | Starting Materials | |
|---|---|---|
| Expmt # | (in chloroform) | |
| 11 | 475 | μg DOTAP |
| | 9000 | μg oleic acid |
| | 300 | ug IO (Batch J) |
| 12 | 475 | μg DOTAP |
| | 9000 | μg oleic acid |
| | 3 | mg IO (Batch A) |
| 13 | 475 | μg DOTAP |
| | 9000 | μg oleic acid |
| | 300 | ug IO (Batch A) |
| 14 | 475 | μg DOTAP |
| | 20 | μL oleic acid |
| | 6 | mg IO (Batch A) |
| 15 | 475 | μg DOTAP |
| | 300 | ug IO (Batch A) |
| 16 | 2500 | μg DOTAP |
| | 600 | ug IO (Batch J) |

In each of Expmt #11-15, no liposomes were detected by TEM. For Expmt #16, liposomes and iron oxide particles were detected as separate entities.

In a third set of experiments, starting materials were sonicated, rehydrated with purified distilled water, and incubated at 50° C. with sonication every 10 minutes for 1 hour. Particles were then left overnight before a 5-minute sonication followed by filtration through 450 nm Whatman and 200 nm PALL filters. IO was added as a starting material or as a rehydration material or after filtration. Table 3 details the different materials tested for making IO-loaded DOTAP liposomes. The resulting particles were analyzed via transmission electron microscopy (TEM).

TABLE 3

| | Starting materials | |
|---|---|---|
| Expmt # | in chloroform | Rehydration material |
| 17 | 10 mg DOTAP | 4 mL PBS |
| | 10 nM oleic acid | |
| | coated IO | |
| | nanoparticles | |
| 18 | 10 mg DOTAP | 4 mL PBS with 400 μg of |
| | | 10 nm PEGylated IO |
| | | nanoparticles (Sigma) |
| 19 | 10 mg DOTAP | 4 mL PBS with 200 μg of |
| | | 10 nm PEGylated IO |
| | | nanoparticles (Sigma) |
| 20 | 10 mg DOTAP | 2 mL PBS later filtered |
| | | into 400 μg of 10 nm |
| | | PEGylated IO nanoparticles |
| | | (Sigma) in 2 mL |
| | | PBS |

For Expmt #17-20, 5 μg RNA was added to a sample of 75 μg of each liposome formulation. These RNA-loaded liposomes were then added to an electrophoresis gel and run under a voltage of 80 mV to evaluate the amount of unbound RNA. These RNA-loaded liposomes were then evaluated with Cryo TEM to determine whether iron oxide was bound within liposomes. Unbound RNA was identified in liposomes from Expmt #20. For Expmt #17-19, each particle construct bound to RNA, but no IO was detected in the liposomes.

In a fourth set of experiments, varying amounts of PEGylated iron oxide nanoparticles (10 nm in diameter; 200 μg, 400 μg, 1 mg, or 1.5 mg) or 400 μg ferraheme (FH) was added to a lipid cake. FH is a contrast agent consisting of 30 nm dextran coated IO nanoparticles. Table 4 details the materials used.

TABLE 4

| Expmt | | | | | IO | | | |
|---|---|---|---|---|---|---|---|---|
| # | DOTAP | | Cholesterol | | nanoparticles | | FH | |
| 21 | 10 | mg | | | | | | |
| 22 | 7.5 | mg | 2.5 | mg | | | | |
| 23 | 7.5 | mg | 2.5 | mg | 200 | μg | | |
| 24 | 7.5 | mg | 2.5 | mg | 400 | μg | | |
| 25 | 7.5 | mg | 2.5 | mg | 1 | mg | | |
| 26 | 7.5 | mg | 2.5 | mg | 1.5 | mg | | |
| 27 | 10 | mg | 2.5 | mg | | | 400 | μg |
| 28 | 7.5 | mg | 2.5 | mg | | | 400 | μg |

For Expmt #25-26, the liposomes demonstrated efficient RNA binding ability and transfection efficiency. However no IO nanoparticles were detected within the liposomes. For Expmt #27-28, the liposomes demonstrated poor transfection efficiency.

In a fifth series of experiments, liposomes were mixed with iron oxide in chloroform (Expmt 30-31) and rehydrated with 4 mL room temp PBS or left without iron oxide in the chloroform phase and rehydrated with 2.5 mg commercially-available IO nps in 100 μL PBS. All samples were sonicated for 1 hr at a starting temp of 55° C. and then left at room temperature overnight before filtering through 450 nm Whatman and 200 nm PALL filters. Table 5 details the materials used.

TABLE 5

| Expmt | | | IO | |
|---|---|---|---|---|
| # | DOTAP | Cholesterol | nanoparticles | Procedure |
| 29 | 1.8 | 0.6 | 2.5 mg | Rehydrated with 100 μL |
| | | | | IO with 900 μL PBS |
| 30 | 7.5 | 2.5 | 10 mg (oleic | Sonicated 1 hr in |
| | | | acid coated) | chloroform with IONPs |
| 31 | 7.5 | 2.5 | 10 mg (oleic | Added IONPs slowly to |
| | | | acid coated) | lipids in chloroform, |
| | | | | evaporating in 20 μL |
| | | | | increments under N₂. |

For Expmt #29, iron oxide was detected outside of the liposomes. For Expmt #30-31, massive aggregates of iron oxide were detected.

In a sixth series of experiments, a lipid cake comprising 7.5 mg DOTAP and 2.5 mg cholesterol was rehydrated with a solution comprising IO nanoparticles. In one experiment, 2 mg of carboxylated IO nanoparticles (10 nm diameter) were used. In three experiments, varying amounts of 130 nm carboxylated iron oxide nanoparticles (NanoMag) were used to make liposomes with varying amounts of iron oxide within the liposome core. Liposomes formed by rehydrating lipid cakes with 2 mg carboxylated 10 nm iron oxide nanoparticles or 10 μg, 100 μg, or 1 mg NanoMag were tested for RNA binding and transfection efficiency following protocols as described in Sayour et al., Oncoimmunology 6(1): e1256527 (2015). All four sets of liposomes bound RNA and were able to transfect cells. Liposomes with a 1 mg IONPs demonstrated the highest level of transfection efficiency. These liposomes were shown to have IO inside the liposomes by Cryo-TEM and advantageously, these liposomes stimulated anti-tumor immunity in tumor models.

### Example 4

This example demonstrates exemplary methods of making magnetic liposomes and the characterization thereof.

Cationic liposomes were created using a variation of the thin film rehydration technique described in Example 1. A schematic of the basic steps are shown in FIG. 7A. In one variation of the technique, iron oxide nanoparticles (IONPs) are added at Step 3. Briefly, 7.5 mg DOTAP and 2.5 mg cholesterol were dissolved in chloroform and added to a borosilicate glass tube (Step 1). Chloroform was evaporated in N₂ (Step 2) and allowed to dry for one hour before rehydration with a dense iron oxide solution (200 μL of 5 mg/mL NanoMag®) (Step 3). Particles were then incubated in a water bath at 50° C. and vortexed every 10 minutes for 1 hour to allow liposome formation. Liposomes were then left overnight at room temperature. The next day, liposomes were sonicated for 5 minutes at room temperature and filtered through 450 nm and 200 nm filters (Step 4).

In a second variation of the technique, IO was added at Step 1. Briefly, 7.5 mg DOTAP and 2.5 mg cholesterol were dissolved in chloroform along with oleic acid coated iron oxide and added to a borosilicate glass tube (Step 1). Chloroform was evaporated in N₂ (Step 2) and allowed to dry for one hour before rehydration with PBS (Step 3). Particles were then incubated in a water bath at 50° C. and vortexed every 10 minutes for 1 hour to allow liposome formation. Liposomes were then left overnight at room temperature. The next day, liposomes were sonicated for 5 minutes at room temperature and filtered through 450 nm and 200 nm filters (Step 4).

It was found that the most effective nanoparticles were made by the method comprising the step of adding carboxylated iron oxide nanoparticles during the rehydration step (Step 3), wherein a dense, ≥1 mg/mL solution of iron oxide nanoparticles (instead of PBS) was used as the rehydration material. The process comprising the addition of carboxylated iron oxide nanoparticles at Step 1 also produced effective nanoparticles, though to a lesser extent, relative to when IO was added at Step 3.

Magnetic liposomes produced by the above method were loaded GFP RNA at 5 μg RNA:75 ug lipid ratio. The liposomes were analyzed with a Nanosight NS300 instrument. These magnetic liposomes were shown to have IO inside the liposomes by Cryo-TEM (FIG. 7B) and appeared to be mainly in the range of 100 nm to 300 nm, with another bump at 370 likely indicating aggregation of multiple particles (FIG. 7C). Thus, it was confirmed that the magnetic liposomes were indeed nanoparticles (NPs). Throughout the present disclosure, iron oxide-RNA nanoparticles (IO-RNA-NP) are synonymous with magnetic liposomes.

IO-RNA-NPs produced by the above method, wherein ≥1 mg/mL solution of IONPs was used as the rehydration material, or by methods described in Example 3, were loaded with GFP RNA at 5 μg RNA:75 μg lipid ratio. Each liposome was added to a well of a 1% agarose gel. As shown in FIG. 7D, there was an absence of bands in each condition indicating that all methods resulted in liposomes that bound 100% of loaded RNA at these conditions.

RNA-NPs with and without iron oxide were used to transfect DC2.4s with GFP RNA. IO-RNA-NPs exhibited significant transfection efficiency that was greater than the transfection efficiency of standard RNA-NPs without iron oxide (FIG. 7E)

RNA-NPs loaded with JO and Cholesterol (Chol-IO-RNA-NPs) were used to transfect DC2.4s and compared to standard IO-loaded RNA-NPs (with 0% cholesterol). GFP+ cells were measured by flow cytometry. As shown in FIG. 7F, the presence of cholesterol enhanced transfection efficiency by more than 2-fold, relative to IO-loaded NPs without any cholesterol. This result indicates that a 3:1 ratio of DOTAP to cholesterol provides optimal transfection efficiency for liposomes with iron oxide loading. Bright field images of the transfected DCs were taken (FIG. 7G). Fluorescent imaging of the transfected DCs were taken (FIG. 7H) and demonstrated the presence of Cy3-labeled RNA in the perinuclear area of the DCs. The localization to this area seemed to occur regardless of iron oxide or cholesterol content.

IO-loaded liposomes were made as described above except with varying amounts of carboxylated JO (130 nm diameter). Lipids (10 mg 7.5 DOTAP and 2.5 mg cholesterol) were rehydrated in choloroform with varying amounts of carboxylated iron oxide (200 μg, 400 μg, 1 mg, or 1.5 mg) to yield particles with final iron oxide content of 0.5 μg/uL, 1 μg/uL, 2.5 μg/uL or 3.75 μg/uL. These particles were loaded with Cy5-labelled GFP RNA and used to transfect DC2.4s. Flow cytometry at 24 hours indicated that increasing iron oxide content increases transfection efficiency (FIG. 7I). This enhancement has never been reported for any cell type and this observation may support a new use of iron oxide loaded liposomes for enhancing transfection of cells, e.g., DCs.

### Example 5

This example demonstrates the effect of a magnetic field on magnetic liposomes.

The effect of a 101 mT magnetic field on the ability of magnetic liposomes to deliver RNA to cells was tested. Magnetic liposomes with either 0% or 25% cholesterol were loaded with GFP RNA as described in Example 1. The GFP RNA-loaded magnetic liposomes were incubated with DC2.4 dendritic cells for 30 minutes in the presence or absence of a magnetic field. For one set of cells, the RNA-loaded magnetic liposomes were incubated with DC2.4 dendritic cells overnight in the absence of a magnetic field produced by a MagneFect-Nano II 24 well magnet array. After 30 minutes, particle-containing media was removed and replaced with fresh media. Gene delivery was assessed as GFP expression by flow cytometry at 24 hours. As shown in FIG. 8A, the number of GFP⁺ DCs was higher when a magnetic field was present, relative to when a magnetic field was absent. These data support that magnetic fields can be used to enhance RNA delivery to DCs.

Cholesterol-loaded magnetic liposomes synthesized with varying amounts of iron oxide (0 μg, 10 μg, 100 μg 1000 μg) per 10 mg lipid were loaded with Cy5-labelled GFP RNA. The RNA-loaded magnetic liposomes were then used to transfect DC2.4 dendritic cells for 30 minutes in the presence of a static magnetic field. As shown in FIG. 8B, the presence of iron oxide in the liposome increased the % GFP⁺ DCs in a dose dependent manner. The lowest amount of IO yielded a 2-fold difference in % GFP⁺ DCs, while the highest amount of IO led to a 5-fold increase in % transfected cells. These data support that iron oxide content enhances responsiveness to magnetic fields and subsequent transfection of dendritic cells.

Magnetic liposomes (with 1 mg IO per 10 mg lipid) were loaded with Cy3-labelled RNA and subsequently incubated with DC2.4 dendritic cells for 30 minutes in the presence or absence of a magnetic field or overnight in the absence of a magnetic field. Flow cytometry was conducted 24 hours after magnetic exposure to determine the % of GFP⁺ cells. As shown in FIG. 8C, the % GFP⁺ cells was highest for cells that had an overnight (18-hour) exposure to IO-deficient liposomes in the absence of a magnetic field. However, the same level of transfection could be achieved in only a small fraction of the time (30 minutes) when the liposomes comprised IO and were exposed to a magnetic field for the duration of the RNA-cell incubation period. These results suggest that the transfection efficiency for magnetic liposomes was 6 times higher in the presence of a magnetic field. The substantial reduction in time (30 minutes vs. 18 hours) is significant because overnight incubation with cationic liposomes is associated with cytotoxicity. Thus, by reducing the RNA-cell incubation time by more than 95%, it is thought the number of transfected cells produced per patient would increase.

The presence of iron oxide inside cells provides the additional opportunity to manipulate cells with a magnetic field. One use of this technique would be to differentiate cells that did and did not take up iron oxide nanoparticles. To test this application, BMDCs were incubated with RNA-loaded magnetoliposomes overnight as described above. After 18 hours, DCs were transferred to an Eppendorf tube and placed on a magnetic separator for 30 minutes at 37° C. As seen in FIG. 8D, a visible mass of BMDCs was attracted to the side of the Eppendorf tube where the magnetic field strength was greatest.

Primary bone marrow derived dendritic cells (BMDCs) are more representative of patient-derived dendritic cells used in clinical studies. In one study, the effect of a magnetic field on cell transfection efficiency using magnetic liposomes were tested using primary BMDCs. Liposomes comprising DOTAP and cholesterol at a 3:1 DOTAP:Cholesterol ratio with or without 1 mg (10% by mass) IO were loaded with Cy5-labelled GFP RNA. RNA-loaded liposomes were then incubated with primary BMDCs for 30 minutes in the presence or absence of a magnetic field or overnight in the absence of a magnetic field. As shown in FIG. 8E, the % of labeled BMDCs was higher when cells were incubated with RNA in the presence of a magnetic field. GFP expression assessed by flow cytometry and, as shown in FIG. 8F, primary BMDCs cells transfected with liposomes comprising IO in the presence of a static magnet led to an almost 2-fold increase in % GFP+ cells, relative to cells transfected with liposomes lacking IO or comprising IO but incubated in the absence of a magnet. These results indicate that a 30 minute incubation of iron oxide loaded nanoparticles in the presence of a magnetic field is sufficient to achieve a 100% increase in transfection efficiency compared to an overnight incubation in the absence of that field.

### Example 6

This example demonstrates that RNA-loaded magnetic liposomes stimulate DC activation and migration to lymph nodes.

Dendritic cells are important components of the antiviral immune response. One of the main roles of these cells is to produce Type I Interferon (IFN-α or IFN-β) in response to the sensation of foreign nucleic acids, which are characteristic of viral infection. These antigen-experienced DCs then present viral antigens to CD8⁺ T cells. T cells that bind antigen on DCs in the presence of costimulatory molecules are stimulated to release IFN-γ. A diagram of these pathways is found in FIG. 9A.

With these cytokine stimulation pathways in mind, we measured IFN-α release by DCs incubated with GFP RNA-loaded liposomes (GFP mRNA+NPs), DCs electroporated with GFP RNA (electro), DCs incubated with liposomes lacking RNA loading, and untreated DCs at a ratio of 1.33 μg RNA:25 μg liposomes:200,000 cells in a 24 well plate. Supernatants were collected at 20 hours after the start of incubation and analyzed for production of IFN-alpha with an ELISA. As shown in FIG. 9B, the level of IFN-α was highest when DCs were incubated with GFP-RNA loaded liposomes This increase in IFN-alpha production achieved with magnetic liposomes is clinically relevant, as it has been shown to be essential to antitumor immune responses in similar vaccination models.

In order to test T cell stimulatory capacity of RNA-NP loaded DCs, we incubated ovalbumin-specific OT1 transgenic T cells with BMDCs loaded overnight with magnetic liposomes bearing ovalbumin RNA. Supernatants were removed after 48 hours and an ELISA was run to evaluate IFN-gamma release from these cells. As shown in FIG. 9C, BMDCs loaded with magnetic liposomes induced T cell activation.

Previous studies in humans with glioblastoma indicate that migration of RNA-pulsed dendritic cells to lymph nodes correlate directly with successful immunization (Mitchell et al., Nature 519: 366-369 (2015)). Therefore, we sought to evaluate the effect of magnetoliposomes on migratory capacity of dendritic cells relative to the current gold standard DC transfection technique (electroporation). Naïve OT1 (18 hour time point) or C57B16 mice (48 and 72 hour timepoints) received intradermal injection of DsRed DCs loaded with OVA RNA (18 hours) or Cy3-labelled GFP RNA (48 hours and 72 hours) via electroporation or via magnetic liposomes on opposite sides. Bilateral lymph nodes were harvested at 18, 48, or 72 hours and dissociated with collagenase. Flow cytometry was then run to quantify the number of cells that migrated to each lymph node in each animal. As shown in FIG. 9D, dendritic cells loaded with magnetic liposomes enhanced migration to lymph nodes (LN) by >80% on Day 2 and >25% on Day 3, relative to DC migration to LN when DCs were electroporated with RNA. This enhanced migratory capacity suggests that magnetoliposome-loaded DCs are fundamentally distinct from electroporated DCs and may provide unique benefits to antitumor immune responses.

This example demonstrated that magnetic liposomes can enhance DC activation.

### Example 7

This example demonstrates that magnetic liposomes enable MRI-based cell tracking.

Magnetoliposomes were loaded with OVA RNA and then incubated with BMDCs. These DCs were then injected intradermally into the left inguinal area of C57B16 mice. After 48 hours, these mice were imaged with an 11T MRI using with various MRI imaging sequences. These sequences included T2* weighted sequences with Repetition Time (TR)/Echo Time (TE) ratios of 90/3 with and without fat saturation, 3500/12 with fat saturation, 207/17 with fat saturation, and 90/5 with fat saturation, and T2 RARE weighted sequences with TR/TE of 500/14 with and without fat saturation. Regions were drawn around lymph nodes in all slices of these images sequences. Changes in intensity between the treated and untreated lymph nodes in T2* weighted images with a TR/TE of 207/17 and T2_RARE weighted images for each set of imaging parameters are displayed with representative images in FIG. 10A and quantified in FIG. 10B. The images and data demonstrate a reduction in signal intensity of T2* weighted images in the treated lymph nodes, which is an expected effect of iron oxide.

After imaging, lymph nodes were harvested and analyzed with flow cytometry for DsRed⁺ cells. The number of cells in each lymph node was then plotted against the relative change in intensity in T2*-weighted images with fat saturation (FIG. 10C) and the average relative change in lymph node size compared to a non-treated lymph node across the different imaging sequences mentioned above (FIG. 10D). When determining the average increase in lymph node size, the volume of each inguinal lymph node (treated and untreated) was found by drawing regions of interest around each lymph node in each MRI slice. ImageJ was then used to calculate the area of each of those slices. These areas were multiplied by the width of each slice to determine the volume of that slice. These volumes were then summed for all slices in that image set that contained a lymph node to generate an MRI-detected lymph node volume for each imaging sequence. The relative change in lymph node size for each sequence was then found by dividing the volume of the treated lymph node by the volume of the contralateral untreated lymph node for each sequence. The overall average change in lymph node size was then found by taking the average of all calculated relative changes in lymph node volumes across the 7 imaging sequences described above.

As shown in FIGS. 10C and 10D, there was a clear quantitative relationship between the number of iron oxide loaded dendritic cells in each lymph node and the intensity of the T2*-weighted MRI image (10C) and lymph node size (10D). The correlation appeared to be surprisingly reliable. Previous attempts to quantify dendritic cell migration to lymph nodes required relatively complex calculations of signal to noise ratio or comparison of intensity within a “void volume” (de Chickera S, et al., Int Immunol. 2012; 24(1):29-41. doi: 10.1093/intimm/dxr095. PubMed PMID: 22190576; and Zhang et al., Radiology 274(1): 192-200 doi:10.1148/radio1.14132172)

The observation that T2*-weighted MRI intensity and lymph node size both correlate directly with the number of magnetoliposome-loaded dendritic cells in vaccination site draining lymph nodes is a significant advance, because it allows a relatively naïve observer to evaluate success of a vaccination strategy with very simple analytic techniques. Additionally, while others have shown a relationship between dendritic cell migration and MRI output, none of these have utilized a multifunctional particle that could deliver antigen to DCs, activate those DCs, and enable MRI-based tracking of those cells. Therefore the magnetic liposomes described herein are the first multifunctional particles having the ability to (1) deliver RNA to DCs with a high efficiency, (2) activate DCs to release IFNα, (3) enable enhanced transfection efficiency with application of external magnetic fields, and (4) generate sufficient changes in the MRI signal to semi-quantitatively evaluate dendritic cell migration to lymph nodes. The line of best fit was produced using linear regression and a Pearson's correlation was used to calculate the goodness-of-fit, displayed here as a p value evaluating the relationship between cells counted and MRI intensity.

### Example 8

This example demonstrates MRI-detected DC migration predicts inhibition of tumor growth.

C57B16 mice were given B16F10-OVA tumors via subcutaneous administration of 1 million cells on Day 0. On the same day mice received intradermal injection of 500,000 bone marrow derived dendritic cells loaded with magnetic liposomes bearing ovalbumin (OVA)-encoding RNA at a ratio of Mug RNA to 150 μg magnetoliposomes for every 2 million cells and intravenous injection of 10 million OT1 T cells. Mice were imaged with an 11T MRI after 2 days using the unique set of six MRI sequences described in Example 7.

In a first experiment, C57B16 mice with tumors were either treated as above or left untreated. Tumor growth was measured for more than 20 days and the results are shown in FIG. 11A. As shown in this figure, tumor size was substantially less for mice treated with BMDCs loaded with magnetic liposomes comprising OVA RNA, relative to untreated mice.

In a second experiment, C57B16 mice with tumors were treated as above and tumor growth was measured for 30 days (FIG. 11B). A subset of mice whose tumors grew rapidly were designated as “Nonresponders” and a subset whose tumors did not grow were designated as “Responders”. As shown in FIG. 11C, Responders and Non-responders had significantly different tumor volumes on Day 27. FIG. 11D shows an Analysis of the Day 2 MRI sequences of these mice. As shown in FIG. 11D, MRI sequences indicate a significant difference in the T2*fatsat image intensity between the two groups. The T2*fatsat sequence that we used for this analysis is unique. Six other sequences that are theoretically similar were evaluated but none provided a quantitative relationship between MRI intensity on Day 2 and tumor growth. See, for example, FIG. 11E. Day 27 tumor volumes were plotted against Day 2 MRI intensity to indicate a significant linear relationship. This is depicted in FIG. 11F.

These data suggest that magnetic liposomes enable MRI-based detection of dendritic cell migration two days after treatment that accurately predicts which mice will respond to treatment four weeks after treatment. This is the first demonstration of correlation of MRI intensity with inhibition of tumor growth with a multifunctional particle. A predictive effect of MRI has only been shown by one other group (Zhang Z, Li W, Procissi D, Li K, Sheu A Y, Gordon A C, Guo Y, Khazaie K, Huan Y, Han G, Larson A C. Antigen-loaded dendritic cell migration: MR imaging in a pancreatic carcinoma model. Radiology. 2015; 274(1):192-200. doi: 10.1148/radio1.14132172. PubMed PMID: 25222066; PMCID: PMC4314117). However, this group used a less direct approach that relied on signal to noise ratio in before-and-after images. This group also did not use a multifunctional particle. The simplified method described herein allows for quantification and comparison to unvaccinated lymph nodes of the same animal using a simple but unique MRI sequence.

### Example 9

This example demonstrates a method of treating a human patient with DC vaccines comprising a magnetic liposome of the present disclosure and a method of predicting response to treatment.

Iron oxide loaded magnetic liposomes are made as essentially described in Example 7B. A tumor is excised from a cancer patient and RNA is isolated from tumor cells. The RNA is then expanded through reverse transcription into a cDNA library, expansion of that cDNA library with PCR, and in vitro transcription and loaded into the magnetic liposomes as essentially described in Example 8. In another embodiment, RNA encoding a specific tumor-associated antigen are produced and loaded into magnetic liposomes as essentially described in Example 8. White blood cells are isolated from the same cancer patient via leukapheresis. The WBCs are treated with IL-4 and GM-CSF to generate dendritic cells. The DCs are loaded with magnetic liposomes prepared with RNA as essentially described in Example 8. About 2×10⁷ dendritic cells comprising the magnetic liposomes with RNA are injected intradermally into the patient's groin. Various MRI images including T2*-weighted images with echo times of 5 ms are taken immediately before vaccination and 24, 48, and 72 hours after vaccination.

The MRI's obtained before and after treatment are used to determine the change in lymph node size and T2*-weighted lymph node intensity for each patient. Regions of interest are then drawn around each inguinal lymph node for processing with ImageJ as described in Example 8. Briefly, average lymph node volume across 7 imaging sequences and the average intensity inside lymph nodes on T2*-weighted images 1, 2 and 3 days after treatment is plotted against progression free and overall survival for patients with malignant glioma. A strong correlation between lymph node size or hypointensity with progression free or overall survival is then used to predict patient response to treatment within days of vaccination.

### Example 10

This example demonstrates a method to deliver immune modulatory nucleic acids to immune cells in the periphery and within malignant brain tumors.

Patients with a malignant brain tumor undergo surgical resection or biopsy of the primary tumor site. RNA from that tumor is isolated and expanded as described in Example 9. The expanded mRNA encoding tumor antigens is conjugated to liposomes composed of around 75% DOTAP and 25% cholesterol at a ratio of 25 ug RNA:375 ug liposome. In another embodiment, the mRNA includes mRNA specific for a tumor associated antigen, which optionally include the cytomegalovirus antigen pp65. In another embodiment, the RNA would include mRNA encoding for immune modulatory proteins, for example a costimulatory molecule like CD86, a chemoattractant like CCL3, or an activating cytokine like granulocyte-monocyte colony stimulating factor (GM-CSF). In another embodiment, the RNA includes siRNA or shRNA designed to modulate immune function of transfected cells. An example of an immune modulatory target for siRNA or shRNA delivery is programmed death ligand 1 (PD-L1). In another embodiment, the RNA includes other nucleic acids useful for activating or inhibiting immune function. In another embodiment, the RNA includes two or more of the RNA constructs listed above in a combination therapy. For example, mRNA derived from the tumor is combined with pp65 mRNA with or without an siRNA for PDL1. In another embodiment, the liposomes include 1 mg iron oxide per 10 mg lipid. These liposomes are then injected intravenously into the patient at regular intervals. In one embodiment, this interval is twice weekly for six injections and then twice monthly for six more injections or until disease progression. In another embodiment, patients receive different RNA constructs in sequential vaccines. For example, patients receive liposomes loaded with tumor-derived mRNA for three weeks, and then tumor-derived mRNA and PDL1 siRNA for the next six bimonthly injections.

For patients vaccinated with liposomes containing iron oxide, T2*-weighted MRI sequences are taken before and 24 hours after vaccination. Changes in MRI intensity before and after vaccination are plotted against progression free and overall survival to evaluate whether MRI-detected particle localization to brain tumors correlates with patient response to treatment.

### Example 11

This example demonstrates the effects of lipid composition on particle localization and the characterization of RNA-loaded cells.

We previously reported that cationic liposomes loaded with mRNA encoding tumor antigens (RNA-NPs) induced robust immune activation in peripheral organs sufficient to eliminate subcutaneous and intracranial tumors in preclinical models of melanoma^{9,10}. However, these particles largely accumulate in the lung due to their positive charge (FIG. 24)⁹. In order to avoid this pulmonary uptake, we modified the lipid composition to include cholesterol in an attempt to reduce the concentration of positive charge on the liposome surface. This modification failed to alter particle localization to lungs and spleens, but did increase RNA-NP localization to livers (FIG. 23). We then tested particle localization in mice with intracranial KR158b-Luciferase, a radiation, chemotherapy, and checkpoint inhibitor resistant murine glioma line. Surprisingly, immunofluorescence microscopy 24 hours after liposome injection revealed that these modified liposomes accumulated in brain tumors, but not the surrounding normal cortex (FIG. 12A). However, liposomes without the cholesterol content failed to accumulate in either area.

Intrigued by this result, we repeated this observation in a larger cohort of animals using flow cytometry to quantify particle uptake in KR158b-Luciferase and GL261. We found that the presence of cholesterol enhanced liposome uptake in both tumors (FIG. 12B-C), and that this effect was most pronounced at a cholesterol concentration of 25% by mass (FIG. 12D). Taken together, this evidence indicates that cholesterol-bearing liposomes may be a simple tool to direct immunomodulatory nucleic acids to brain tumors.

We then sought to characterize the RNA-loaded cells in brain tumors. Since the liposomes accumulated only in tumors and not in normal brain, we hypothesized that they could be infiltrating the tumor-associated vascular endothelium. Although the RNA-loaded cells did localize near the brain tumor endothelium, both immunofluorescence microscopy and flow cytometry demonstrated that these cells lacked CD31 (FIG. 2A). However, in contrast to the bulk tumor which was about 50% CD45+, RNA-labelled cells in both GL261 and KR158b were almost 100% CD45+(FIG. 13B, C). Additionally, RNA-labelled CD45+ cells in KR158b and GL261 were disproportionately F4/80+, MHCII+, CD11b+ and Ly6G/6C+(FIG. 13D-G). This intriguing result suggests that Chol-RNA-NPs are delivering nucleic acids directly to antigen presenting cells in the brain tumor microenvironment consist with tumor associated macrophages (TAMs).

### Example 12

This example demonstrates the effects of Chol-RNA-NPs on RNA-loaded cells.

TAMs are known to suppress antitumor immune responses, but can be reprogrammed to an antitumor phenotype characterized by increased expression of MHCII, CD80, and CD86 (^{6,11}). Since we previously demonstrated that RNA-NPs activate innate immune cells in peripheral organs, we next evaluated whether these liposomes could also activate TAMs in the brain tumor microenvironment. We again loaded liposomes with fluorescently tagged mRNA and injected these systemically into mice. After 24 hours, we found that the F4/80+ cells that had taken up Chol-RNA-NPs significantly upregulated expression of MHCII (FIG. 14A). Furthermore, we found that these Cy5-labelled MHCII+ cells also expressed high levels of CD80 and CD86 (FIG. 14B, C). Interestingly, we also found an increase in CD80 on TAMs in the bulk population that did not contain RNA.

### Example 13

This example demonstrates the delivery of PDL1 siRNA with Chol-RNA-NPs.

To test this hypothesis, we sought to combine Chol-RNA-NPs with cell-specific checkpoint blockade. We first generated a library of anti-PDL1 siRNAs from published literature and screened them for the ability to abrogate PDL1 expression in DCs in vitro on days 2, 3, and 4 after transfection without compromising transfection efficiency. Through this screen we identified an siRNA species that significantly reduced PDL1 expression without reducing GFP expression (siPDL1) (FIG. 24).

We then tested whether Chol-RNA-NPs could deliver this siRNA to intracranial brain tumors. In support of our results delivering mRNA, we found that Chol-RNA-NPs significantly increased delivery of Cy3-labelled siRNA to CD45+MHCII+ cells in intracranial tumors (FIG. 15 A-C). Similar to the cells loaded with mRNA, many of these cells expressed APC markers MHCII, F4/80, and CD11b (FIG. 15 D-F).

We then tested whether the siRNA delivered by Chol-RNA-NPs was functionally active by staining for PDL1 expression on these cells. For these experiments, we utilized a noncoding siRNA as a control (siCTRL). We found that a single injection of Chol-RNA-NPs bearing siPDL1 reduced PDL1 expression on antigen presenting cells loaded with siRNA compared to cells in untreated tumors and to the bulk of cells in treated tumors (FIG. 15 G-I). We then demonstrated that this effect could be magnified with additional administrations. We found that three daily administrations of siPDL1 via Chol-RNA-NPs dramatically reduced PDL1 expression on transfected MDSCs compared to untreated cells (Untreated: 60.06+/−22.24; Cy5+ siPDL1: 16.7+/−19.23; p=0.0255), the bulk cells in the treated tumor (Bulk: 45.82+/−17.04; Cy5+ siPDL1: 16.7+/−19.23; p=0.047), and cells treated with the control siRNA (CTRL: 77.14+/−28; Cy5+ siPDL1: 16.7+/−19.23; p=0.0081) (FIG. 15J). Taken together, this evidence indicates that Chol-RNA-NPs can be used to activate intratumoral immune cells and modify cell behavior with immune-regulatory nucleic acids (FIG. 15K).

### Example 14

This example demonstrates the materials and methods used in the experiments of Examples 11-13.

RNA Preparation and Labeling: GFP and OVA mRNA were generated via in vitro transcription as previously described¹². PDL1 and CTRL siRNA were purchased from Santa Cruz Biotechnology. Nucleic acid labelling was completed with Arcturus Turbo Labeling Kits according to manufacturer instructions (ThermoFisher Scientific).

Cell culture: DC2.4s and KR158b-Luciferase were a kind gift from John Sampson, Duke University¹³. GL261 was purchased from EMD Millipore. DC2.4s and KR158b-Luciferase were cultured at 37° C. with 5% CO₂ in high glucose DMEM with pyruvate supplemented with 10% heat inactivated fetal bovine serum (FBS) and 1% Penicillin/Streptomycin (LifeTechnologies). GL261 was cultured in DMEM/F-12 with Glutamax, 10% FBS, and 1% Penicillin/Streptomycin (LifeTechnologies).

Synthesis of RNA-NPs: (1) Liposome synthesis: Liposomes were prepared as previously described¹². Briefly, dehydrated DOTAP and Cholesterol 700000P (Avanti Polar Lipids) were suspended in chloroform, mixed at DOTAP:Cholesterol ratios of 4:0, 3.5:0.5, 3:1, or 2.5:1.5. Chloroform was then evaporated in the presence of nitrogen. The lipid cake was then brought to a concentration of 2.5 mg/mL in phosphate buffered saline (PBS) and heated at 50 C while vortexing every ten minutes for one hour and left at room temperature overnight. The lipids were then sonicated five minutes and filtered through 450 and 200 nm filters (Whatman and PALL, respectively). (2) RNA-NP complex formation: Chol-RNA-NPs were prepared as described previously for RNA-NPs¹². Briefly, 375 ug lipids were combined with 25 ug mRNA or siRNA per mouse and allowed to incubate 15 minutes before injection.

In vitro transfection: DC2.4s were plated at 100,000 cells per well in a 24 well plate. After 24 hours, RNA-NPs were added to the wells at 1.667 ug mRNA per well. Transfection was evaluated with flow cytometry at 24, 48, 72, and 96 hours.

Mice: C57Bl/6, mice were purchased from Jackson Laboratories Animal procedures were approved by the University of Florida Institutional Animal Care and Use Committee (UFIACUC201607966).

Flow cytometric analysis: Flow cytometry was performed using the BD Biosciences FACS Canto-II using antibodies from BD Biosciences, Biolegend, and Invitrogen. For in vitro experiments, DCs were harvested, washed with PBS, and stained for 20 minutes. Samples were then washed twice with PBS and suspended in FACS buffer. Cell counts and viability were assessed with Vi-Cell XR Cell Viability Analyzer (Beckman Coulter). For in vivo experiments, lymph nodes were harvested into cold PBS, diced with razor blade and digested in papain for 20 minutes at 37 C before filtering through a 70 μm cell strainer, washing with PBS, and staining for 20 minutes with appropriate antibodies. Counting beads were added to each tube immediately before flow analysis.

Immunofluorescence: Mice with intracranial brain tumors were sacrificed and perfused with PBS and 4% formalin 24 hours after injection of fluorescently labelled RNA-NPs. Brains were then harvested, fixed in 4% formalin, transferred to a 4% sucrose solution overnight, and then stored in PBS. Slices were completed with a Leica RM2235 Microtome. Images were taken with an Olympus IX70 Inverted Fluorescent Microscope.

Statistical analysis: Data are presented as the mean±standard deviation. Student's t tests are used for statistical analysis. Paired t tests are used for paired data. Analysis was conducted using GraphPad Prism version 8.

References Cited The following references are cited throughout Examples 11-14: (1) Hilf, N., et al. Actively personalized vaccination trial for newly diagnosed glioblastoma. Nature 565, 240-245 (2019); (2) Schumacher, T. N. & Schreiber, R. D. Neoantigens in cancer immunotherapy. Science 348, 69-74 (2015); (3) Grobner, S. N., et al. The landscape of genomic alterations across childhood cancers. Nature 555, 321-327 (2018); (4) Charles, N. A., Holland, E. C., Gilbertson, R., Glass, R. & Kettenmann, H The brain tumor microenvironment. Glia 59, 1169-1180 (2011); (5) Lewis, C. E. & Pollard, J. W. Distinct role of macrophages in different tumor microenvironments. Cancer Res 66, 605-612 (2006); (6) Poon, C. C., Sarkar, S., Yong, V. W. & Kelly, J. J. P. Glioblastoma-associated microglia and macrophages: targets for therapies to improve prognosis. Brain 140, 1548-1560 (2017); (7) Morantz, R. A., Wood, G. W., Foster, M., Clark, M. & Gollahon, K. Macrophages in experimental and human brain tumors. Part 2: studies of the macrophage content of human brain tumors. J Neurosurg 50, 305-311 (1979); (8) Abbott, N.J., Ronnback, L. & Hansson, E. Astrocyte-endothelial interactions at the blood-brain barrier. Nat Rev Neurosci 7, 41-53 (2006); (9) Sayour, E. J., et al. Systemic activation of antigen-presenting cells via RNA-loaded nanoparticles. Oncoimmunology 6, e1256527 (2017); (10) Sayour, E. J., et al. Personalized Tumor RNA Loaded Lipid-Nanoparticles Prime the Systemic and Intratumoral Milieu for Response to Cancer Immunotherapy. Nano Lett (2018); (11) Mosser, D. M. & Edwards, J. P. Exploring the full spectrum of macrophage activation. Nat Rev Immunol 8, 958-969 (2008); (12) Sayour, E. J., et al. Systemic activation of antigen presenting cells via RNA-loaded nanoparticles. Oncolmmunology, 00-00 (2016); (13) Shen, Z., Reznikoff, G., Dranoff, G. & Rock, K. L. Cloned dendritic cells can present exogenous antigens on both MHC class I and class II molecules. J Immunol 158, 2723-2730 (1997).

### Example 15

This example demonstrates Dendritic Cell (DC)-activating magnetic nanoparticles enable early prediction of anti-tumor response with MRI.

Abstract: Cancer vaccines initiate antitumor responses in a subset of patients, but the lack of clinically meaningful biomarkers to predict treatment response limits their development. Here, we design multifunctional RNA-loaded magnetic liposomes to initiate potent antitumor immunity and function as an early MRI-based imaging biomarker of treatment response. These particles activate DCs more effectively than electroporation leading to superior inhibition of tumor growth in treatment models. Inclusion of iron oxide enhances DC transfection and enables tracking of DC migration with MRI. We show that T2*-weighted MRI hypointensity in lymph nodes is a strong correlate of DC trafficking and is an early predictor of antitumor response. In preclinical tumor models, MRI-predicted “responders” identified two days after vaccination had significantly smaller tumors 2-5 weeks after treatment and lived 100% longer than MRI-predicted “non-responders.” These studies therefore provide a simple, scalable nanoparticle formulation to generate robust antitumor immune responses and predict individual treatment outcome with MRI.

Cancer immunotherapy has produced impressive tumor regression in settings where conventional treatments yield no benefit¹. However, even the most effective immunotherapy strategies extend survival for only a subset of patients²⁻⁶. Although multiple pretreatment biomarkers suggest susceptibility to immunotherapy, there are currently no robust markers that predict clinical response⁷⁻⁹. Future development of promising cancer immunotherapies will require dynamic biomarkers to differentiate responding and nonresponding patients before tumor progression⁹.

We previously demonstrated that DC migration to vaccine-site draining lymph nodes (VDLNs) as assessed by SPECT/CT imaging of Indium¹¹¹-labeled DCs just two days after vaccination may provide an early biomarker of overall survival in GBM patients treated with RNA-pulsed DC vaccines¹⁰. However, radioactive cell labelling for PET and SPECT is cumbersome and not widely available in the clinical setting¹¹. Clinical evaluation of this biomarker will require a widely available method to sensitively track DC migration without additional cell processing. MRI is a widely available imaging modality that has been used to qualitatively track large numbers of cells in humans, but MRI-based quantification of cell migration to lymph nodes remains challenging¹²⁻¹⁸.

Additionally, although electroporation is widely used to deliver RNA to DCs in clinical trials^{10,19-23}, an immune-stimulatory replacement to electroporation could further enhance therapeutic benefit. Nanomaterials are attractive for non-viral mRNA delivery²⁴, but few nanoparticles reach the clinic due to complexity of large-scale clinical grade manufacturing^{7,25,26}. Here, we overcome this limitation with scalable, multifunctional nanoliposomes based on previously translated materials that efficiently transfect DCs with RNA, stimulate profound DC activation, and establish MRI-detected DC migration as a biomarker of antitumor response to DC vaccines (FIG. 16).

In this study, we first screen a library of lipid particles with proven safety profiles in humans to develop an optimized lipid nanoparticle formulation for DC activation in vitro. We then combine these immune-stimulatory RNA-loaded cationic nanoliposomes (RNA-NPs) with the T₂ MRI contrast-enhancing effects of iron oxide nanoparticles (IONPs). The resulting iron oxide loaded RNA-NPs (IO-RNA-NPs) deliver RNA to DCs, activate those DCs, and enable prediction of tumor regression with MRI. We find that IO-RNA-NPs dramatically change gene expression profiles in DCs compared to electroporation, leading to increased expression of costimulatory markers, production of inflammatory cytokines (e.g. IFN-α), and enhanced migration to lymph nodes. Importantly, we also demonstrate that DCs loaded with RNA encoding tumor antigens via IO-RNA-NPs inhibit tumor growth in a treatment model in which RNA electroporated DCs yield no benefit. In contrast to previous work demonstrating qualitative MRI changes with IONP-loaded DCs^{13,27-29}, we then demonstrate that MRI-detected DC trafficking predicts long-term inhibition of tumor growth and survival in murine tumor models. Substantial reduction in T2*-weighted MRI intensity in treated lymph nodes two days after vaccination correlates strongly with reduced tumor size 2-5 weeks after vaccination and predicts a 100% increase in median survival compared to treated mice without this change. Taken together, our findings demonstrate that these DC-activating IO-RNA-NPs stimulate robust inhibition of tumor growth and enable early prediction of antitumor response to DC vaccines with a widely available imaging modality.

Design of immune-stimulatory iron oxide loaded liposomes: We first sought to develop a simple, translatable method to deliver mRNA to DCs and track their movement with MRI. IONPs are attractive MRI-contrast agents due to their proven clinical utility, but present methods to optimize IONPs for RNA delivery in the preclinical setting utilize polymers without proven safety records in humans (e.g. polyethylenimine) Cationic liposomes are attractive agents for mRNA delivery in the clinical setting due to their simple, scalable synthesis and favorable safety profiles in animals and humans³⁰⁻³², but current lipid nanoparticle formulations in clinical evaluation are optimized for targeted mRNA delivery in vivo but not DC activation in vitro^{30,32-34}. In addition, previous attempts to develop cationic liposomes for DC activation in vitro limited evaluation to expression of activation markers instead of functional outcomes (e.g. capacity for transfected DCs to activate antigen specific T cells)³⁵. Here, we created a library of lipid nanoparticles using commercially available materials with established safety profiles in clinical trials⁷ and evaluated their capacity to transfect and activate bone marrow-derived DCs (BMDCs) in vitro using both basic (i.e., transfection efficiency, viability) and functional tests (FIG. 29A). To do this, we developed a scoring system based on BMDC transfection, viability, expression of co-stimulatory markers (CD80, CD86, CD40), and capacity to stimulate antigen-specific T cells in vitro (FIGS. 25A-25E). We found that the inclusion of cholesterol in 1,2-dioleoyl-3-trimethylammonium-propane (DOTAP) liposomes produced the most effective particles for transfection and activation of murine DCs, with Activation Scores 18 times higher than those achieved by DOTAP liposomes without cholesterol (FIGS. 25A-25E).

We then developed a method to incorporate commercially available IONPs into these DC-activating cationic liposomes to enable MRI tracking without significantly increasing synthesis complexity. Since cationic liposomes have positively charged interiors, we reasoned that addition of negatively charged IONPs during particle formation could produce liposomes with solid iron oxide cores. We therefore rehydrated cationic lipids with various concentrations of carboxylated IONPs (0, 1, 10, 100, or 150 ug IONPs per mg lipid) and incubated the resulting liposomes with mRNA to generate RNA-lipoplexes without IONPs (RNA-NPs) or with IONPs (IO-RNA-NPs). Inclusion of IONPs resulted in formation of 100-300 nm liposomes with clear lipid bilayers and solid cores of IONPs evident by Cryo-TEM (FIG. 17a). Consistent with IONP encapsulation within liposomes, particle size (0 ug: 207.9±67.2 nm; 100 ug: 208.7±92.3 nm) and charge (0 ug: 44.1±4.5 meV; 100 ug: 40.2±7.58 meV) remained consistent regardless of IONP content (FIG. 17b, FIG. 29B). However, particles including iron oxide exhibited substantial magnetic properties (Saturation Magnetization=233938A/m) (FIG. 17c).

We then tested whether the inclusion of IONPs would impede RNA binding and delivery. All particles bound 100% of available RNA under experimental conditions (15:1 Lipid:RNA ratio) (FIG. 17d), and particles with increased IONP content demonstrated slightly higher RNA binding capacity in the setting of excess RNA (0 ug: 0.36 μg RNA/μg Lipid; 100 μg: 0.54 μg RNA/μg Lipid; p=0.0010) (FIG. 17e). Importantly, overnight incubation of particles with DC2.4s, an immortalized cell line of DCs³⁶, demonstrated that inclusion of even high amounts of IONPs did not increase toxicity (FIG. 17f) or reduce RNA-delivery (FIG. 17g). Taken together, this evidence suggests that rehydration of cationic lipids in the presence of negatively charged IONPs produces IO-RNA-NPs with the magnetic properties of IONPs and the RNA-binding capacity of cationic liposomes.

IONPs increase DC transfection and activation Next, we sought to confirm that IONPs would not inhibit transfection efficiency and DC activation. Interestingly, fluorescent images and flow cytometry revealed a dose-dependent increase in transfection efficiency for particles with increasing iron oxide content in DC2.4s, an immortalized cell line of DCs, (0 ug: 10.6%; 150 ug: 33.0%, p=0.0009; Pearson's r=0.8696, p=0.054) (FIG. 18a-b) and primary BMDCs (0 ug: 7.0%; 100 ug: 9.9%; p=0.001) (FIG. 18c). However, the addition of unbound IONPs that were not previously complexed with cationic liposomes produced no benefit (RNA-NPs: 7.0%±0.96; RNA-NPs+IONPs: 5.8%±0.05; p=0.244) (FIG. 18c). These results suggest that IONPs increase transfection efficiency but only when incorporated within liposomes.

We then evaluated whether this increase in transfection was accompanied by enhanced DC activation and function. While RNA-NPs slightly increased expression of the costimulatory molecules CD80 and CD86 beyond that achieved with inflammatory cytokines IL-4 and GM-CSF, IO-RNA-NPs further enhanced expression of CD86 and co-expression of both molecules (FIGS. 26A-26C). Given this suggestion that IONPs contribute to a more activated DC phenotype, we evaluated the impact of IONPs on DC function. Bone marrow-derived dendritic cells (BMDCs) were treated with RNA-NPs or IO-RNA-NPs bearing mRNA encoding ovalbumin (OVA) and incubated with naïve OVA-specific OT1 T-cells or antigen-experienced OVA T-cells. While both particle constructs induced substantial T-cell activation as measured by IFN-γ production at 48 hours, inclusion of IONPs within RNA-NPs significantly enhanced activation of antigen-experienced T-cells (RNA-NP DCs: 2611.8±67.06 μg/mL; IO-RNA-NP DCs: 3653.5±216.8 μg/mL; p=0.0014) (FIG. 18d) and priming of naïve T-cells (RNA-NP DCs: 319.0±41.29 μg/mL; IO-RNA-NP DCs: 874.6±23.9 μg/mL; p<0.0001) (FIG. 18e) in an antigen-specific manner (FIGS. 26A-26C). These unexpected results demonstrate that inclusion of IONPs in liposomes enhances BMDC activation and priming of antitumor T-cell responses. We next evaluated whether IONP-mediated DC transfection could be further enhanced with application of external magnetic fields^{37,38}. Addition of magnetic fields enhanced transfection efficiency of DCs in a manner dependent on the concentration of IONPs within liposomes (FIG. 18f), resulting in a fold increase in transfection efficiency at the highest tested concentration of IONPs (−Mag: 5.8±0.7%; +Mag: 10.3±0.9%; p=0.0014) without compromising viability (FIG. 18g-h). Importantly, a thirty-minute incubation with IO-RNA-NPs under the influence of a magnetic field produced double the transfection efficiency compared to overnight incubation with primary BMDCs (Overnight: 5.5±0.97%; 30 min+Mag: 10.3±0.85%; p=0.0010) (FIG. 18h), leading to comparable T-cell priming capacity (Overnight: 3653.5±216.8 μg/mL; 30 min+Mag: 3070.4±536.1 μg/mL; p=0.16) (FIG. 18i). Magnetic fields can therefore be used to significantly reduce transfection time while maintaining high levels of transfection efficiency.

IO-RNA-NPs activate DCs more effectively than electroporation Although liposome-mediated RNA delivery leads to robust DC activation in vivo^{7,30,31}, electroporation remains the preferred technique for delivering RNA to DCs ex vivo in the clinical setting due to its high transfection efficiency (60-80% of BMDCs) compared to other non-viral transfection methods^{10,19-23,39,40}. Indeed, electroporation transfected a greater percentage of BMDCs than IO-RNA-NPs (Electroporation: 81.1%; IO-RNA-NPs: 17.733%; p<0.0001) (FIG. 19a). However, electroporation bypasses pattern recognition receptors that contribute to DC activation by avoiding natural antigen processing in endosomes and phagolysosomes⁴¹⁻⁴³. The clear difference in these transfection methods is visible with fluorescent microscopy of DCs after delivery of Cy3-labelled RNA (FIG. 19b). RNA delivered by IO-RNA-NPs clusters into bright intracellular compartments consistent with endosomes and phagolysosomes, while RNA delivered by electroporation is diluted throughout the cytosol (FIG. 19b). We hypothesized that this natural antigen uptake would allow IO-RNA-NPs to activate DCs more effectively than electroporation. To test this hypothesis, we evaluated the effects of transfection via electroporation and IO-RNA-NPs on DC activation, including gene expression, upregulation of costimulatory molecules, secretion of inflammatory cytokines, cell migration to lymph nodes, and antitumor immune responses.

We first evaluated the impact of each transfection method on gene expression. BMDCs treated with IO-RNA-NPs exhibited significant changes in RNA expression profiles after 24 hours compared to no treatment or electroporation (FIG. 19c), including enhanced expression of gene sets related to antiviral defense, Type I Interferon production, toll-like receptor (TLR) signaling, and antigen processing and presentation (FIG. 27). Although both treatments increased expression of costimulatory molecules by flow cytometry, IO-RNA-NPs induced higher co-expression of these markers (FIG. 19d, FIGS. 28A-28B). We next evaluated whether this activation phenotype was accompanied by increased secretion of IFN-α, which we and others previously showed is required to initiate antitumor immune responses to systemic RNA-NPs^{30,31}. In accordance with our RNA-sequencing data, we found that IO-RNA-NPs increased production of IFN-α (IO-RNA-NP DCs: 13.1±3.4 μg; Untreated DCs: 7.6±1.3 μg; p=0.0369) (FIG. 19e). In contrast, electroporation nonsignificantly decreased IFN-α production relative to controls (Electroporated DCs: 4.45±1.1 μg; Untreated DCs 7.6±1.0 μg; p=0.2879) (FIG. 19e). This result suggests that IO-RNA-NPs stimulate an IFN-α-dependent antitumor immune response that is absent in electroporated cells.

We previously reported in a blinded and randomized pilot clinical trial that enhanced migration of RNA-loaded DC vaccines to VDLNs correlated with improved survival in patients with GBM¹⁰. We therefore compared the migratory capacity of DCs loaded with IO-RNA-NPs or electroporation in a murine model of our clinical protocol in which DCs are prepared in the setting of GM-CSF and IL-4¹⁰. In this experiment, each mouse received contralateral intradermal injections of DsRed+ DCs treated with RNA electroporation or IO-RNA-NPs. In three separate experiments, each evaluating different time points, DCs loaded with IO-RNA-NPs migrated to lymph nodes more efficiently than those treated with electroporation (18 hours: p=0.003, n=3; 24 hours: p=0.0313, n=6; and 72 hours: p=0.16, n=2) (FIG. 19f).

We then evaluated the impact of IO-RNA-NPs and electroporation on inhibition of tumor growth in an established tumor model. We found that a single vaccination with DCs loaded with OVA mRNA via IO-RNA-NPs 5 days after tumor inoculation significantly inhibited growth of subcutaneous B16F10-OVA tumors (IO-RNA-NP vs Untreated: p=0.0057; IO-RNA-NP vs Electroporation: p=0.0044-) (FIG. 19g). In contrast, DCs treated with OVA RNA electroporation provided no treatment benefit (Electroporation vs Untreated: p=0.8) (FIG. 19g).

Taken together, we have demonstrated that IO-RNA-NPs induce robust DC activation characterized by immune-related gene signatures, expression of costimulatory molecules, secretion of inflammatory cytokines (IFN-α), and enhanced migration to lymph nodes. This DC activation is sufficient to inhibit tumor growth in a treatment model in which RNA electroporation yields no benefit. These results suggest that IO-RNA-NPs are a promising alternative to electroporation for DC vaccines.

Cell tracking with MRI We next sought to evaluate the utility of IO-RNA-NPs for DC tracking with MRI. DsRed+ BMDCs were treated with IO-RNA-NPs and injected intradermally into the inguinal areas of naïve mice. After two days, MRI images revealed a visible increase in volume in treated lymph nodes across multiple imaging parameters (FIG. 20a). Flow cytometry (FIG. 20b) revealed that the relative increase in size of the VDLN compared to the contralateral untreated lymph node correlated strongly with absolute counts of DsRed+ cells in treated lymph nodes (r=0.9134; p<0.0001) (FIG. 20c). Furthermore, optimized T₂*-weighted MRI sequences detected reductions in intensity in VDLN compared to contralateral untreated lymph nodes (Relative Intensity of VDLN) consistent with high concentrations of iron oxide. These reductions in MRI intensity correlated strongly with absolute cell counts in treated lymph nodes (r=−0.6842; p=0.0613) (FIG. 20d). This result demonstrates the capacity for MRI to distinguish lymph nodes with high and low concentrations of IONP-loaded DCs.

MRI as an early biomarker of antitumor response in setting of minimal residual disease and established tumor models Having shown that IO-RNA-NPs stimulate robust DC activation and produce consistent changes to MRI intensity in lymph nodes, we evaluated the utility of MRI-detected DC migration as a biomarker to predict antitumor immune response. We first developed a model of minimal residual disease (MRD) in which vaccination produces variable antitumor responses. Intradermal injection of DCs loaded with IO-RNA-NPs bearing OVA mRNA on the same day of tumor implantation significantly inhibited growth of subcutaneous B16F10-OVA tumors compared to untreated controls (Day 19: p=0.0376; Day 23: p=0.0351; Day 25: p=0.0328) (FIG. 21a). However, treated mice demonstrated heterogenous inhibition of tumor growth (FIG. 21b). These differences became pronounced on Day 27, at which a subset of mice had appreciable tumors while others had none (FIG. 21b). Comparison of Day 2 MRI images revealed that “responders” had significantly reduced T2*-weighted MRI intensity in treated versus untreated lymph nodes compared to “non-responders,” consistent with increased concentrations of iron oxide-loaded DCs in treated lymph nodes (r=0.9324; p=0.0209) (FIG. 21c). The predictive value of this biomarker was confirmed in subsequent experiments demonstrating that reduction in T2*-weighted MRI intensity two days after treatment directly correlates with both Day 27 tumor size (r=0.6577; p=0.0542) (FIG. 21d) and overall survival (r=−0.6957; p=0.0374) (FIG. 21e).

We then evaluated whether MRI-intensity in treated lymph nodes could be used to separate treated mice into meaningful groups to predict treatment outcome. We found that mice with high DC migration indicated by MRI-intensities in the bottom 25^{th} percentile had significantly smaller tumors than mice with intensities in the top 75^{th} percentile throughout the analysis (p=0.0131) (FIG. 21f-g) and on individual days including Day 17 (p=0.0086), Day 22 (p=0.0256), and Day 26 (p=0.0118) (FIG. 21h). These results provide strong evidence that MRI-detected DC migration two days after treatment is an early, dynamic biomarker of antitumor immune responses.

Having established utility in an MRD model, we examined the predictive capacity of MRI-detected DC migration in the setting of established tumors. In this experiment, all mice had palpable subcutaneous tumors before treatment with IO-RNA-NP-loaded DCs (FIG. 22a). Here, we observed much faster tumor growth but still recorded heterogenous efficacy in cohorts responding to DC vaccination (FIG. 22b). Correlation of tumor growth curves with MRI taken two days after vaccination again revealed that early decreases in intensity in treated lymph nodes correlated strongly with future inhibition of tumor growth (Day 14 Tumor Size: r=0.7235, p=0.0276; Day 17 Tumor Size: r=0.7323; p=0.0248) (FIG. 22c, FIG. 22d). Mice with high T2*-weighted MRI intensity in treated lymph nodes had substantial tumors, while those with low intensity had none. This evidence suggests that MRI imaging just two days after vaccination is sufficiently sensitive to predict subsequent antitumor immune responses to DC vaccines.

We then continued to observe these animals to determine whether MRI-detected DC migration predicts the more clinically relevant outcome of survival. Remarkably, Day 2 MRI intensity in treated lymph nodes correlated strongly with long-term inhibition of tumor growth (Middle 50% vs Bottom 25^{th} percentile: p=0.0023; Top 75^{th} Percentile vs Bottom 25^{th} percentile: p=0.0001) and survival (r=−0.8557; p=0.0033) (FIG. 22e, 22f). Mice with substantial DC migration to lymph nodes revealed by relative MRI intensity below the 25^{th} percentile on Day 2 lived significantly longer than those with moderate DC migration indicated by MRI intensities in the middle 50^{th} percentile (p=0.0338; log rank analysis) and twice as long as those with a lack of DC migration indicated by relative MRI intensity in the top 75^{th} percentile (p=0.0896; log rank analysis) (Top 25^{th} percentile: 25±7.0 days; Middle 50%: 34±2.049 days; Bottom 25^{th} percentile: 52.5±3.5 days) (FIG. 22g). These results indicate that MRI-imaging of DC trafficking can be used as a highly correlative biomarker to distinguish long-term antitumor responses to IO-RNA-NP-loaded DC vaccines just two days after vaccination.

### Example 16

This example demonstrates the materials and methods used in Example 15.

Particle characterization: Size: IO-RNA-NPs were diluted 2000 times with cold PBS and measured with a NanoSight NS300 (Malvern). Particle size was calculated from over 1400 frames using 5 acquisitions per sample and 60 s per acquisition. Data was processed using NTA 3.3 Dev Build 3.3.104 (Camera Type: sCMOS). Selected plots and data are representative of four independent batches for each particle construct. Charge: Zeta potential was evaluated with a Nicomp ZLS Z3000. Reported measurements are averages of 5 cycles for each particle that are representative of 3 independent batches. Magnetism: Measurements of magnetism were made using a Quantum Design MPMS-3 Superconducting Quantum Interference Device (SQUID) magnetometer. Particles were prepared and analyzed in PBS at 2.5 mg/mL. Magnetization curves were obtained by applying a 10 Oe (0.8 kA/m) field at varying temperatures from 4K to 345K. RNA Binding: Liposomes were loaded with RNA at Liposome:RNA ratios of 15:1, 10:1, 5:1, 1:1, or 0:1 and incubated for 15 minutes to allow liposome formation before staining with RNA-loading buffer. 20 uL of IO-RNA-NPs were then loaded into each well of a 1% agarose gel and electrophoresed at 80V for 20 minutes. Free RNA was assessed with a ChemiDoc imaging system (Bio-Rad) and Image Lab software (Bio-Rad). The relative RNA binding capacity for each particle was calculated as: Bound RNA (%)*Total RNA (ug), where “Bound RNA” is calculated as 1−(Sample_{Band intensity}/RNA Alone_{Band intensity}). Cryogenic Electron Microscopy: Sample preparation for cryogenic transmission electron microscopy (Cryo-TEM) was performed in the Electron Microscopy Core of the University of Florida's Interdisciplinary Center for Biotechnology Research. Three microliter aliquots of suspended liposomes were applied to C-flat holey carbon grids (Protochips, Inc.) and vitrified using a Vitrobot™ Mark IV (FEI Co.) operated at 4° C. with ˜90% humidity in the control chamber. The vitrified sample was stored under liquid nitrogen and transferred into a Gatan cryo-holder (Model 626/70) for imaging. The sample was examined using a 4k×4k CCD camera (Gatan, Inc.) on a Tecnai (FEI Co.) G2 F20-TWIN Transmission Electron Microscope operated at a voltage of 200 kV using low dose conditions (˜20 e/Å2).

RNA Preparation and Labeling Green Fluorescent Protein (GFP) and OVA RNA were generated as previously described³¹. Isolated RNA was labeled with Cy3 and Cy5 dye using commercially available Arcturus Turbo Labeling kits (ThermoFisher Scientific) according to manufacturer instructions.

Cell culture DC2.4s are an immortalized dendritic cell line that were a kind gift from John Sampson, Duke University³⁶. B16F10-OVA is a murine melanoma cell line expressing the chicken ovalbumin gene (OVA) that was received as a kind gift from Dr. Richard G. Vile, PhD, at Mayo Clinic. Both cell types were cultured at 37° C. with 5% CO₂ in high glucose DMEM with pyruvate supplemented with 10% heat inactivated fetal bovine serum (FBS) and 1% Penicillin/Streptomycin (LifeTechnologies).

Synthesis of IO-RNA-NPs Liposome synthesis: Liposomes were prepared as previously described³¹. Briefly, the cationic liposome DOTAP was acquired from Avanti, Polar Lipids Inc. (Alabaster, Ala., USA) in the dehydrated form. 25-100 mg of the dehydrated liposome was mixed with Cholesterol 700000P (Avanti Polar Lipids) at a ratio of 3:1 DOTAP:Cholesterol in chloroform. The chloroform was evaporated in a nitrogen gas chamber, resulting in a thin layer of lipid. This lipid was then rehydrated to 2.5 mg/mL with PBS. For IO-RNA-NPs, the lipid cake was instead rehydrated with a dense solution of carboxylated IONPs (NanoMag) at a high concentration of 10 mg/mL before being brought to 2.5 mg lipid/mL with DPBS. The re-hydrated liposomes were then placed in a 50° C. water bath and vortexed every 10 minutes for 1 hour. Liposomes were then stored at room temperature overnight before being vortexed, placed in a bath sonicator for 5 minutes, and filtered through a 0.45 μm syringe filter (Whatman Puradisc) and afterwards a 0.20 μm syringe filter (PALL Acrodisc syringe filter with Supor membrane). IO-RNA-NP complex formation: IO-RNA-NPs were prepared as described previously for RNA-NPs³¹. 10 μg mRNA were added to 150 μg IO-RNA-NPs (per 2 million cells) in PBS buffer. The mixture was incubated at room temperature for 15 minutes to ensure complex formation before addition to DCs.

IO-RNA-NP transfection of DCs Overnight Transfection: IO-RNA-NPs were added to DCs in culture at 160 ug IO-RNA-NPs:2 million DCs overnight. 30 Minute Transfection: DCs were pulled to the bottom of 24-well plates by centrifugation for 1 minute at 100 rcf before addition of IO-RNA-NPs. Particles were left in media for 30 minutes in the presence or absence of a magnetic field created by neodymium iron boron (Nd₂Fe₁₄B) permanent magnetic disks. After 30 minutes, plates were again centrifuged at 100 rcf for 1 minute before IO-RNA-NPs were removed and replaced with fresh media.

Mice C57Bl/6, OT1 Transgenic (C57Bl/6-Tg(TcraTcrb)1100Mjb/J) and DsRed (B6.Cg-Tg(CAG-DsRed*MST)1Nagy/J) mice were purchased from Jackson Laboratories Animal procedures were approved by the University of Florida Institutional Animal Care and Use Committee (UFIACUC201607966).

Dendritic cell generation DCs were isolated from murine bone marrow based on previously established methods⁵⁰. Briefly, tibias and femurs were harvested from C57Bl/6 or DsRed mice and bone marrow was flushed using 25-gauge syringe with serum-containing media. Red blood cells were lysed with 10 mL Pharmlyse (BD Bioscience) before suspending mononuclear cells in complete DC media (RPMI-1640, 5% FBS, 1 M HEPES [LifeTechnologies], 55 mM b-mercaptoethanol [LifeTechnologies], 100 mM Sodium pyruvate [LifeTechnologies], 10 mM nonessential amino acids [LifeTechnologies], 200 mM L-glutamine [LifeTechnologies], 10 mg GM-CSF [R&D Systems], 10 mg IL4 [R&D Systems], 1% Penicillin/Streptomycin [LifeTechnologies]). Cells were then cultured in six-well plates at a concentration of 8×10⁵ cells/mL in a total volume of 3 mL/well. Non-adherent cells were discarded, and media was replaced at day 3. At day 7, non-adherent cells were collected and re-plated into 100 mm culture dishes at a density of 10⁶ cells/mL in a total volume of 5 mL/dish. Twenty-four hours later, non-adherent cells were collected, transfected with mRNA via electroporation, RNA Alone, RNA-NPs, or IO-RNA-NPs, and left overnight.

T Cell Generation Naïve OVA-specific T cells: T cells specific for OVA peptide epitopes 257-264 were generated from spleens of OT1 transgenic mice (C57Bl/6-Tg(TcraTcrb)1100Mjb/J). OT1 splenocytes were isolated by RBC lysis and suspended in PBS for immediate use in co-culture or treatment. Antigen-experienced T Cells: Antigen-experienced T cells were prepared as previously described⁴. Briefly, C57B16 mice received intradermal vaccination with OVA-pulsed DCs. Splenocytes were isolated from these mice 1 week after vaccination and cultured for 5 days with OVA-pulsed BMDCs in T cell media with IL-2 at a Splenocyte:DC ratio of 4,000,000:400,000. Activated T cells were split into new wells as they reached confluence.

Co-culture assays DCs were transfected with OVA or GFP mRNA via electroporation, co-culture (RNA-Alone), RNA-NPs, or IO-RNA-NPs. After 24 hours, treated DCs were co-cultured with naïve OT1 splenocytes or antigen-experienced OVA T cells in a 96 well plate at a T cell:DC ratio of 400,000:40,000. Supernatants were collected after 48 hours and evaluated with ELISA for interferon-γ (ebioscience).

Flow cytometric analysis Flow cytometry was performed using the BD Biosciences FACS Canto-II using antibodies from BD Biosciences, Biolegend, and Invitrogen. For in vitro experiments, DCs were harvested, washed with PBS, and stained for 20 minutes. Samples were then washed twice with PBS and suspended in FACS buffer. Cell counts and viability were assessed with Vi-Cell XR Cell Viability Analyzer (Beckman Coulter). For in vivo experiments, lymph nodes were harvested into cold PBS, diced with razor blade and digested in papain for 20 minutes at 37 C before filtering through a 70 μm cell strainer, washing with PBS, and staining for 20 minutes with appropriate antibodies. Counting beads were added to each tube immediately before flow analysis.

MRI imaging and analysis Image Acquisition: MRI imaging was performed on a 11T MRI magnet (Magnex Scientific, 11.1 T/40 cm bore) equipped with a Bruker AV3 HD console and Paravision 6.01 software using a custom built 30 mm ID quadrature birdcage transmit-receiver volume coil at the UF AMRIS facility 18-72 hours after intradermal injection of IO-RNA-NP-loaded DCs in the inguinal area. Mice were imaged under isoflurane anesthesia and monitored via continuous measurements of body temperature and respirations according to UFIACUC201607966. Circulating warm water from a temperature-controlled water heater was used to maintain body temperature. Two-dimensional MRI sequences with an image size of 192×192, field of view of 25.08 mm×25.921 mm, section thickness of 1.0 mm and 6 slices were collected with respiratory triggering for a variety of image parameters. T2*-weighted images were collected using the following parameters to evaluate IONP-dependent changes in hypointensity in lymph nodes: repetition time (TR)=90 ms, echo time (TE)=5 ms, flip angle=10° with fat saturation. A set of five other imaging sequences was taken to evaluate lymph node size. Sequence 1 (T2*): TR=90 ms, TE=3 ms, flip angle=10°. Sequence 2 (T2* with fat sat): TR=90 ms, TE=3 ms, flip angle=10° with fat saturation. Sequence 3 (T2_407/17): TR=407.204 ms, TE=17 ms, echo spacing=4.25, RARE factor=4, with fat saturation. Sequence 4 (T2 with fat sat): TR=500 ms, TE=14 ms, echo spacing=7 ms, RARE factor=4. Sequence 5 (T2): TR=3000 ms, TE=28 ms, echo spacing=3 ms, RARE factor=8, with fat saturation.

Image analysis: Image analysis was completed using ImageJ (NIH). Researchers blinded to treatment group manually created regions of interest around each lymph node for each slice in which it appeared. MRI intensity in each lymph node was calculated as the average of lymph node intensities across all slices in which the lymph nodes appeared for T2*-weighted MRI sequences. Lymph node volume was calculated as the product of the slice thickness (1 mm) and the summed areas of each lymph node for each slice (e.g. V=Slice Thickness*(Area_{Slice 1}+Area_{Slice 2}+Area_{Slice 3} Area_{Slice m})). Relative size was calculated for each imaging sequence as the volume of the treated lymph node divided by the volume of the untreated lymph node on the contralateral side. Relative volume was calculated for all 6 imaging sequences and reported as the average relative volume across imaging sequences. Relative intensity was calculated as the MRI intensity of the treated lymph node divided by the MRI intensity of the untreated lymph node for T2*-weighted MRI images.

Treatment models Tumor implantations: B16F10-OVA cells were harvested with 0.05% trypsin (Gibco), washed once in serum-containing medium, and washed once in Dulbecco's phosphate-buffered saline (DPBS). Cell pellets were resuspended in DPBS at a concentration of 10⁷ cells/mL. 1 million B16F10-OVA cells were subcutaneously injected with a 25-gauge syringe into the left flank of C57Bl/6 mice anesthetized with isoflurane. Subcutaneous tumors were measured every 2-4 days with WESTWARD Digital Caliper Animals bearing subcutaneous tumors that reached humane endpoint were euthanized. Adoptive Cellular Therapy: Naïve or antigen experienced T cells were generated as described above, suspended in PBS at 100 million/mL, and injected into tumor-bearing mice at 100 uL per mouse. DC Vaccines: RNA-pulsed DCs prepared as described above were collected and suspended in PBS at a final concentration of 1×10⁷ cells/mL. 50 uL was administered intradermally in the inguinal area for each treated mouse.

Immunofluorescence DC2.4s were imaged in PBS 24 hours after incubation with Cy3-labelled mRNA. Images were taken with an Olympus IX70 Inverted Fluorescent Microscope.

Gene expression analysis BMDCs were harvested from three independent samples per treatment group 24 hours after transfection with GFP mRNA via electroporation, RNA-NPs, or IO-RNA-NPs at 10 μg mRNA per 2 million cells. RNA was then isolated from each sample using commercially available RNeasy mini kits (Quiagen, cat #74104) as per the manufacturer instructions and analyzed for purity using a NanoDrop 2000 spectrophotometer (ThermoFisher Scientific) and Agilent 2100 BioAnalyzer. RNA was prepared for directional sequencing using Illumina RNA-Sequencing libraries (Poly A) at the UF Interdisciplinary Center for Biotechnology Research (ICBR). Paired end RNA sequencing with 100 cycles in 2 lanes were completed with an Illumina HiSeq 3000. Gene set enrichment was evaluated using the gene set enrichment analysis (GSEA) software available from the Broad Institute (genepattern.broadinstitute.org). Nine gene sets were selected from the C7 Immunologic Signatures gene set collection based on the hypothesis that RNA uptake in endosomes initiates a toll-like receptor-dependent DC activation phenotype.

Statistical analysis Data are summarized as the mean±standard deviation for in vitro experiments and mean±SEM for tumor-growth experiments. Unpaired data is analyzed with two-tailed unpaired student's t tests or ANOVA with Tukey's tests for multiple comparisons. Paired data is analyzed with Wilcoxon matched-pairs rank sum test for experiments with n>3 and two-tailed paired student's t tests when n<3. Tumor growth over time is measured with two-way ANOVA. Survival is measured with a log-rank test. Statistical analysis was conducted using GraphPad Prism version 6. Statistical significance was defined as p<0.05. Linear correlations were evaluated with Pearson's correlation coefficient.

References Cited The following references are cited throughout Examples 15 and 16: (1) Rosenberg, S. A., et al., Clin Cancer Res 17, 4550-4557 (2011); (2) Schadendorf, D., et al., J Clin Oncol 33, 1889-1894 (2015); (3) van der Burg, et al., Rev Cancer 16, 219-233 (2016); (4) Garon et al., N Engl J Med 372, 2018-2028 (2015); (5) Larkin, J., et al., N Engl J Med 373, 23-34 (2015); (6) Hellmann, M. D., et al., Lancet Oncol 18, 31-41 (2017); (7) Grippin et al., Translational Nanoparticle Engineering for Cancer Vaccines. Oncolmmunology, 00-00 (2017). (8) Nishino et al., Nat Rev Clin Oncol 14, 655-668 (2017); (9) Lesterhuis, W. J., et al., Nat Rev Drug Discov 16, 264-272 (2017); (10) Mitchell, D. A., et al., Nature 519, 366-369 (2015); (11) Srinivas, M., et al., Adv. Drug Deliv. Rev. 62, 1080-1093 (2010); (12) de Vries, I. J., et al., Nat Biotechnol 23, 1407-1413 (2005); (13) Verdijk, P., et al., Int J Cancer 120, 978-984 (2007); (14) Noh et al., Biomaterials 32, 6254-6263 (2011); (15) Cho et al., Nat Nanotechnol 6, 675-682 (2011); (16) Mou et al., Int J Nanomedicine 6, 2633-2640 (2011); (17) de Chickera, S., et al., Int Immunol 24, 29-41 (2012); (18) Zhang, Z., et al., Radiology 274, 192-200 (2015); (19) Batich, K. A., et al., Clin Cancer Res 23, 1898-1909 (2017); (20) Anguille, S., et al., Blood 130, 1713-1721 (2017); (21) Wilgenhof, S., et al., J Clin Oncol 34, 1330-1338 (2016); (22) Bol, K. F., et al., Oncoimmunology 4, e1019197 (2015); (23) Aarntzen, E. H., et al., Clin Cancer Res 18, 5460-5470 (2012); (24) Phua, K. K. L., et al., Nanoscale 6, 7715-7729 (2014); (25) Anselmo, A. C. & Mitragotri, S. Nanoparticles in the clinic. Bioeng Transl Med 1, 10-29 (2016); (26) Shi, J., et al., Nat Rev Cancer 17, 20-37 (2017); (27) de Vries, I. J. M., et al, Nat Biotech 23, 1407-1413 (2005); (28) Ahrens, E. T. & Bulte, J. W. M. Nat Rev Immunol 13, 755-763 (2013); (29) Bulte, J. W. M. American Journal of Roentgenology 193, 314-325 (2009); (30) Kranz, L. M., et al., Nature 534, 396-401 (2016); (31) Sayour, E. J., et al. Oncolmmunology, 00-00 (2016); (32) Sayour, E. J., et al. Nano Lett (2018); (33) Kauffman, K. J., et al. Nano Lett 15, 7300-7306 (2015); (34) Sayour, E. J., et al. Oncoimmunology 6, e1256527 (2017); (35) Soema, P. C., Willems, G. J., Jiskoot, W., Amorij, J. P. & Kersten, G. F. Eur J Pharm Biopharm 94, 427-435 (2015); (36) Shen, Z., Reznikoff, G., Dranoff, G. & Rock, K. L. J Immunol 158, 2723-2730 (1997). (37) Scherer, F., et al. Gene Ther 9, 102-109 (2002). (38) Mah, C., et al. Mol Ther 6, 106-112 (2002). (39) Gilboa, E. & Vieweg, J. Immunol Rev 199, 251-263 (2004); (40) Van Tendeloo, V. F., et al. Blood 98, 49-56 (2001); (41) Blasius, A. L. & Beutler, B. Immunity 32, 305-315 (2010); (42) Crozat, K. & Beutler, B. Proc Natl Acad Sci USA 101, 6835-6836 (2004); (43) de Lima, M. C., et al. Mol. Membr. Biol. 16, 103-109 (1999); (44) Lim, Y. T., et al. Small 4, 1640-1645 (2008); (45) Jin, H., et al. Theranostics 6, 2000-2014 (2016); (46) Mackay, P. S., et al. Nanomedicine 7, 489-496 (2011); (47) Wang, Z., et al. Immunity 49, 80-92 e87 (2018); (48) Zanganeh, S., et al. Nat Nanotechnol 11, 986-994 (2016); (49) Ragelle, H., Danhier, F., Preat, V., Langer, R. & Anderson, D. G. Expert Opin Drug Deliv 14, 851-864 (2017); (50) Flores, C., et al. Oncolmmunology 4, e994374 (2015).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed:

1. A liposome comprising ribonucleic acid (RNA) molecules, a lipid mixture comprising DOTAP and cholesterol, and iron oxide nanoparticles (IONPs).

2. The liposome of claim 1, wherein each IONP in the core has a diameter of about 10 nm to about 200 nm.

3. The liposome of claim 2, wherein each IONP has a diameter or about 60 nm to about 140 nm.

4. The liposome of any one of claims 1 to 3, wherein the mass of the IONPs is about 1% to about 30% of the total liposome mass.

5. The liposome of claim 4, wherein the mass of the IONPs is about 5% to about 25% of the total liposome mass, optionally, about 10% to about 15% of the total liposome mass.

6. The liposome of claim 5, wherein the mass of the IONPs is about 12%±3% of the total liposome mass.

7. The liposome of any one of the preceding claims, wherein the DOTAP and cholesterol are present in the lipid mixture at a DOTAP:cholesterol ratio of about 3:1 by mass.

8. The liposome of any one of the preceding claims, wherein the IONPs are present in the core of the liposome.

9. The liposome of any one of the preceding claims, wherein the IONPs are dispersed throughout the liposome.

10. A liposome comprising ribonucleic acid (RNA) molecules and a lipid mixture comprising DOTAP and cholesterol, wherein the DOTAP and cholesterol are present in the lipid mixture at a DOTAP:cholesterol ratio of about 3:1 by mass.

11. The liposome of any one of the preceding claims, having a diameter between about 80 nm to about 500 nm, optionally, a diameter between about 90 nm to about 300 nm.

12. The liposome of any one of the preceding claims, having an overall surface net charge of about 20 mV to about 50 mV, optionally, an overall surface net charge of about 40 mV to about 50 mV.

13. The liposome of any one of the preceding claims, wherein the cholesterol is more than 12% and less than 37% of the total lipid mass of the lipid mixture.

14. The liposome of claim 13, wherein the cholesterol is about 15% to about 35% of the total lipid mass of the lipid mixture, optionally, about 20% to about 30% of the total lipid mass of the lipid mixture.

15. The liposome of claim 14, wherein the cholesterol is about 25%±3% of the total lipid mass of the lipid mixture.

16. The liposome of any one of the preceding claims, wherein the DOTAP is at least 50% of the total lipid mass of the lipid mixture, optionally, about 63% to about 88% of the total lipid mass of the lipid mixture.

17. The liposome of claim 16, wherein the DOTAP is about 75%±5% of the total lipid mass of the lipid mixture.

18. The liposome of any one of the preceding claims, wherein, when the lipid mixture comprises a third lipid which is different from DOTAP and cholesterol, the third lipid is less than about 10% or less than about 5% of the total lipid mass of the lipid mixture.

19. The liposome of any one of the preceding claims, wherein the lipid mixture consists essentially of DOTAP and cholesterol.

20. The liposome of any one of the preceding claims, comprising less than or about 10 μg RNA molecules per 150 μg liposome.

21. The liposome of any one of the preceding claims, wherein the RNA molecule encodes a protein or is an antisense molecule.

22. The liposome of claim 21, wherein the protein is selected from the group consisting of: a tumor antigen, a cytokine, or a co-stimulatory molecule.

23. The liposome of claim 21, wherein the RNA molecule is an antisense molecule and the antisense molecule is an siRNA, shRNA, miRNA, or any combination thereof.

24. The liposome of any one of the previous claims, comprising a mixture of RNA molecules.

25. The liposome of claim 24, wherein the mixture of RNA molecules is RNA isolated from cells from a human.

26. The liposome of claim 25, wherein the human has a tumor and the mixture of RNA is RNA isolated from the tumor of the human, optionally, wherein the tumor is a malignant brain tumor, optionally, a glioblastoma, medulloblastoma, diffuse intrinsic pontine glioma, or a peripheral tumor with metastatic infiltration into the central nervous system.

27. The liposome of any one of claims 10-26, further comprising IONPs.

28. The liposome of claim 27, wherein each IONP in the core has a diameter of about 10 nm to about 200 nm, optionally, about 60 nm to about 140 nm.

29. The liposome of claim 27 or 28, wherein the mass of the IONPs is about 1% to about 30% of the total liposome mass, optionally, about 5% to about 25% of the total liposome mass, optionally, about 10% to about 15% of the total liposome mass.

30. The liposome of claim 29, wherein the mass of the IONPs is about 12%±3% of the total liposome mass.

31. The liposome of any one of claims 27 to 30, wherein the IONPs are present in the core of the liposome.

32. The liposome of any one of claims 27 to 30, wherein the IONPs are dispersed throughout the liposome.

33. A method of making a liposome, comprising (A) mixing DOTAP and cholesterol at a DOTAP:cholesterol ratio of about 3:1 by mass to form a lipid mixture, (B) drying the lipid mixture, (C) rehydrating the lipid mixture with a rehydration solution to form a rehydrated lipid mixture, (D) incubating the rehydrated lipid mixture at a temperature greater than about 40° C. and intermittently vortexing the rehydrated lipid mixture to form liposomes.

34. The method of claim 33, further comprising incubating the liposomes for more than 12 hours after step (D), optionally, further comprising incubating the liposomes for more than 12 hours at about 20° C. to about 30° C. or at about 2° C. to about 6° C.

35. The method of claim 33 or 34, further comprising (i) sonicating the liposomes and/or filtering the liposomes through a filter of at least 150 nm, optionally, wherein the liposomes are filtered through a 200 nm filter and/or a 450 nm filter, (ii) incubating the liposomes with RNA molecules

36. The method of claim 35, wherein the liposomes are filtered through a 450 nm filter and a 200 nm filter, optionally, wherein the liposomes are sequentially filtered through a 450 nm filter followed by 200 nm filter.

37. The method of any one of claims 33 to 36, wherein (i) about 7.5 mg±0.75 mg DOTAP and about 2.5 mg±0.25 mg cholesterol are mixed to form the lipid mixture, (ii) DOTAP and cholesterol are dissolved in chloroform to form the lipid mixture, (iii) nitrogen gas is used to dry the lipid mixture, (iv) the rehydration solution is a buffer, optionally, a phosphate buffered saline (PBS), (v) the rehydrated lipid mixture is incubated in a water bath at a temperature of about 50° C. and vortexed about every 10 minutes to form liposomes, (vi) or a combination thereof.

38. The method of any one of claims 33 to 37, wherein the lipid mixture or the rehydration solution further comprises iron oxide nanoparticles (IONPs) or the method further comprises adding IONPs the lipid mixture or the rehydration solution, optionally, wherein each IONP has a diameter of about 10 nm to about 200 nm, optionally, about 60 nm to about 140 nm or about 10 nm to about 30 nm.

39. The method of claim 38, wherein the lipid mixture or rehydration solution comprises at least about 1 μg IONPs per 10 mg lipid mixture, at least about 100 μg IONPs per 10 mg lipid mixture, at least about 1 mg IONPs per 10 mg lipid mixture, or at least about 1.5 mg IONPs per 10 mg lipid mixture, optionally, wherein the lipid mixture or rehydration solution comprises no more than about 5 mg IONPs per 10 mg lipid mixture.

40. The method of any one of claims 33 to 39, comprising incubating the liposomes with RNA molecules, optionally, wherein (i) about 5 μg RNA molecules is incubated with about every 75 μg lipids of the liposomes, (ii) the method comprises incubating the liposomes with RNA molecules at a RNA molecule:DOTAP ratio of about 1:15 by mass, (iii) wherein about 10 μg RNA molecules is incubated with about every 150 μg liposomes when the liposomes comprise IONPs, or (iv) a combination thereof.

41. A liposome made by the method of any one of claims 33 to 40.

42. A cell comprising a liposome of any one of claims 1 to 32 and 41.

43. The cell of claim 42, which is an antigen presenting cell (APC), optionally, a dendritic cell (DC).

44. A population of cells, wherein at least 50% of the population are cells according to any one of claim 42 or 43.

45. A composition comprising a liposome of any one of claims 1 to 32 and 41, a cell of claim 42 or 43, a population of cells of claim 44, or any combination thereof, and a pharmaceutically acceptable carrier, excipient or diluent.

46. The composition of claim 45, comprising a plurality of liposomes, wherein at least 50% of the liposomes have a diameter between about 100 nm to about 250 nm.

47. A method of delivering RNA molecules to cells, comprising incubating the cells with the liposomes of any one of claims 1 to 32 and 41.

48. The method of claim 47, wherein the cells are antigen-presenting cells (APCs), optionally, dendritic cells (DCs).

49. The method of claim 47 or 48, wherein the liposomes comprise IONPs.

50. The method of claim 49, wherein the cells are incubated with the liposomes in the presence of a magnetic field, optionally, a static magnetic field or an oscillating magnetic field.

51. The method of claim 50, wherein the cells are incubated with the liposomes in the presence of a magnetic field for time of less than about 2 hours or less than about 1 hour, optionally, wherein the cells are incubated with the liposomes in the presence of a magnetic field for about 30 minutes±10 minutes.

52. A method of treating a subject with a disease, comprising delivering RNA molecules to cells of the subject by a method of any one of claims 47 to 51.

53. The method of claim 52, wherein RNA molecules are ex vivo delivered to the cells and the cells are administered to the subject.

54. A method of treating a subject with a disease, comprising administering to the subject a composition of claim 45 or 46 in an amount effective to treat the disease in the subject.

55. The method of claim 54, wherein the disease is cancer, optionally, wherein the cancer is located across the blood brain barrier.

56. The method of claim 54 or 55, wherein the subject has a tumor located in the brain.

57. The method of claim 57, wherein the tumor is a low grade glioma or a high grade glioma, e.g., a grade III astrocytoma or a glioblastoma, a medulloblastoma or a diffuse intrinsic pontine glioma.

58. The method of any one of claims 54 to 76, wherein the composition comprises liposomes.

59. The method of claim 58, wherein the composition is intravenously administered to the subject.

60. The method of any one of claims 54 to 59, wherein the composition comprises cells comprising the liposome.

61. The method of claim 60, wherein the composition comprising the cells comprising the liposome is intradermally administered to the subject, optionally, wherein the composition is intradermally administered to the groin of the subject.

62. The method of claim 60 or 61, wherein the cells are APCs, optionally, dendritic cells (DCs).

63. The method of claim 62, wherein the DCs are isolated from WBCs obtained from the subject.

64. The method of any one of claims 47 to 63, wherein the RNA molecules of the liposomes encode a tumor antigen and/or are isolated from tumor cells, optionally, wherein the tumor cells are cells of a tumor of the subject.

65. The method of any one of claims 47 to 86, wherein the liposomes comprise IONPs and the method further comprises tracking migration of the cells comprising the liposomes within the subject.

66. The method of claim 65, wherein the tracking comprises magnetic resonance imaging (MRI), optionally, wherein the tracking comprises conducting MRI on one or more lymph nodes of the subject, optionally, the inguinal lymph nodes, wherein, optionally, MRI is conducted on the lymph nodes before and after administration of the composition or the cells.

67. The method of claim 66, comprising comparing the T2*-weighted MRI intensity of the lymph node comprising DCs transfected with liposomes comprising IONPs to the T2*-weighted MRI intensity of a control, untreated lymph node.

68. The method of claim 66 or 67, comprising measuring lymph node size of the subject via MRI, optionally, comprising comparing the lymph node size of the lymph node comprising DCs transfected with liposomes comprising IONPs lymph node compared to the lymph node size of the a control, untreated lymph node.

69. A method of tracking dendritic cell (DC) migration to a lymph node in a subject, comprising (i) treating the subject in accordance with the method of any one of claims 52 to 68, wherein the cells are DCs and the liposomes comprise IONPs, and (ii) performing magnetic resonance imaging (MRI) on one or more lymph nodes of the subject.

70. The method of claim 69, comprising determining the T2*-weighted MRI intensity of one or more lymph nodes, wherein lymph nodes exhibiting a reduction in T2*-weighted MRI intensity, relative to the T2*-weighted MRI intensity of a control, untreated lymph node, represent lymph nodes to which DCs migrated.

71. The method of claim 69 or 70, wherein one or more lymph nodes are the inguinal lymph nodes of the subject, optionally, wherein the composition is intradermally administered to the groin of the subject.

72. The method of any one of claims 69 to 71, wherein MRI is conducted on the lymph nodes before and after administration of the composition or the cells, optionally, wherein MRI is conducted before and about 48 hours after administration and, optionally, about 72 hours after administration.

73. The method of any one of claims 69 to 72, comprising comparing the T2*-weighted MRI intensity of the lymph node comprising DCs transfected with liposomes comprising IONPs to the T2*-weighted MRI intensity of a control, untreated lymph node.

74. The method of any one of claims 69 to 73, comprising measuring lymph node size of the subject via MRI, optionally, comprising comparing the lymph node size of the lymph node comprising DCs transfected with liposomes comprising IONPs to the lymph node size of the a control, untreated lymph node.

75. A method of determining a subject's therapeutic response to dendritic cell (DC) vaccination therapy in a subject, comprising (i) treating the subject in accordance with the method of any one of claims 52 to 68, wherein the cells are DCs and the liposomes comprise IONPs, and (ii) tracking DC migration to a lymph node in accordance with any one of claims 69 to 74, wherein, when T2*-weighted MRI intensity of treated lymph nodes is reduced, the DC vaccination therapy is determined to lead to a positive therapeutic response in the subject.

76. The method of claim 75, wherein the positive therapeutic response comprises prolonged progression free and overall survival of the subject for at least 4 weeks post-administration of therapy.

77. The method of claim 76, wherein the positive therapeutic response comprises prolonged progression free and overall survival of the subject for at least 8 to 12 weeks post-administration of therapy.

78. A method of monitoring therapeutic response to dendritic cell (DC) vaccination therapy in a subject, comprising tracking DC migration to a lymph node in accordance with any one of claims 69 to 74 at a first time point and at a second time point, wherein, when T2*-weighted MRI intensity of treated lymph nodes is reduced at the second time point relative to the T2*-weighted MRI intensity of the treated lymph nodes at the first time point, the therapeutic response to DC vaccination therapy is effective.

79. A method of delivering RNA to cells in a microenvironment of a tumor, optionally a brain tumor, comprising intravenously administering a composition of claim 45 or 46, wherein the composition comprises the liposome.

80. The method of claim 79, wherein the liposome comprises siRNA targeting a protein of a immune checkpoint pathway, optionally, PDL1.

81. The method of claim 79 or 80, wherein the cells in the microenvironment are antigen-presenting cells (APCs), optionally, tumor associated macrophages.

82. A method of activating antigen-presenting cells in a brain tumor microenvironment, comprising intravenously administering a composition of claim 45 or 46, wherein the composition comprises the liposome.

83. A method of increasing dendritic cell (DC) migration to a lymph node in a subject, comprising administering to the subject a composition of claim 45 or 46, in an amount effective to increase DC migration to the lymph node.

84. A method of enhancing in a subject an immune response against a tumor or cancer, comprising administering to the subject a composition of claim 45 or 46 in an amount effective to enhance the immune response in the subject.
